(19) United States
(12) Patent Application Publication
(10) Pub. No.: US 2023/0404933 A1
(43) Pub. Date: Dec. 21, 2023

(54) NON-CATIONIC SOFT POLYPHENOL NANOCAPSULES FOR EFFECTIVE SYSTEMIC DELIVERY OF SMALL INTERFERING RNA (siRNA) FOR CANCER TREATMENT

(71) Applicant: Purdue Research Foundation, West Lafayette, IN (US)

(72) Inventors: Yoon Yeo, West Lafayette, IN (US); Hyungjun Kim, Gyeongbuk (KR)

(73) Assignee: Purdue Research Foundation, West Lafayette, IN (US)

(21) Appl. No.: 18/034,969

(22) PCT Filed: Oct. 27, 2021
(86) PCT No.: PCT/US2021/056730

§ 371 Date: May 2, 2023

Related U.S. Application Data
(60) Provisional application No. 63/110,387, filed on Nov. 6, 2020.

(51) Int. Cl. A61K 9/51; A61K 45/00; A61K 47/69; A61K 48/00

(52) U.S. Cl. CPC A61K 9/5146; A61K 48/0041; A61K 47/6935; A61K 45/00

(57) ABSTRACT

The present disclosure generally relates to a composition matter and a method for cancer treatment. In particular, a composition of soft, non-cationic nanocapsules, termed Nanosac, for systemic delivery of siRNA. Nanosac is produced by sequential attachment of siRNA and polydopamine on a sacrificial MSN core, followed by removal of the MSN. Encapsulating siRNA in the capsules, Nanosac avoids the issues common to cationic gene carriers, such as toxicity and non-specific protein binding while protecting siRNA from RNase. Nanosac entered tumor cells by caveolae-mediated endocytosis, likely via albumin recruited from serum, trafficked to the cytosol, and silenced target genes.

### CROSS REFERENCE TO RELATED APPLICATIONS

This present patent application relates to and claims the priority benefit of U.S. Provisional Application Ser. No. 63/110,387, filed Nov. 6, 2020, the content of which is hereby incorporated into this disclosure by reference in its entirety.

### GOVERNMENT SUPPORT CLAUSE

This invention was made with government support under CA199663 awarded by the National Institutes of Health. The government has certain rights in the invention.

### STATEMENT OF SEQUENCE LISTING

A computer-readable form (CRF) of the Sequence Listing is submitted concurrently with this application. The file, entitled 68868-02_Seq_Listing_ST25_txt, is generated on Oct. 14, 2021. Applicant states that the content of the computer-readable form is the same and the information recorded in computer readable form is identical to the written sequence listing.

### TECHNICAL FIELD

The present disclosure generally relates to a composition matter and a method for cancer treatment. In particular, a composition matter of soft, non-cationic nanocapsules, termed Nanosac, for systemic delivery of a biologic or a small molecule drug compound.

### BACKGROUND AND SUMMARY

This section introduces aspects that may help facilitate a better understanding of the disclosure. Accordingly, these statements are to be read in this light and are not to be understood as admissions about what is or is not prior art.

Small interfering RNA (siRNA) is a short double-stranded RNA, 20-25 base pairs in length, which downregulates specific gene expression by inducing mRNA degradation. Due to the high efficiency and specificity, siRNA has been actively pursued as a therapeutic agent for cancer, viral infections, and autoimmune diseases (1-4). However, the challenges in developing effective siRNA therapeutics are their instability in circulation and inability to enter cells (5-7). For in vivo delivery, siRNA is covalently modified or encapsulated in nanoscale carriers, such as cationic liposomes, polymeric nanocarriers, and inorganic particles (8-10). Recently, two siRNA products received the approval of the U.S. Food and Drug Administration: patisiran (ONPATTRO™), siRNA encapsulated in a lipid nanoparticle (NP), for the treatment of hereditary transthyretin-mediated amyloidosis (11), and givosiran (Givlaari™), siRNA covalently linked to a ligand targeting hepatocytes, for the treatment of acute hepatic porphyria (12). These new developments demonstrate that therapeutic delivery of siRNA is possible when accompanied by carriers that can protect siRNA and facilitate its cellular uptake. However, most existing carriers have shown limited success in systemic delivery of siRNA to the organs other than the liver or lungs, the filtering organs with the reticuloendothelial system (RES) (13, 14). Without a reliable carrier for systemic delivery, siRNA will remain sidelined in the therapy of undruggable diseases, which would have significantly benefited from its efficiency and specificity otherwise. There are unmet needs in efficient and effective delivery of siRNA for effective treatment of ever developing and changing cancers.

### BRIEF DESCRIPTION OF THE DRAWINGS

The above and other objects, features, and advantages of the present invention will become more apparent when taken in conjunction with the following description and drawings wherein identical reference numerals have been used, where possible, to designate identical features that are common to the figures, and wherein:

FIGS. 1A-1D. Preparation and characterization of Nanosac. (FIG. 1A) Schematic of O/siRNA/pD (Nanosac) preparation. MSN: mesoporous silica nanoparticles; MSN^{a}: MSN conjugated with (3-Aminopropyl)triethoxysilane (APTES); MSN^{a}/siRNA: MSN^{a} with siRNA loaded on the surface; MSN^{a}/siRNA/pD: MSN^{a}/siRNA covered with pD; O/siRNA/pD (Nanosac): siRNA-loaded nanocapsules after MSN core removal. (FIG. 1B) Transmission electron micrographs (TEM) of Nanosac and the precursors. Visualized by negative staining with 1% uranyl acetate. Scale bars: 50 nm. (FIG. 1C) Zeta potential and Z-average of Nanosac and the precursors (n=3 independently and identically prepared batches, mean±s.d) (FIG. 1D) Force-distance curves for MSN^{a}/pD and O/pD (Nanosac) measured by AFM, and Young's moduli of MSN^{a}/PD and Nanosac (n=15 tests of a representative batch, mean±s.d.). ****: p<0.0001 vs. MSN^{a}/pD by unpaired t-test.

FIGS. 2A-2F. Stability of Nanosac. (FIG. 2A) Gel electrophoresis of siLuc, MSN^{a}/siLuc, MSN^{a}/siLuc/pD, and Nanosac with/without RNase or SDS challenge; (FIG. 2B) Gene silencing by MSN^{a}/siLuc, MSN^{a}/siLuc/pD, and Nanosac with/without RNase challenge (n=3 test of a representative batch, mean±s.d.). ***: p<0.001; ns: not significant by Sidak's multiple comparisons test following one-way ANOVA. Gene silencing by MSN^{a}/siRNA, MSN^{a}/siRNA/pD, and Nanosac, measured after 48 h treatment in complete medium. (FIG. 2C) siGAPDH in CT26 cells, (FIG. 2D) siLuc in 4T1-Luc cells, and (FIG. 2E) siPD-L1 in IFN-γ-activated CT26 cells. (n=3 test of a representative batch, mean±s.d.). ***: p<0.001, ****: p<0.0001 vs. No siRNA by Dunnett's multiple comparisons test following two-way ANOVA. (f) Top: Representative western blot of PD-L1 expression in IFN-γ-activated CT26 cells by MSN^{a}/siPD-L1, MSN^{a}/siPD-L1/pD, and Nanosac. Bottom: Quantitative presentation of western blotting.

FIGS. 3A-3I. Endocytosis pathway of NPs. FIG. 3A: Effects of endocytosis inhibitors on NP uptake in serum-supplemented medium. CT26 cells were preincubated for 30 min with each inhibitor at a subtoxic concentration, followed by treatment of NPs in 10% FBS-supplemented medium for 2 h (n=3 tests of a representative batch, mean±s.d.). ****: p<0.0001, ns: not significant vs. 37° C. by Dunnett's multiple comparisons test following two-way ANOVA. CPZ: chloropromazine. Albuminylation of NPs. FIG. 3B: Zeta potential of MSN^{a}, MSN^{a}/pD, and Nanosac before and after incubation in 50% FBS (n=3 tests of a representative batch, mean±s.d.). FIG. 3C: SDS-PAGE of protein corona composition formed on MSN^{a}, MSN^{a}/pD, and Nanosac. NPs (4 mg/mL) were incubated in 50% FBS for 2 h and rinsed with PBS twice. FIG. 3D: Spectral counts of proteins, analyzed by LC-MS/MS, bound on the MSN^{a}, MSN^{a}/pD, and Nanosac after 2 h exposure to 50% FBS.

FIG. 3E: Representative SDS-PAGE gel of albumin after pulse proteolysis. Native albumin (nAlb), denatured albumin (dAlb), MSN^{a} incubated with albumin (MSN^{a}+Alb) and MSN^{a}/pD with albumin (MSN^{a}/pD+Alb) were treated with thermolysin for 3 min. Lane 1: nAlb; Lane 2: dAlb; Lane 3: MSN^{a}+Alb; Lane 4: MSN^{a}/pD+Alb. % digestion albumin was defined as (1-albumin band intensity after proteolysis/albumin band intensity before proteolysis)×100. n=3 independently and identically performed experiments (mean±s.d.). ***: p<0.001 and ****: p<0.0001 vs. nAlb by Dunnett's multiple comparisons test following one-way ANOVA. Intracellular trafficking of NPs. FIG. 3F: Confocal microscope images locating cy5-labeled MSN^{a}, MSN^{a}/pD, and Nanosac relative to lysosomes in CT26 cells. Green: Lysotracker (lysosome); Red: cy5-labeled NPs; Blue: Hoechst 33342 (nuclei). Scale bars: 10 μm. FIG. 3G: Pearson's correlation coefficients indicating the degree of NP/lysosome colocalization in confocal images: R=1 (perfect colocalization), R=0 (no colocalization). n=5 tests of a representative batch (mean±s.d). ***: p<0.001 and ****: p<0.0001 by Tukey's multiple comparisons test following one-way ANOVA. FIG. 3H: Fluorescence intensity profiles along the white lines in (f). FIG. 3I: Release kinetics of siRNA from Nanosac, performed in different pHs or H₂O₂ (100 PM) with constant agitation at 37° C. n=3 tests with representative batches (mean±s.d.).

FIGS. 4A-4E. Comparison of MSN^{a}-cy5/pD and Nanosac in macrophage uptake, extravasation, and tumor spheroid penetration. (FIG. 4A) Quantitative measurement (Top panel) and confocal microscope images (Bottom panel) of J774a.1 macrophage taking up MSN^{a}-cy5/pD and Nanosac. n=3 tests of a representative batch (mean±s.d). ***: p<0.001 and ****: p<0.0001 by Sidak's multiple comparisons test following two-way ANOVA. Green: Wheat Germ Agglutinin (cell membrane); Red: cy5-labeled NPs; Blue: Hoechst 33342 (nuclei). Scale bar: 50 μm. (FIG. 4B) Time-lapse intravital microscopic images of MSN^{a}-cy5/pD and Nanosac circulating in CT26 tumor-bearing BALB/c mice. Green: Dextran-FITC (locating blood vessel), Red: cy5-labeled NPs. (FIG. 4C) Z-section images of CT26 tumor spheroids incubated with MSN^{a}-cy5/pD or Nanosac. Scale bars: 500 μm. (FIG. 4D) Spheroid depth-wise fluorescence intensity profiles. n=3 tests of a representative batch (mean±s.d). ****: p<0.0001 vs. MSN^{a}-cy5/pD by Sidak's multiple comparisons test following two-way ANOVA. (FIG. 4E) Horizontal fluorescence intensity profile at 160 μm.

FIGS. 5A-5G. Anti-tumor activity of 5% dextrose (D5W), MSN^{a}/siPD-L1/pD, and Nanosac in Balb/c mice bearing CT26 tumors (siPD-L1: 0.75 mg/kg/time, q2dx10). (FIG. 5A) Average tumor size (mm³). *: p<0.05 and ****: p<0.0001 between average tumor sizes on day 20 post-first injection by Tukey's multiple comparisons test following two-way repeated measures ANOVA. (FIG. 5B) Average body weight (g). Arrowheads indicate times of treatment. n=5 mice per treatment (mean±s.d). (FIG. 5C) Expression of PD-L1 in CT26 tumors (FIG. 30). n=4 mice per group (mean±s.d). *: p<0.05; ns: not significant vs. D5W by Dunnett's multiple comparisons test following one-way ANOVA. (FIG. 5D) % CD8⁺ cells, % CD4⁺ cells, and CD8⁺/CD4⁺ ratio in TDLNs of treated animals. n=5 mice per treatment (mean±s.d). *: p<0.05, ***: p<0.001, ****: p<0.0001, and ns: not significant vs. D5W by Dunnett's multiple comparisons test following one-way ANOVA. (FIG. 5E) Fluorescence micrographs of tumor sections showing FITC-lectin-stained vessels (green) and MSN^{a}/siRNA-cy5/pD or Nanosac (red) at 24 h from IV injection. Scale bars: 50 μm. See FIG. 33 for additional micrographs. (FIG. 5F) quantitative analysis of micrographs in (FIG. 5E): % NPs departing from the lectin-positive endothelial cells was calculated as the area of free NPs (red) divided by the area of the total NP fluorescence (free NPs and NPs overlapping with endothelial cells: red+yellow). Three fields were randomly selected and analyzed by the Nikon A1R confocal microscope analysis software. (FIG. 5G) Photomicrographs of hematoxylin and eosin (H&E)-stained liver and spleen sections. No significant lesions were observed in either organ microscopically examined in all treatment groups. See FIGS. 34 and 35 for high magnification photomicrographs.

FIG. 6. Anti-tumor activity of D5W, anti-PD-L1 antibody, and Nanosac in Balb/c mice bearing CT26 tumors (anti-PD-L1 antibody: 200 μg/mouse/time, intraperitoneal injection; siPD-L1: 1.5 mg/kg/time, IV injection; q2dx5). *: p<0.05, **: p<0.01, and ***: p<0.001 between average tumor sizes on day 20 post-first injection by Tukey's multiple comparisons test following two-way repeated measures ANOVA. n=5 mice per group (mean±s.d).

FIG. 7. TEM images of Nanosac and the precursors. Negatively stained with 1% phosphotungstic acid. Scale bars: 50 nm.

FIG. 8 Functionalization scheme of MSN to MSN^{a} [MSN conjugated with APTES (3-aminopropyltriethoxysilane)].

FIG. 9. Gel electrophoresis of siLuc and siLuc incubated in 2M HF/8M NH₄F for 5 min. Lane 1: siLuc (100 μM), Lane 2: siLuc+2M HF/8M NH₄F (siLuc: 100 μM), Lane 3: Lane 1× 1/10 (siLuc: 10 μM), Lane 4: Lane 2× 1/10 (siLuc: 10 μM).

FIG. 10A-10B. Size distribution of (10A) MSN^{a}/siRNA/pD and Nanosac before (−) and after (+) incubation in 50% FBS for 2 h and (10B) MSN^{a}/siRNA/pD before (−) and after (+) incubation in 50% FBS for 24 h at 37° C. (n=3 tests of a representative batch, mean±s.d.)

FIG. 11. Schematic overview of flocculation and deflocculation of O/siRNA/pD (Nanosac). Flocculation occurs with the addition of etching solution (2M HF/8M NH₄F) to MSN^{a}/siRNA/pD. Nanosac is deflocculated after repeated rinsing with DW and D5W accompanied by gentle sonication.

FIGS. 12A-12B. (12A) Z-average and (12B) zeta potential of NPs through core etching and washing processes.

FIGS. 13A-13B. (13A) TEM images and particle sizes of Nanosac lyophilized in varying Nanosac:trehalose (w:w) ratios. (13B) Gel electrophoresis of Nanosac, fresh or lyophilized with trehalose, demonstrating the stability of siRNA encapsulation. The diffuse bands indicated by the red arrow in the visible image panel correspond to the gel loading dye.

FIGS. 14A-14B. (14A) Cytotoxicity of MSN^{a}, MSN^{a}/pD, and Nanosac on CT26 cells. (14B) Cytotoxicity of MSN^{a} compared with polyethyleneimine (PEI) (n=3 tests of a representative batch, mean±s.d.). Two-way ANOVA followed by Dunnett's multiple comparisons test. At all concentrations, NPs showed no difference from non-treated control.

FIGS. 15A-15C. Gel electrophoresis of supernatants and pellets of MSN^{a}/siRNA (siGAPDH (15A), siLuc (15B), or siPD-L1 (15C)) complexes at various weight ratios of MSN^{a} to siRNA. 0/1 indicates siRNA only: “pellet” and “supernatant” appear in different intensities in (15A), where “pellet” shows 10 μM of siRNA, whereas “supernatant” shows 10 μM of siRNA diluted in the 100 μL of HEPES buffer.

FIG. 16. UV and visible image of gel electrophoresis of waste solution from each production step. Lane 1: control siRNA; Lane 2: dopamine solution; Lane 3: control siRNA; Lane 4: etching solution; Lane 5: 1^{st} wash; Lane 6: 2^{nd} wash; Lane 7: 3^{rd} wash. The diffuse bands indicated by the red arrow in the visible image panel correspond to the gel loading dye.

FIGS. 17A-17B. (17A) Gel electrophoresis and quantitative presentation of siRNA released from Nanosac in DW (pH 3) in 120 h. (17B) Stability of siRNA incubated in DW (pH 3) for 24 h or 72 h.

FIGS. 18A-18B. UV and visible image of gel electrophoresis of (18A) siLuc, MSN^{a}/siLuc, MSN^{a}/siLuc/pD, and Nanosac with/without RNase ±SDS challenge and (18B) siPD-L1, MSN^{a}/siPD-L1, MSN^{a}/siPD-L1/pD, and Nanosac with/without 50% FBS challenge. siRNA or NPs were challenged with 166 U/mL RNase for 15 min±8 mg/mL SDS for additional 2.5 h or 50% FBS for 1 h, both at 37° C., and analyzed by agarose gel electrophoresis. Dark signals in dashed boxes of visible images indicate MSN^{a}/siRNA/pD and Nanosac stuck in the wells, not releasing and subjecting the siRNA to degradation by the challenge conditions. The diffuse bands indicated by the red arrow in the visible image panel correspond to the gel loading dye. The bands indicated by the blue arrow in the visible image show only in samples containing FBS (corresponding disappearance of bands indicated by the red arrow), likely due to dye complexation with proteins in FBS.

FIG. 19. Silencing of PD-L1 expression in IFN-γ-activated CT26 cells by MSN^{a}/siPD-L1, MSN^{a}/siPD-L1/pD, and Nanosac, measured after 48 h treatment in complete medium. siPD-L1 concentration varied from 50 nM to 200 nM. (n=3 test of a representative batch, mean t s.d.). **: p<0.01; ***: p<0.001, ****: p<0.0001 by Tukey's multiple comparisons test following two-way ANOVA.

FIG. 20. Confocal microscope images of CT26 cells incubated with MSN^{a}/siGAPDH-cy3, MSN^{a}/siGAPDH-cy3/pD, and Nanosac. Green: Wheat Germ Agglutinin (cell membrane); Red: siGAPDH-cy3; Blue: Hoechst 33342 (nuclei). Scale bars: 20 μm.

FIG. 21. Synthesis schematic of cy5-labeled MSN^{a} (MSN^{a}-cy5).

FIG. 22. Fluorescence intensity of cy5 after incubation in DW, pH 3, and 2M HF/8M NH₄F for 5 min. n=3 tests of a representative batch (mean±s.d).

FIG. 23. Cytotoxicity of chlorpromazine, methyl-β-cyclodextrin, and amiloride on CT26 cells. CT26 cells were incubated with each inhibitor for 30 min. n=6 tests of a representative batch, mean±s.d. *: p<0.05; **: p<0.01; ***: p<0.001; ****: p<0.0001 vs 0 in each treatment by Dunnett's multiple comparisons test following two-way ANOVA. Arrows indicate the concentration used in the endocytosis inhibition study (FIG. 3a).

FIG. 24. Effects of endocytosis inhibitors on NP uptake in serum-free medium. CT26 cells were preincubated for 30 min with each inhibitor at a subtoxic concentration, followed by treatment of MSN^{a}-cy5/pD in serum-free medium for 2 h (n=3 tests of a representative batch, mean±s.d.). ***: p<0.001 vs 37° C. by Dunnett's multiple comparisons test following one-way ANOVA.

FIG. 25. (25A) Intracellular trafficking of Lipofectamine/siRNA-cy3, cy5-labeled MSN^{a}, MSN^{a}/pD, and Nanosac in CT26 cells; (25B) Fluorescence intensity profiles along the white lines in (a); (25C) Pearson's correlation coefficients indicating the degree of NP/lysosome colocalization in confocal images: R=1 (perfect colocalization), R=0 (no colocalization). n=5 tests of a representative batch (mean±s.d). ***: p<0.001; ****: p<0.0001; ns: not significant by Tukey's multiple comparisons test following one-way ANOVA. Green: Lysotracker (lysosome); Red: cy5-labeled NPs; Blue: Hoechst 33342 (nuclei).

FIG. 26. Gel electrophoresis of siRNA release kinetics from Nanosac, performed in different pH and H₂O₂ (100 μM) with constant agitation at 37° C. A representative image of 3 replicates.

FIGS. 27A-27D. Quantitative measurement and confocal microscope images of CT26 (27A), J774a.1 (27B), HUVEC (27C), and TNF-α activated HUVEC (27D) cells incubated with MSN^{a}-cy5/pD and Nanosac in. n=3 tests of a representative batch (mean±s.d). ***: p<0.001 and ****: p<0.0001 by Sidak's multiple comparisons test following two-way ANOVA. Green: Wheat Germ Agglutinin (cell membrane); Red: cy5-labeled NPs; Blue: Hoechst 33342 (nuclei). Scale bars: 50 μm.

FIGS. 28A-28C. (28A) Representative Trans-Epithelial Electrical Resistance (TEER) of HUVEC monolayer (n=3, mean±s.d.). The arrow indicates the time point NPs were added to the HUVEC layer. (28B) Schematic of transendothelial transport of NPs. (28C) The percentage of NP transport across the TNF-α-activated HUVEC monolayer (n=3 tests of a representative batch, mean±s.d.). *: p<0.05 and **: p<0.01 by Tukey's multiple comparisons test following two-way ANOVA. ns: not significant.

FIG. 29. Z-section images of CT26 tumor spheroids incubated with MSN^{a}-cy5/pD or Nanosac. Scale bars: 500 μm.

FIG. 30. Western blot of PD-L1 expression in CT26 tumors of Balb/c mice treated with D5W, MSN^{a}/siPD-L1/pD, or Nanosac (siPD-L1: 0.75 mg/kg/time, q2dx10). n=4 mice per group.

FIG. 31. Representative flow cytometric dot plots of CD8⁺ and CD4⁺ cells in tumor draining lymph nodes (TDLNs) of treated animals.

FIG. 32. Biodistribution of siRNA-cy5 delivered by MSN^{a}/siRNA-cy5/pD and Nanosac (siPD-L1-cy5: 0.75 mg/kg) in Balb/c mice bearing CT26 tumors, 24 h after IV injection. n=3 mice per treatment (mean±s.d).

FIG. 33. Fluorescence micrographs of tumor sections showing FITC-lectin-stained vessels (green) and MSN^{a}/siRNA-cy5/pD or Nanosac (red). Scale bar: 50 μm.

FIG. 34. Low (34A, 34C and 34E) and high (34B, 34D and 34F) magnification photomicrographs of hematoxylin and eosin (H&E)-stained liver sections. Arrowheads in (34D) and (34F) indicate clusters of NPs laden in Kupffer cells. (34G) In one of the MSN^{a}/siPD-L1/pD-treated animals, the liver section showed severe mixed infiltrates (dashed box) associated with abundant clusters of macrophages laden with NPs (arrowheads) in a periportal region with hepatic dropout and necrosis (asterisk).

FIGS. 35A-35F. Low (35A, 35C and 35E) and high (35B, 35D and 35F) magnification photomicrographs of H&E-stained spleen sections. White dashed boxes in (35C, 35E) show a less prominent zonal pattern due to increased macrophages and plasma cells in the white pulp. Arrowheads in (35D) and (35F) indicate macrophages laden with NPs.

FIGS. 36A-36C. Representative H&E images of peripancreatic (pancreas designated by asterisk) and perihepatic lymph nodes, D5W (36A); MSN^{a}/siPD-L1/pD (36B), and Nanosac (36C).

FIGS. 37A-3D. Anti-tumor activity of D5W, MSN^{a}/siCont/pD, MSN^{a}/siPD-L1/pD, and Nanosac in Balb/c mice bearing CT26 tumors (siPD-L1: 1.5 mg/kg/time, q2dx7). (37A) Average tumor size (mm³). *: p<0.05; **: p<0.01 between average tumor sizes on day 16 post-first injection by Tukey's multiple comparisons test following two-way repeated measures ANOVA. (37B) Average body weight (g). Arrowheads indicate times of treatment. n=5 mice per treatment (mean±s.d). (37C) Expression of PD-L1 in CT26 tumors. n=3 mice per group (mean±s.d). (37D) Photomicrographs of H&E-stained liver and spleen sections. No abnormalities were observed in either organ in all treatment groups. Liver: MSN^{a}/siCont/pD (3) and MSN^{a}/siPD-L1/pD (5) groups have abundant brown dots, which are clusters of NPs laden in Kupffer cells as shown in high magnification photos (arrowheads in 4, 6). Nanosac (7) group also shows brown dots, smaller and fewer than those of MSN^{a}/siCont/pD (3) and MSN^{a}/siPD-L1/pD (5). Nanosac also occupies Kupffer cells but in fewer clusters (arrowheads in 8). Spleen: MSN^{a}/siCont/pD (11), MSN^{a}/siPD-L1/pD (13), and Nanosac (15) groups show mild sinusoidal expansion. On high magnification, sinusoids are expanded by macrophages, often containing brown pigments (NPs) (arrowheads in 12, 14, and 16).

### DETAILED DESCRIPTION

While the concepts of the present disclosure are illustrated and described in detail in the figures and the description herein, results in the figures and their description are to be considered as exemplary and not restrictive in character; it being understood that only the illustrative embodiments are shown and described and that all changes and modifications that come within the spirit of the disclosure are desired to be protected.

As used herein, the following terms and phrases shall have the meanings set forth below. Unless defined otherwise, all technical and scientific terms used herein have the same meaning as commonly understood to one of ordinary skill in the art.

In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosages may be single or divided, and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” includes human and non-human animals such as companion animals (dogs and cats and the like) and livestock animals. Livestock animals are animals raised for food production. The patient to be treated is preferably a mammal, in particular a human being.

As disclosed herein, a small interfering RNA (siRNA) is a unique term referring to double-stranded RNA molecules with 20-25 base pairs, involved in RNA interference. Definitions to those other RNAs can be found at Zhang, P. et al., J. Integr. Bioinform. 2019 September; 16(3): 20190027.

In some illustrative embodiments, this present disclosure relates to a process for manufacturing soft, non-cationic nanocapsules, termed Nanosac, for in vivo delivery of a therapeutic compound (TC) comprising the steps of
a. preparing said therapeutic compound (TC) to be delivered;
b. optionally preparing an amine-modified mesoporous silica nanoparticle (MSN);
c. coating the MSN with said TC to afford TC-MSN;
d. coating the TC-MSN with polydopamine (pD) to afford pD-TC-MSN; and
e. dispersing the pD-TC-MSN in a buffered oxide etch solution to remove MSN and affording said Nanosac with said therapeutic compound (TC).

In some illustrative embodiments, this present disclosure relates to a process for manufacturing soft, non-cationic nanocapsules, termed Nanosac, for in vivo delivery of a therapeutic compound (TC) as disclosed herein, wherein said TC is a small molecule drug or a biologic.

In some illustrative embodiments, this present disclosure relates to a process for manufacturing soft, non-cationic nanocapsules, termed Nanosac, for in vivo delivery of a therapeutic compound (TC) as disclosed herein, wherein said small molecule drug comprises paclitaxel, sorafenib, itraconazole, docetaxel, doxorubicin, bortezomib, carfilzomib, camptothecin, cisplatin, oxaliplatin, cytarabine, vincristine, irinotecan, amphotericin B, and gemcitabine.

In some illustrative embodiments, this present disclosure relates to a process for manufacturing soft, non-cationic nanocapsules, termed Nanosac, for in vivo delivery of a therapeutic compound (TC) as disclosed herein, wherein said biologic comprises antibody therapeutics, peptide therapeutics, protein therapeutics, therapeutic RNAs, therapeutic DNAs, and therapeutic enzymes.

In some illustrative embodiments, this present disclosure relates to a process for manufacturing soft, non-cationic nanocapsules, termed Nanosac, for in vivo delivery of a therapeutic compound (TC) as disclosed herein, wherein said biologic is a small interfering RNA (siRNA).

In some illustrative embodiments, this present disclosure relates to a process for manufacturing soft, non-cationic nanocapsules, termed Nanosac, for in vivo delivery of a therapeutic compound (TC) as disclosed herein, wherein said biologic is a cytokine selected from the group consisting of interleukin-2 (IL-2), interferon-α (IFN-α), IL-15, IL-21, and IL-12.

In some illustrative embodiments, this present disclosure relates to a process for manufacturing soft, non-cationic nanocapsules, termed Nanosac, for in vivo delivery of a therapeutic compound (TC) as disclosed herein, wherein said biologic is an antibody selected from the group consisting of rituximab, trastuzumab, gemtuzumab ozogamicin, alemtuzumab, tositumomab, cetuximab, ibritumomab tiuxetan, bevacizumab, panitumumab, catumaxomab, ofatumumab, ipilimumab, and brentuximab vedoitin.

In some illustrative embodiments, this present disclosure relates to a process for manufacturing soft, non-cationic nanocapsules, termed Nanosac, for in vivo delivery of a therapeutic compound (TC) as disclosed herein, wherein said therapeutic RNA is a messenger RNA (mRNA).

In some illustrative embodiments, this present disclosure relates to a process for manufacturing soft, non-cationic nanocapsules, termed Nanosac, for in vivo delivery of a therapeutic compound (TC) as disclosed herein, wherein said therapeutic RNAs are non-coding RNAs selected from the group consisting of small-interfering RNAs (siRNAs), microRNAs (miRNAs), piwi-interacting RNAs (piRNAs), enhancer RNAs (eRNAs), long non-coding RNAs (lncRNAs), and circular RNA (circRNAs).

In some illustrative embodiments, this present disclosure relates to a process for manufacturing soft, non-cationic nanocapsules, termed Nanosac, for in vivo delivery of a therapeutic compound (TC) as disclosed herein, wherein said Nanosac is useful for systemic delivery of a therapeutic molecule selected from the group consisting of small molecular drugs, antibody therapeutics, peptide therapeutics, protein therapeutics, therapeutic enzymes, and therapeutic nucleic acids (DNAs, RNAs).

In some illustrative embodiments, this present disclosure relates to a process for manufacturing soft, non-cationic nanocapsules, termed Nanosac, for in vivo delivery of a therapeutic compound (TC) as disclosed herein, wherein said Nanosac is useful as a cancer treatment.

In some illustrative embodiments, this present disclosure relates to a process for manufacturing soft, non-cationic nanocapsules, termed Nanosac, for in vivo delivery of a therapeutic compound (TC) as disclosed herein, wherein said Nanosac is useful as a treatment for diseases caused by viral and bacterial infections.

In some illustrative embodiments, this present disclosure relates to a process for manufacturing soft, non-cationic nanocapsules, termed Nanosac, for in vivo delivery of a therapeutic compound (TC) as disclosed herein, wherein said Nanosac as a cancer therapy is administered systemically.

In some other illustrative embodiments, this present disclosure relates to a composition of soft, non-cationic nanocapsules, termed Nanosac, useful for in vivo delivery of a therapeutic compound (TC), manufactured according to the steps of
a. preparing said TC to be delivered;
b. preparing an amine modified mesoporous silica nanoparticle (MSN);
c. coating the MSN with said TC to afford TC-MSN;
d. coating the TC-MSN with polydopamine (pD) to afford pD-TC-MSN; and
e. dispersing the pD-TC-MSN in a buffered oxide etch solution to remove MSN and affording said Nanosac with said therapeutic compound (TC).

In some other illustrative embodiments, this present disclosure relates to a composition of soft, non-cationic nanocapsules, termed Nanosac, useful for in vivo delivery of a therapeutic compound (TC) as disclosed herein, wherein said TC is a small molecule drug or a biologic.

In some other illustrative embodiments, this present disclosure relates to a composition of soft, non-cationic nanocapsules, termed Nanosac, useful for in vivo delivery of a therapeutic compound (TC) as disclosed herein, wherein said small molecule drug comprises paclitaxel, sorafenib, itraconazole, docetaxel, doxorubicin, bortezomib, carfilzomib, camptothecin, cisplatin, oxaliplatin, cytarabine, vincristine, irinotecan, amphotericin B, and gemcitabine.

In some other illustrative embodiments, this present disclosure relates to a composition of soft, non-cationic nanocapsules, termed Nanosac, useful for in vivo delivery of a therapeutic compound (TC) as disclosed herein, wherein said biologic comprises antibody therapeutics, peptide therapeutics, protein therapeutics, therapeutic RNAs, therapeutic DNAs, therapeutic interfering RNAs, and therapeutic enzymes.

In some other illustrative embodiments, this present disclosure relates to a composition of soft, non-cationic nanocapsules, termed Nanosac, useful for in vivo delivery of a therapeutic compound (TC) as disclosed herein, wherein said biologic is an interfering RNA (siRNA).

In some other illustrative embodiments, this present disclosure relates to a composition of soft, non-cationic nanocapsules, termed Nanosac, useful for in vivo delivery of a therapeutic compound (TC) as disclosed herein, wherein said biologic is a small interfering RNA (siRNA).

In some other illustrative embodiments, this present disclosure relates to a composition of soft, non-cationic nanocapsules, termed Nanosac, useful for in vivo delivery of a therapeutic compound (TC) as disclosed herein, wherein said Nanosac is useful for systemic delivery of a therapeutic treatment selected from the group consisting of small molecular drugs, antibody therapeutics, peptide therapeutics, protein therapeutics, therapeutic RNAs, therapeutic DNAs, therapeutic interfering RNAs, and therapeutic enzymes.

In some other illustrative embodiments, this present disclosure relates to a composition of soft, non-cationic nanocapsules, termed Nanosac, useful for in vivo delivery of a therapeutic compound (TC) as disclosed herein, wherein said composition is useful as a cancer treatment.

In some other illustrative embodiments, this present disclosure relates to a composition of soft, non-cationic nanocapsules, termed Nanosac, useful for in vivo delivery of a therapeutic compound (TC) as disclosed herein, wherein said composition as a therapy is administered systemically.

In some other illustrative embodiments, this present disclosure relates to a composition of soft, non-cationic nanocapsules, termed Nanosac, useful for in vivo delivery of a therapeutic compound (TC) as disclosed herein, wherein said composition is a pharmaceutical composition useful as a cancer treatment.

In some other illustrative embodiments, this present disclosure relates to a composition of soft, non-cationic nanocapsules, termed Nanosac, useful for in vivo delivery of a therapeutic compound (TC) as disclosed herein, wherein said Nanosac is useful as a treatment for diseases caused by viral and bacterial infections.

Yet in some other illustrative embodiments, this present disclosure relates to a method for treating a patient of cancer comprising the step of administering to a patient in need of relief from said cancer a therapeutically effective amount of a composition together with one or more diluents, excipients or carriers, wherein said composition is manufactured according to a process of:
a. preparing a therapeutic compound (TC) to be delivered;
b. preparing an amine modified mesoporous silica nanoparticle (MSN);
c. coating the MSN with said TC to afford TC-MSN;
d. coating the TC-MSN with polydopamine (pD) to afford pD-TC-MSN; and
e. dispersing the pD-TC-MSN in a buffered oxide etch solution to remove MSN and affording said Nanosac with said TC.

In some other illustrative embodiments, this present disclosure relates to a method for treating a patient of cancer as disclosed herein, wherein said TC is a small molecule drug or a biologic.

In some other illustrative embodiments, this present disclosure relates to a method for treating a patient of cancer as disclosed herein, wherein said small molecule drug comprises paclitaxel, sorafenib, itraconazole, docetaxel, doxorubicin, bortezomib, carfilzomib, camptothecin, cisplatin, oxaliplatin, cytarabine, vincristine, irinotecan, amphotericin B, and gemcitabine.

In some other illustrative embodiments, this present disclosure relates to a method for treating a patient of cancer as disclosed herein, wherein said biologic comprises antibody therapeutics, peptide therapeutics, protein therapeutics, therapeutic RNAs, therapeutic DNAs, therapeutic interfering RNAs, and therapeutic enzymes.

In some other illustrative embodiments, this present disclosure relates to a method for treating a patient of cancer as disclosed herein, wherein said biologic is a small interfering RNA (siRNA).

In some other illustrative embodiments, this present disclosure relates to a method for treating a patient of cancer as disclosed herein, wherein said pharmaceutical composition as a cancer therapy is administered systemically.

In some other illustrative embodiments, this present disclosure relates to a method for treating a patient of cancer as disclosed herein, wherein said Nanosac is useful as a treatment for diseases caused by viral and bacterial infections.

In some other illustrative embodiments, this present disclosure relates to a method for treating a patient of cancer as disclosed herein, wherein said Nanosac is useful for systemic delivery of a therapeutic treatment selected from the group consisting of small molecular drugs, antibody therapeutics, peptide therapeutics, protein therapeutics, therapeutic RNAs, therapeutic DNAs, therapeutic interfering RNAs, and therapeutic enzymes.

Most synthetic carriers of siRNA are cationic (15, 16). The positive charge allows the carrier to form an electrostatic complex with siRNA to protect it from nucleases and facilitate cellular uptake. However, it is also the main factor that makes it difficult to deliver siRNA systemically to non-RES targets. First, cationic carriers tend to interact with serum proteins non-specifically to form large aggregates that can cause embolism (17) or attract opsonins that subject them to phagocytic clearance (13, 18, 19). Moreover, cationic formulations often show intrinsic pro-inflammatory properties, leading to undesirable side effects, such as pulmonary inflammation (20-22). A stealth coating such as polyethylene glycol can help reduce non-specific protein interactions and attenuate the pro-inflammatory effects, however, at the expense of efficiency in cellular uptake (23) and endosomal escape of the particles (24, 25). Systemic delivery of siRNA to non-RES targets, including solid tumors, requires the carriers to circulate stably and reach target organs as intact particles, without causing toxicity and compromising the ability to load, protect siRNA and bring it to cells.

Another issue in nanocarrier-based siRNA delivery to tumors, which is common to most NPs, is the low tumor distribution (26). With poorly organized vasculature and dysfunctional lymphatics, tumors develop greater interstitial fluid pressure than normal tissues, which hinders transvascular convection and intratumoral penetration of a drug (27, 28). The compact extracellular matrix of the tumor microenvironment further limits the interstitial drug diffusion (29, 30). To improve the intratumoral delivery, a drug may be loaded in a carrier that is disintegrated in the acidity of tumors (31) or transported by caveolae-mediated endocytosis and transcytosis in tumors (32, 33). Alternatively, a drug can be loaded in a flexible carrier that can be deformed during the paracellular transport (34-38). Recent studies report that soft extracellular vesicles prove superior to its rigid counterparts in intratumoral delivery of doxorubicin (39) and hollow nanocapsules with a flexible polysaccharide shell improve interstitial transport of subcutaneously injected mRNA vaccine to lymph nodes (40). However, these approaches are yet to be demonstrated in systemic delivery of siRNA.

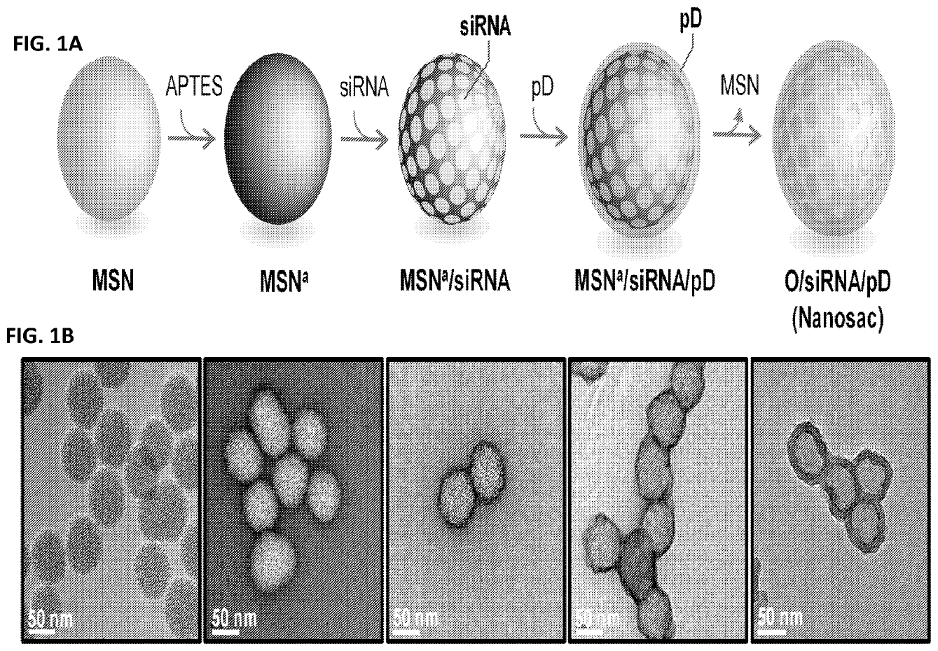

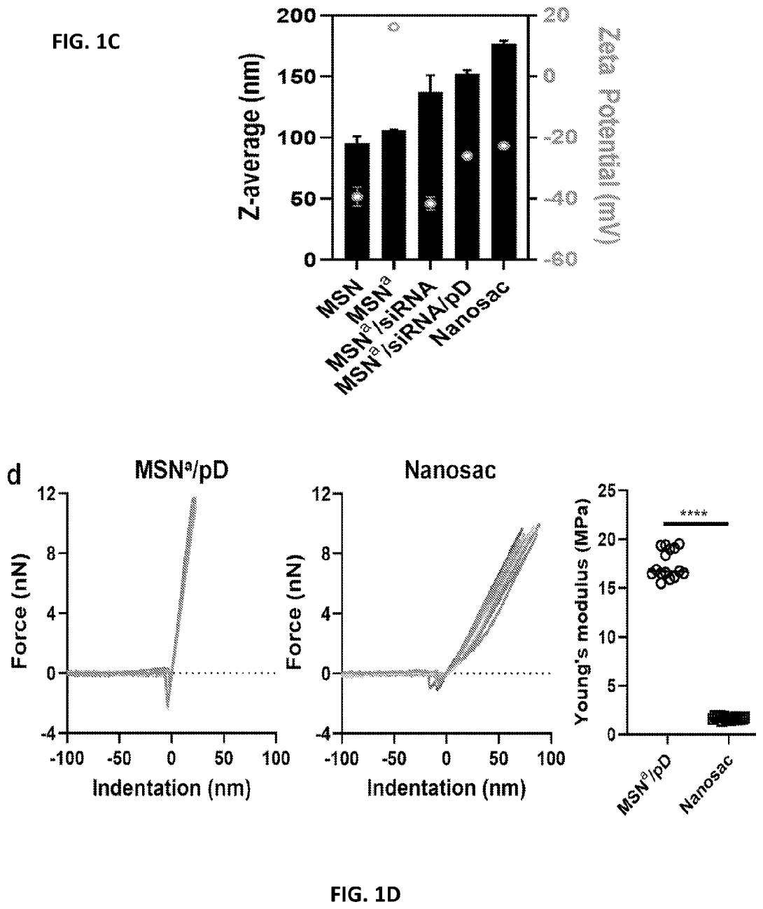

Given the challenges above, we aim to develop a new siRNA carrier for its systemic delivery to tumors. To avoid the issues related to cationic carriers and exploit the advantages of flexible systems, we produce soft and non-cationic nanocapsules, called Nanosac. siRNA is first coated on a sacrificial mesoporous silica nanoparticle (MSN) and covered with polydopamine (pD), whereupon the MSN core is removed to produce a hollow capsule (FIG. 1a). We hypothesize that Nanosac will protect siRNA in circulation and enhance transvascular and intratumoral delivery of siRNA. As a surface coating of NPs, pD has shown to recruit intact albumin in serum and facilitate transendothelial transport and cell uptake of the particles (41). Therefore, the pD capsule of Nanosac may provide additional benefits to systemic delivery of siRNA. To test these, we evaluate the stability of encapsulated siRNA, uptake pathway and intracellular trafficking, and extravasation and intratumoral transport of Nanosac. The efficiency of Nanosac as a systemic carrier of siRNA is demonstrated with a siRNA targeting immune checkpoint in a syngeneic mouse model of CT26 colorectal carcinoma.

Production of Nanosac

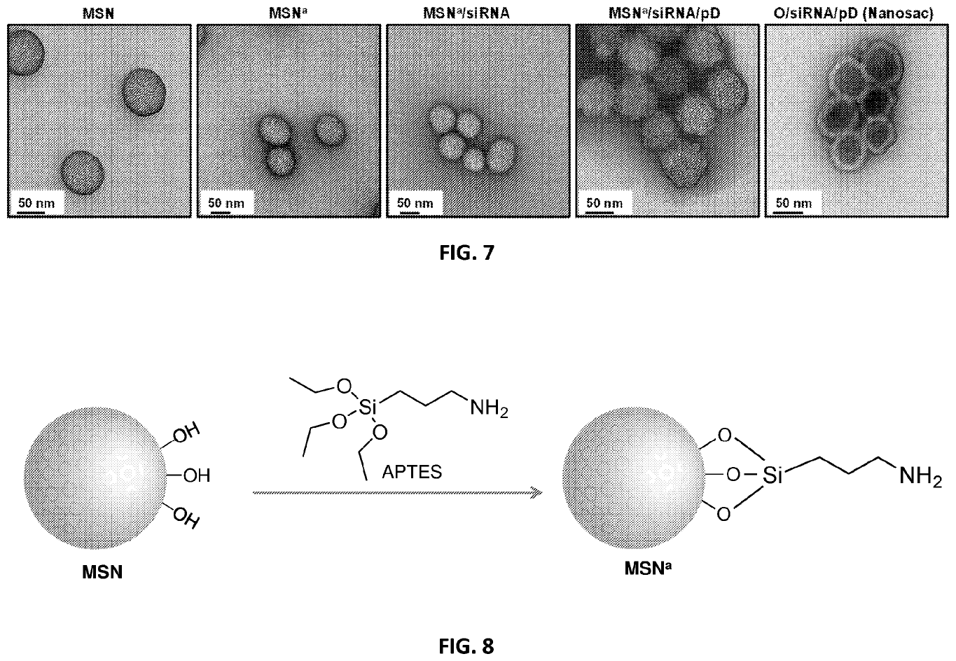

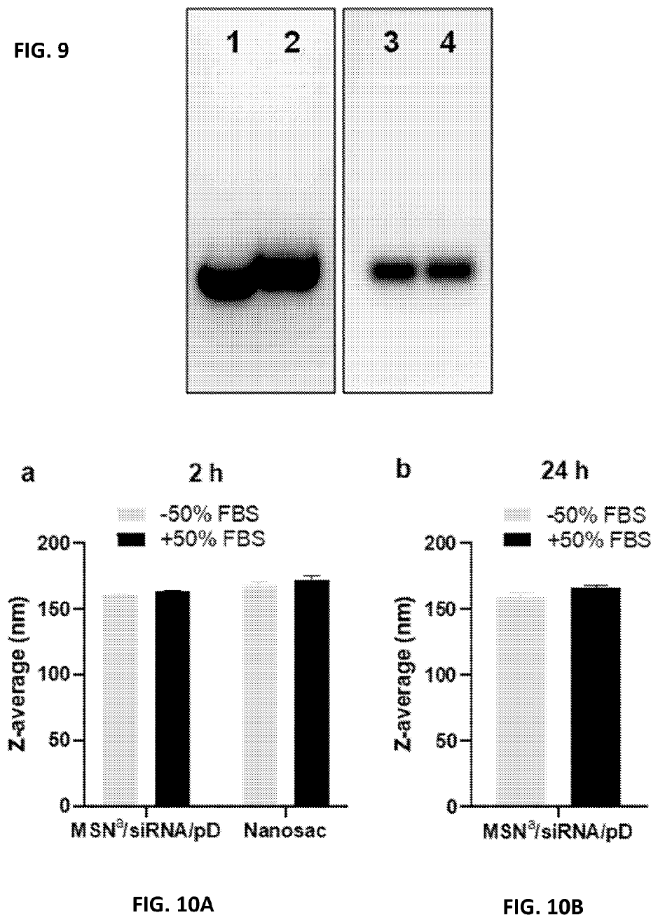

MSN was chosen as a sacrificial template due to the monodispersity, large surface area for siRNA loading, and the established protocol of removal. First, MSNs were synthesized by the sol/gel procedure (42). MSNs were spherical and negatively charged (−39.0±11.3 mV). The average diameter of MSNs was measured to be 67.2±2.7 nm by transmission electron microscopy (TEM) (FIG. 1B, FIG. 7) and 106.1±0.3 nm by dynamic light scattering (DLS), where the difference may be explained by a mild degree of MSN aggregation during the DLS measurement. Subsequently, the MSNs were modified with (3-aminopropyl)-triethoxysilane (APTES) to form amine-functionalized MSNs (MSN^{a}) (FIG. 8). The zeta potential of MSN^{a} was +16.2±5.9 mV due to the acquisition of amine groups on the surface. Negatively charged siRNA was adsorbed to MSN^{a} by electrostatic interaction. The siRNA-loaded MSN (MSN^{a}/siRNA) showed a negative charge (−41.6±2.1 mV), indicating the binding of siRNA on the MSN^{a} surface. After the siRNA binding, there was a slight increase in the hydrodynamic diameter (from 106.1±0.3 nm of MSN^{a} to 137.6±13.6 nm of MSN^{a}/siRNA) and the polydispersity index (PDI) (from 0.11 of MSN^{a} to 0.33 of MSN^{a}/siRNA), as measured by DLS. The slight size increase may be partly due to the addition of siRNA (1.7 wt % as described later); however, given no apparent change in NP morphology by TEM (FIG. 1B), the size increase may again be ascribed to mild aggregation during the siRNA encapsulation. Next, MSN^{a}/siRNA was coated with polymerized dopamine (pD) layer by 6 h incubation in dopamine solution. The pD-coated siRNA-bound MSN (MSN^{a}/siRNA/pD) showed brown color when dispersed in deionized water, a rough surface in TEM, a slight reduction of negative charge (from −41.6±2.1 mV of MSN^{a}/siRNA to −25.9±0.8 mV of MSN^{a}/siRNA/pD), and the increase of z-average (from 137.6±13.6 nm of MSN^{a}/siRNA to 152.3±2.7 nm of MSN^{a}/siRNA/pD), indicating the presence of pD shell. Lastly, siRNA-loaded nanocapsules (O/siRNA/pD, Nanosac) were produced by removing the MSN core from the MSN^{a}/siRNA/pD by hydrogen fluoride (HF) buffered to pH 5 with ammonium fluoride, in which siRNA was confirmed to be stable (FIG. 9). The resulting Nanosac showed a hollow nanocapsule structure with a z-average of 176.7±2.7 nm and a negative surface charge (−22.6±7.1 mV). The thickness of pD capsule, now clearly visible in TEM, is estimated to be 8.5±1.3 nm, which roughly corresponds to a half of the size difference (14.7 nm) between MSN^{a}/siRNA and MSN^{a}/siRNA/pD. In the absence of significant change in thickness, the increased size of Nanosac relative to MSN^{a}/siRNA/pD is thought to be a reflection of mild aggregation. FIG. 1c summarizes the zeta potentials and z-averages of all NPs measured by DLS (See also Table 1 below). Both MSN^{a}/siRNA/pD and Nanosac maintained their sizes in 50% fetal bovine serum (FBS) at least for 24 h (FIG. 10), indicating that it would circulate as nanoparticles without aggregation.

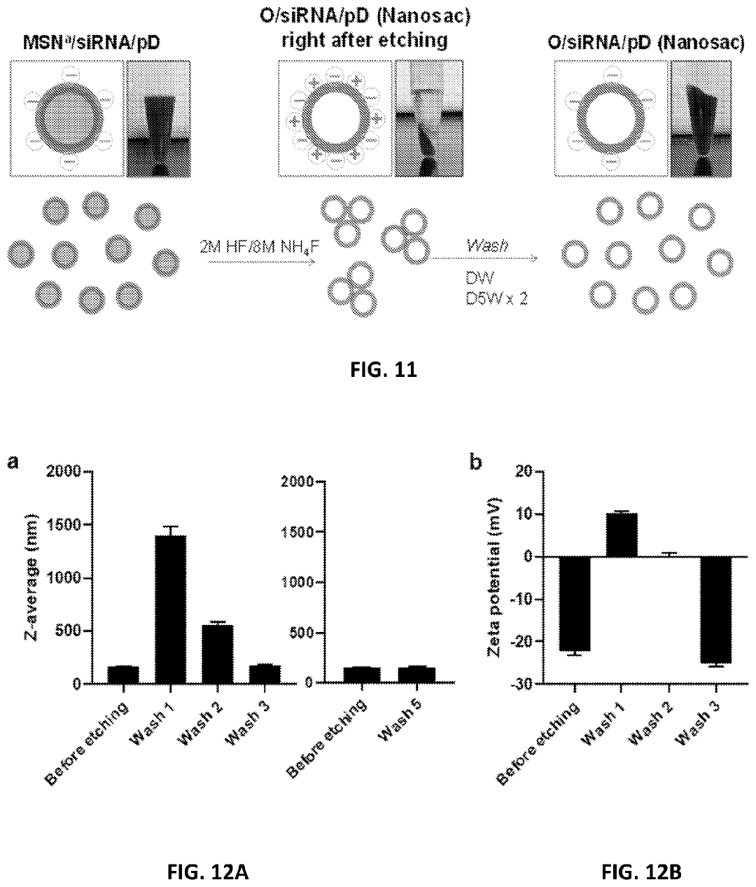

Of note, Nanosac could be collected and washed through low-speed centrifugation (2000 rcf), despite the small size. The ease of collection may be attributable to the formation of floccules, reversible aggregates of NPs (43-45), by charge neutralization of NP surface during the etching process (FIG. 11). After MSN^{a}/siRNA/pD was treated with cationic ammonium fluoride (2M HF/8M NH₄F, pH 5) for the removal of MSN, the NPs assumed a near neutral charge of 10.3±0.5 mV (in the first wash) and 0.027±0.91 mV (in the second wash), forming floccules with an average size of 1401.7±80.4 nm and 558.5±31.7 nm, respectively. NPs after the first wash seemed to have rather a positive charge, likely due to the residual free NH₄⁺ present at the slipping plane where the zeta potential is measured. With additional washing, the Nanosac floccules recovered the negative surface charge and the z-average of <200 nm (180.9 nm after the third wash; 157.1 nm after the fifth wash; FIG. 12).

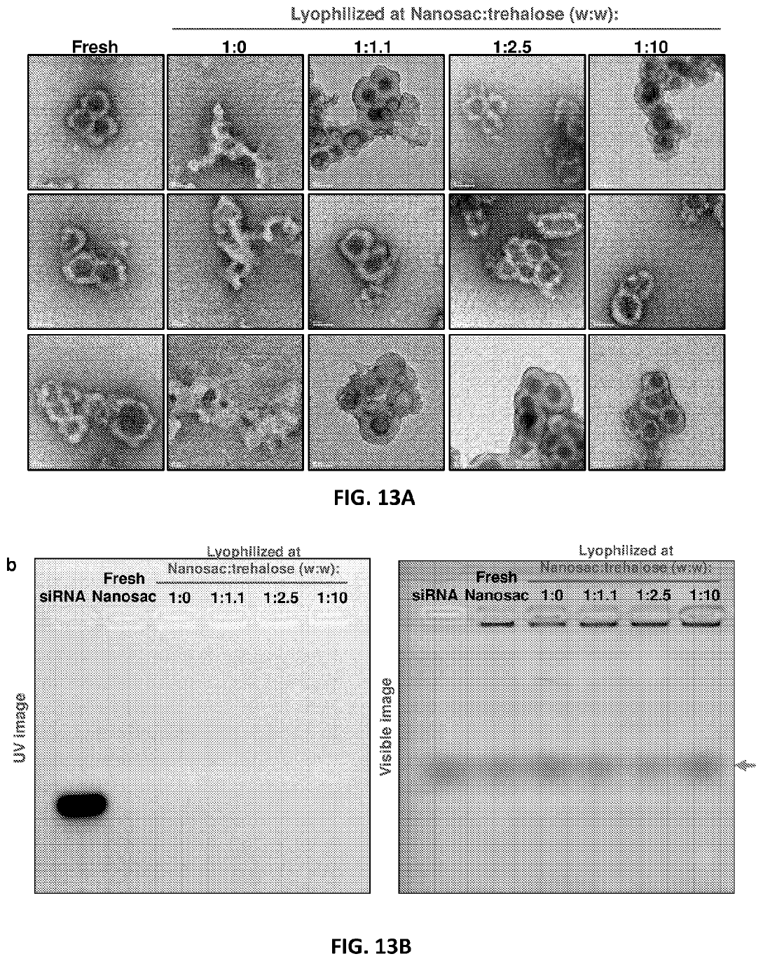

The feasibility of storing Nanosac as a lyophilized product was tested by freeze-drying Nanosac with a varying amount of trehalose as a lyoprotectant. Nanosac lyophilized with at as little as 110 wt % trehalose did not aggregate after lyophilization and maintained the particle size of freshly prepared Nanosac when reconstituted, whereas non-protected Nanosac aggregated to form >1 μm particles (FIG. 13a). The lyophilized Nanosac did not leak siRNA upon gel electrophoresis, indicating that Nanosac remained stable after lyophilization (FIG. 13b). While all the experiments in the current study were performed with freshly prepared NPs, these results suggest that Nanosac may be lyophilized and stored for later use.

Nanosac is softer than MSN^{a}/pD.

Nanosac, without the MSN core, was expected to be more flexible than the NP with the core. To determine the flexibility of NPs, we examined Young's moduli of MSN^{a}/pD and Nanosac by atomic force microscopy (AFM). The force-distance curve, a plot of the force measured by the AFM cantilever versus the distance between AFM tip and sample surface, was used to calculate the elastic moduli following the Hertzian model (46-48). MSN^{a}/pD and Nanosac showed a significant difference in the slope of the force-distance curve and the calculated Young's moduli (FIG. 1d). Nanosac exhibited a modulus of 1.6±0.2 MPa, ˜17-fold lower than that of MSN^{a}/pD (17.5±1.4 MPa), indicating that Nanosac is softer than MSN^{a}/pD. Of note, AFM was performed on dry NPs. The flexibility difference between the two NPs may be even greater in an aqueous medium, in which the Nanosac remain hydrated and swollen (48).

Nanosac is Non-Toxic In Vitro.

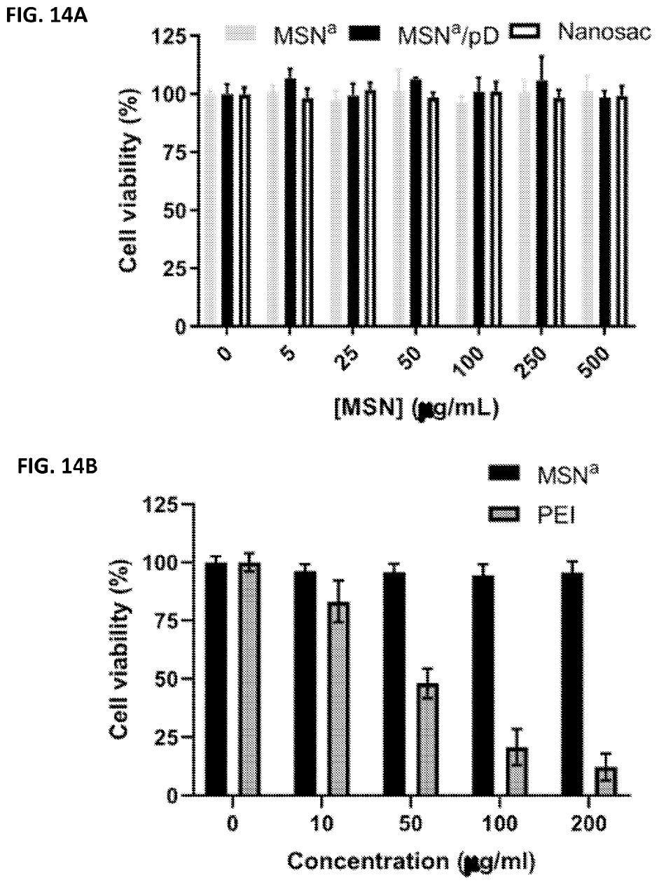

The cytotoxicity of MSN^{a}, MSN^{a}/pD, and Nanosac was examined with mouse colon carcinoma CT26 cells. After 48 h incubation with the cells, MSN^{a}, MSN^{a}/pD, and Nanosac showed negligible effects on cell viability at a concentration up to 500 μg/mL (higher concentration was not tested nor used) (FIG. 14a). In contrast, a common gene carrier polyethyleneimine showed dose-dependent toxicity in CT 26 cells, as expected of cationic polymers (FIG. 14b). Hence, the in vitro transfection of siRNA was carried out at 100 μg/mL, where the NPs were not apparently toxic.

Nanosac Encapsulates and Protects siRNA.

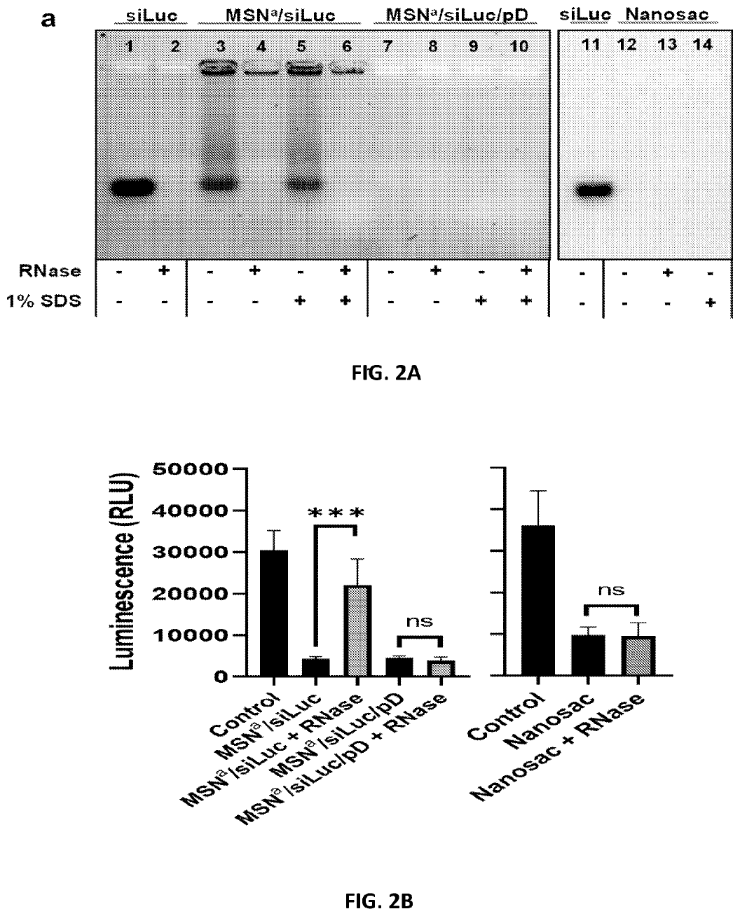

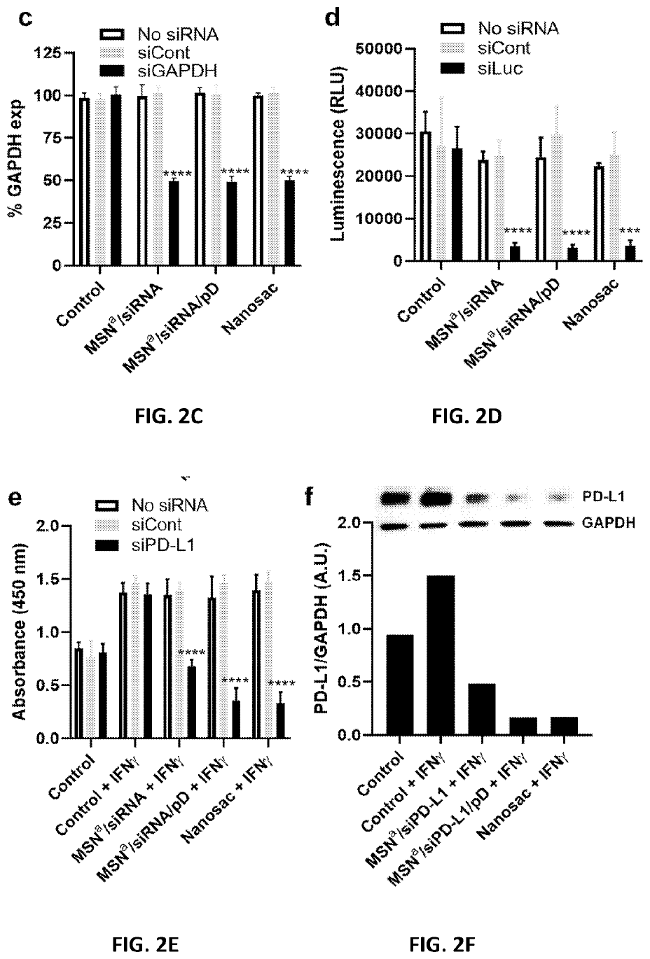

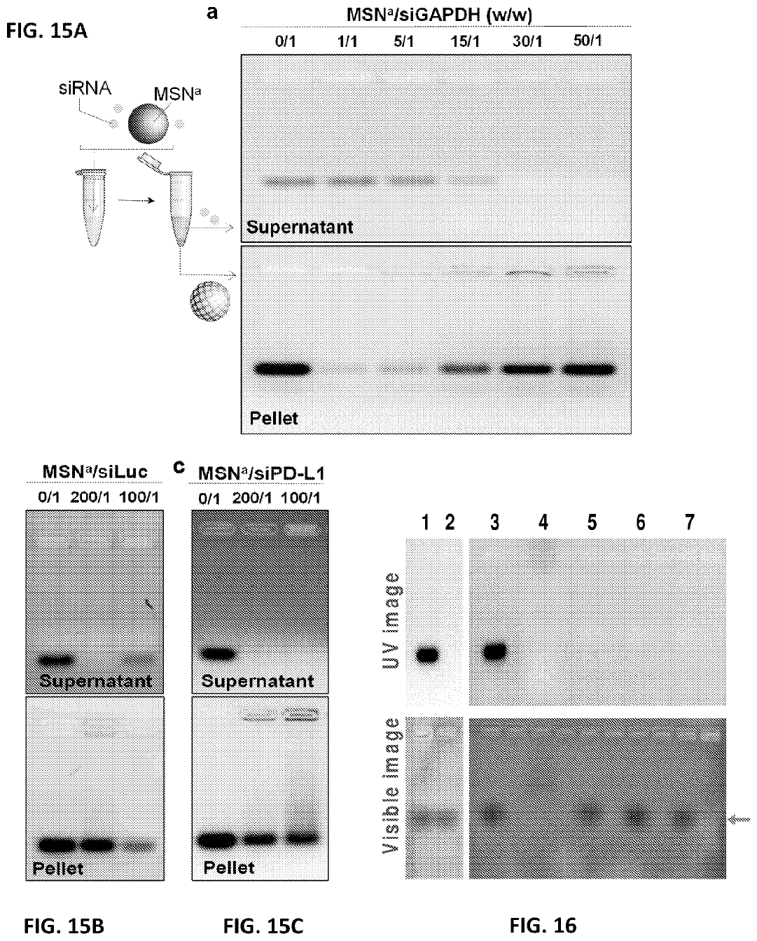

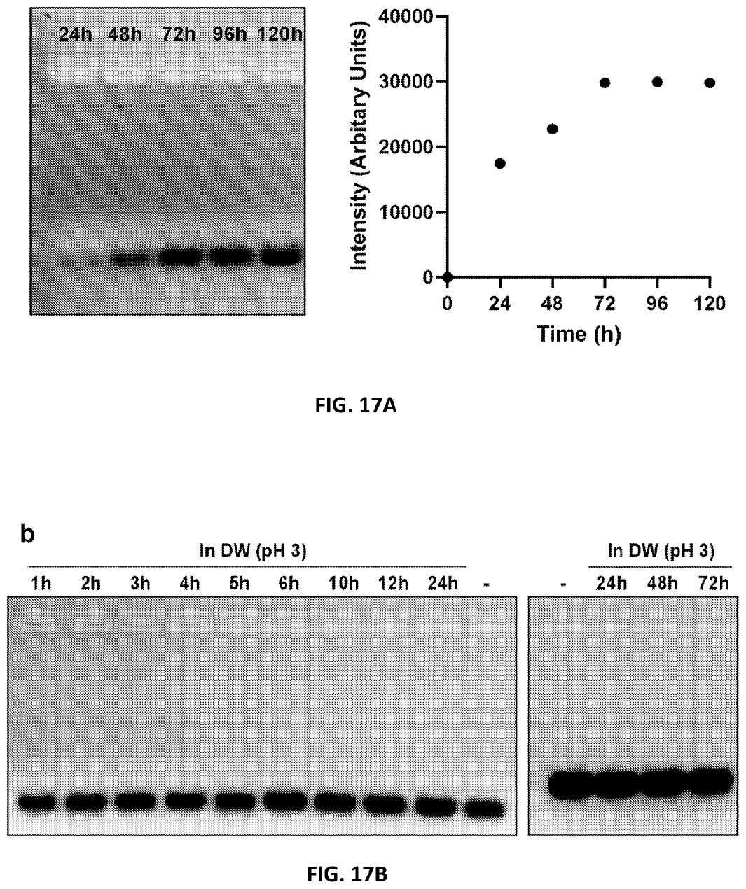

The siRNA binding capacity of MSN^{a} was evaluated by the agarose gel retardation assay. GAPDH-siRNA (siGAPDH) was complexed with MSN^{a} for 5 min, varying the weight ratio of siRNA to MSN^{a} (FIG. 15a). After the complexation, the supernatant (containing unbound siRNA) and the pellet (containing MSN^{a}/siGAPDH) were separated by centrifugation at 16,000 rcf and analyzed by agarose gel electrophoresis. The supernatant did not show free siRNA band at a MSN^{a}/siGAPDH weight ratio of 30 or higher (FIG. 15a top), suggesting that the siRNA binding capacity of MSN^{a} was as high as 3.3 wt %. Unlike typical polyplex or lipoplex, the MSN^{a}/siGAPDH complexes released siRNA upon gel electrophoresis (FIG. 9a bottom), indicating that the interaction between siRNA and MSN^{a} was relatively weak, consistent with an earlier study with aminated MSN and CpG oligodeoxynucleotide (49). siRNAs targeting luciferase gene (siLuc) and PD-L1 (siPD-L1) showed a similar pattern (FIG. 15b, c). On the other hand, MSN^{a}/siRNA/pD and Nanosac, which had additional pD layer on top of siRNA, showed no free siRNA band upon gel electrophoresis (FIG. 2a). This result demonstrates that the pD layer contributed to the stable encapsulation of siRNA. Consistently, we found no sign of siRNA loss during the pD coating, MSN etching and subsequent washing steps, further supporting the protective effect of the pD layer (FIG. 16). The quantity of siRNA loaded in Nanosac was directly measured by dissolving the pD layer and quantifying the siRNA eluted in gel electrophoresis. The pD layer was partially hydrolyzed in the acidic PBS (pH 3) for 72 h, the minimal time to achieve the maximum siRNA recovery (FIG. 17a). siRNA was stable in the acidic PBS for 72 h (FIG. 17b). The siRNA loading capacity of Nanosac (siRNA/Nanosac wt %) used in most studies was estimated to be 1.7 wt %.

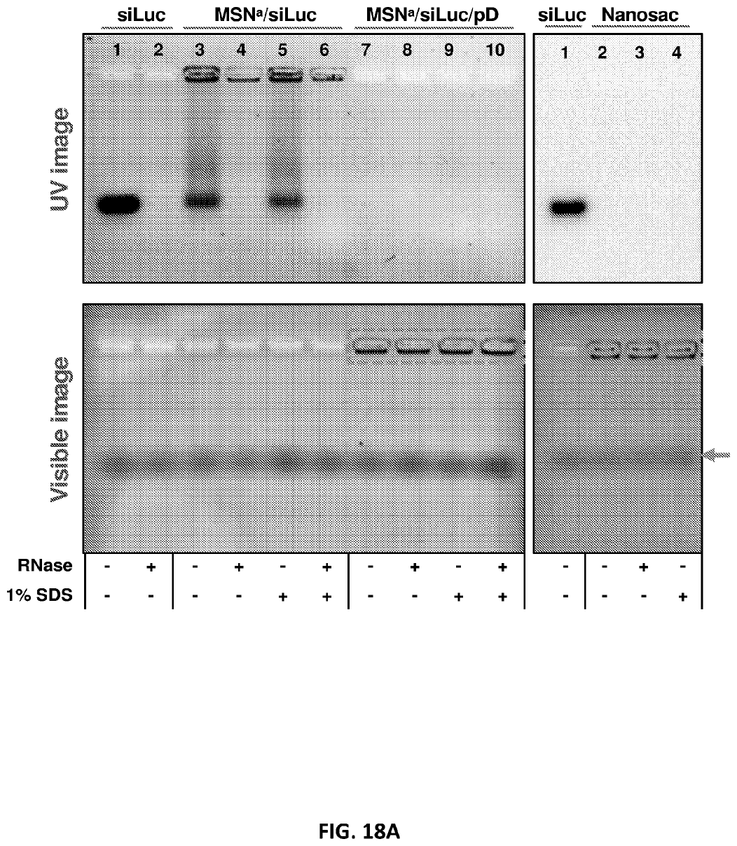

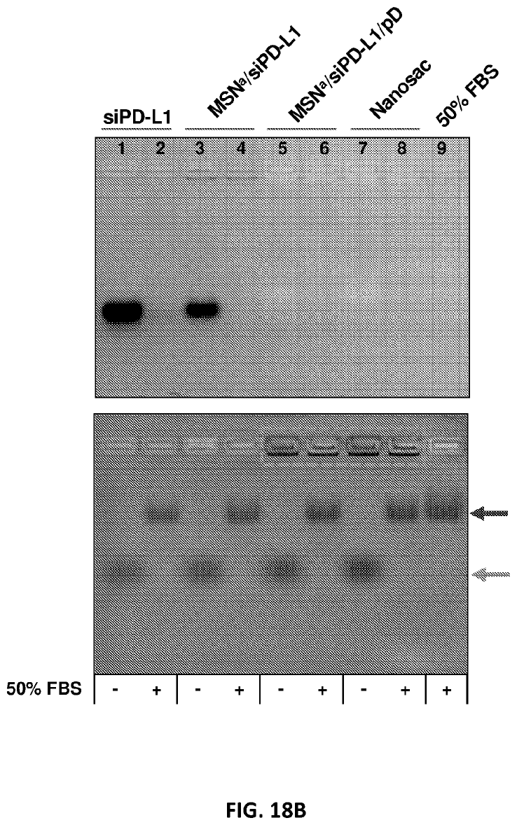

To test if the pD layer protects siRNA from enzymatic challenge and anionic environment in physiological fluids, MSN^{a}/siRNA, MSN^{a}/siRNA/pD, and Nanosac were subjected to ribonuclease A (RNase) 100 μg/mL±1% SDS (FIG. 2a) or 50% FBS. Free siLuc was completely degraded by RNase (FIG. 2a, Lane 2). MSN^{a}/siLuc, where siLuc was loosely bound to MSN^{a} (FIG. 15b; FIG. 2a, Lane 3), had a fraction of siLuc in the well (as a MSN^{a}/siLuc), but the loosely-bound siLuc was degraded by RNase treatment (FIG. 2a, Lane 4). Interestingly, 1% SDS-treated MSN^{a}/siLuc (FIG. 2a, Lanes 5 and 6) showed no additional signs of instability compared to SDS-non-treated MSN^{a}/siLuc (FIG. 2a, Lanes 3 and 4), indicating that electrostatic forces played only a partial role in the interaction between MSN^{a} and siLuc. This suggests that other forces less affected by 1% SDS, such as hydrogen bonding (50), contributed to the siRNA loading on MSN^{a}. Hydrogen bonding is attributable to —SiOH and —NH₂ of MSN^{a} and consistent with the loose binding (FIG. 15a bottom). On the other hand, MSN^{a}/siLuc/pD and Nanosac resisted RNase challenge ±1% SDS treatment, indicating that pD layer prevented the detachment and degradation of siRNA (FIG. 2a, Lanes 7-10). (The MSN^{a}/siLuc/pD was located in the gel well in the visible image of the gel, FIG. 18a). Consistently, MSN^{a}/siPD-L1/pD and Nanosac remained stable in 50% FBS, showing no signs of degradation due to serum nucleases, whereas siPD-L1 that was free or loosely bound to MSN^{a}/siPD-L1 was completely degraded (FIG. 18b). To verify the protection of siRNA from RNase by pD layer, we examined the in vitro transfection efficiency of MSN^{a}/siLuc, MSN^{a}/siLuc/pD, or Nanosac with and without RNase treatment (FIG. 2b). All NPs showed significant silencing of luciferase expression (14.2%, 14.8%, and 27.1% of the respective non-treated control) in 4T1-Luc cells. After RNase treatment, MSN^{a}/siLuc showed no significant silencing of the luciferase expression, whereas MSN^{a}/siLuc/pD and Nanosac maintained the silencing effect. This result is consistent with the gel electrophoresis and confirms that pD layer could protect siRNA from nuclease degradation. The protective effect of pD was preserved through the MSN removal process, as indicated by gel electrophoresis and gene silencing of the RNase-treated Nanosac.

Nanosac Silences Gene Expression In Vitro.

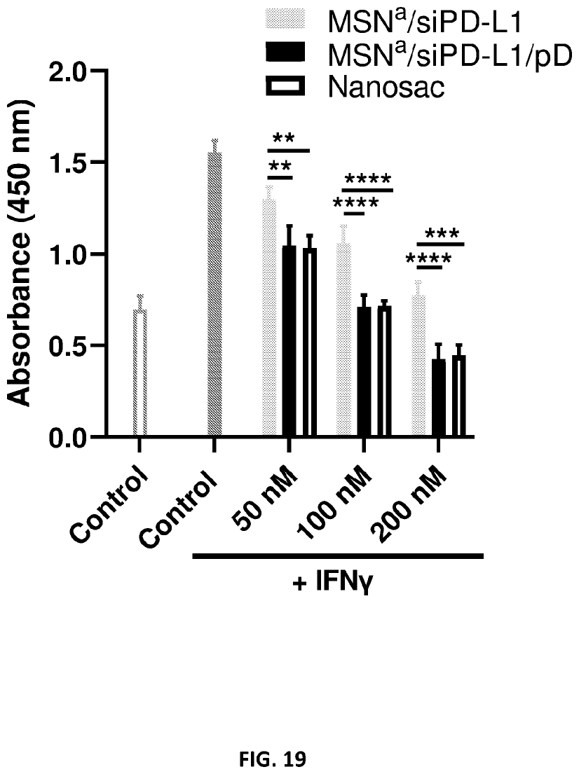

In vitro gene silencing by Nanosac and its precursors (MSN^{a}/siRNA, MSN^{a}/siRNA/pD) was evaluated with three siRNAs (siGAPDH, siLuc, and siPD-L1) with a non-specific siRNA (siCont) as a control. All NPs containing siGAPDH showed significant inhibition of GAPDH expression in CT26 cells, whereas those with siCont showed no difference from blank NPs (FIG. 2c). MSN^{a}/siGAPDH, MSN^{a}/siGAPDH/pD, and Nanosac reduced the GAPDH expression to 48.9, 48.9 and 49.4%, respectively, whereas the NPs with no siRNA or siCont induced no GAPDH silencing. A similar trend was observed with siLuc in 4T1-Luc cells (FIG. 2d). All NPs with siLuc exhibited excellent luciferase silencing effects compared to NPs with no siRNA or siCont. These results demonstrate that the additional pD layer and the absence of MSN core did not negatively affect siRNA delivery to the tested cells. In preparation for in vivo studies described later, the gene silencing effect was tested with siPD-L1 in CT26 cells. CT26 cells were incubated with 100 ng/mL of interferon-7 (IFN-γ), a condition that induces immunosuppression in tumor microenvironment (51, 52), to induce PD-L1 expression, prior to the NP treatment. MSN^{a}/siPD-L1, MSN^{a}/siPD-L1/pD, and Nanosac, at a concentration equivalent to 200 nM siPD-L1, reduced the PD-L1 expression by 50.5, 74.2, and 75.6% compared to blank NPs in the IFN-γ-treated CT26 cells, while those with siCont showed no silencing effect (FIG. 2e). A similar trend was shown in Western blots of the cell lysates (FIG. 2f). The silencing effect was dose-dependent. The cells treated with MSN^{a}/siPD-L1/pD and Nanosac at a concentration equivalent to 100 nM of siPD-L1 showed the same level of siPD-L1 expression as no-IFN-γ-treated control cells. At 200 nM siPD-L1, MSN^{a}/siPD-L1/pD and Nanosac further reduced siPD-L1 expression to below the basal level (FIG. 19). MSN^{a}/siPD-L1/pD and Nanosac showed greater silencing effects than MSN^{a}/siPD-L1 at all concentrations, suggesting a positive role of pD coating, including the protective effect shown in FIG. 2a. This difference was not evident with siGAPDH or siLuc. Given the dose-dependence, we speculate that siGAPDH or siLuc may have been more efficient than siPD-L1 and all three particles reached the maximum silencing effect at the dose tested in this study (100 nM for siGAPDH; 150 nM siLuc).

Nanosac Delivers siRNA to CT26 Cells Via Caveolae-Mediated Endocytosis.

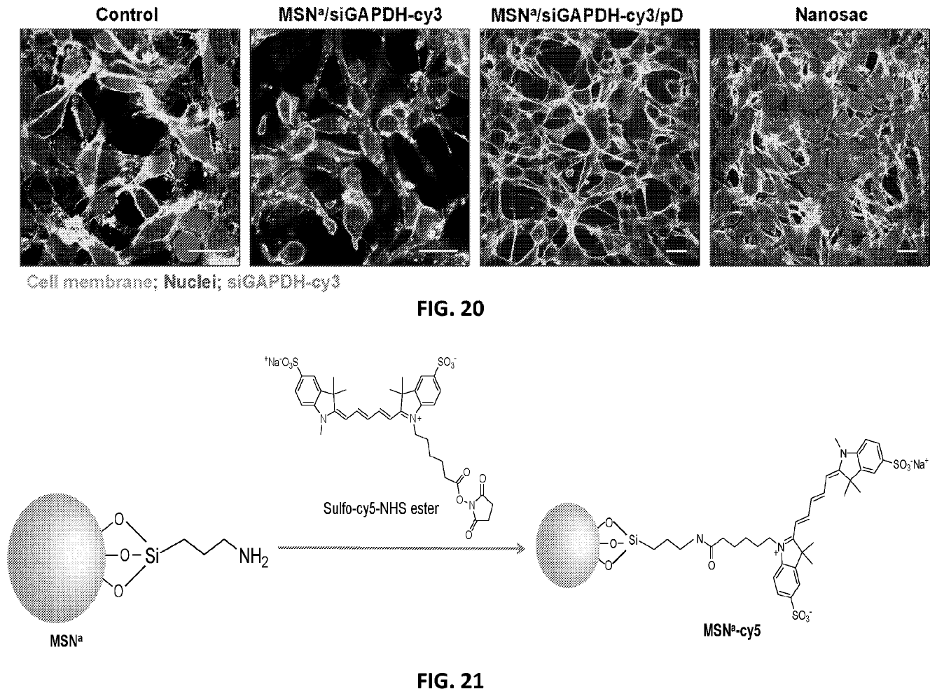

Gene silencing is the indirect evidence of efficient intracellular delivery of Nanosac. To verify intracellular delivery and understand the mechanism, we examined the uptake of NPs containing cy3-labeled siRNA (MSN^{a}/siRNA-cy3, MSN^{a}/siRNA-cy3/pD, and Nanosac) by CT26 cells by confocal microscopy. Consistent with gene silencing, all three NPs entered CT26 cells in 24 h (FIG. 20). Since none of the NPs has a specific ligand to facilitate their cellular uptake, we suspected that serum proteins in the medium adsorbed to the NPs forming a protein corona to mediate cellular uptake.

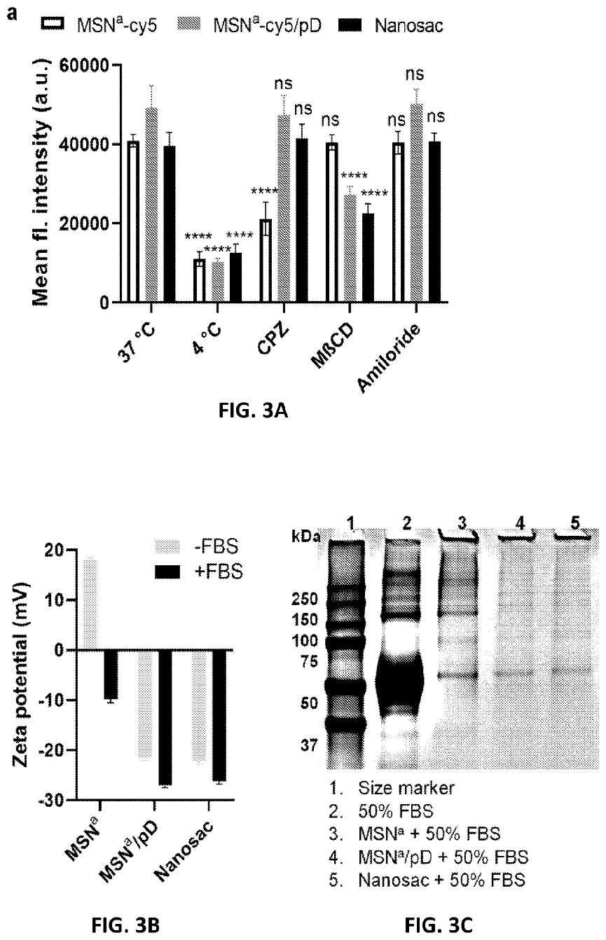

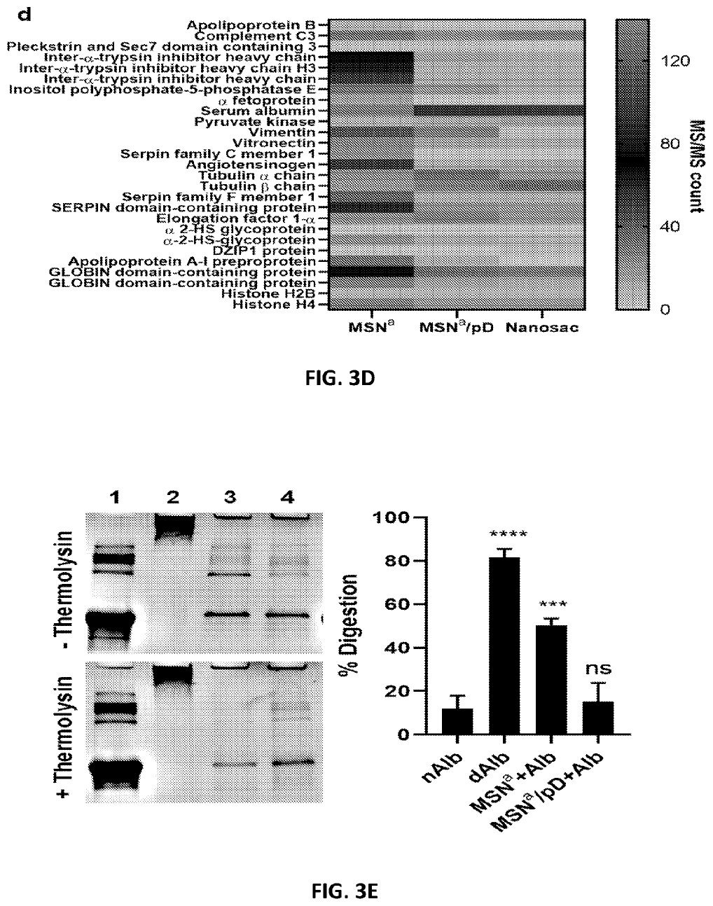

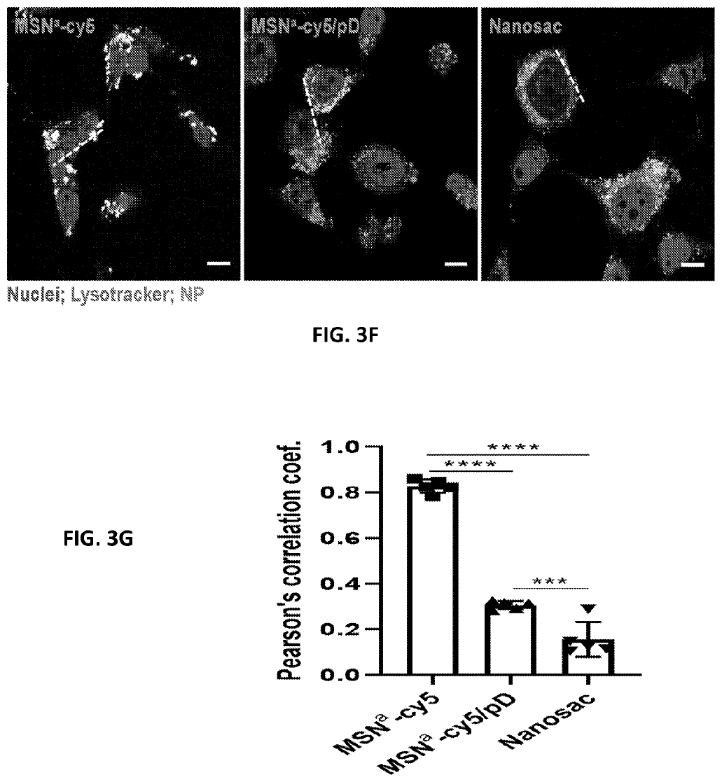

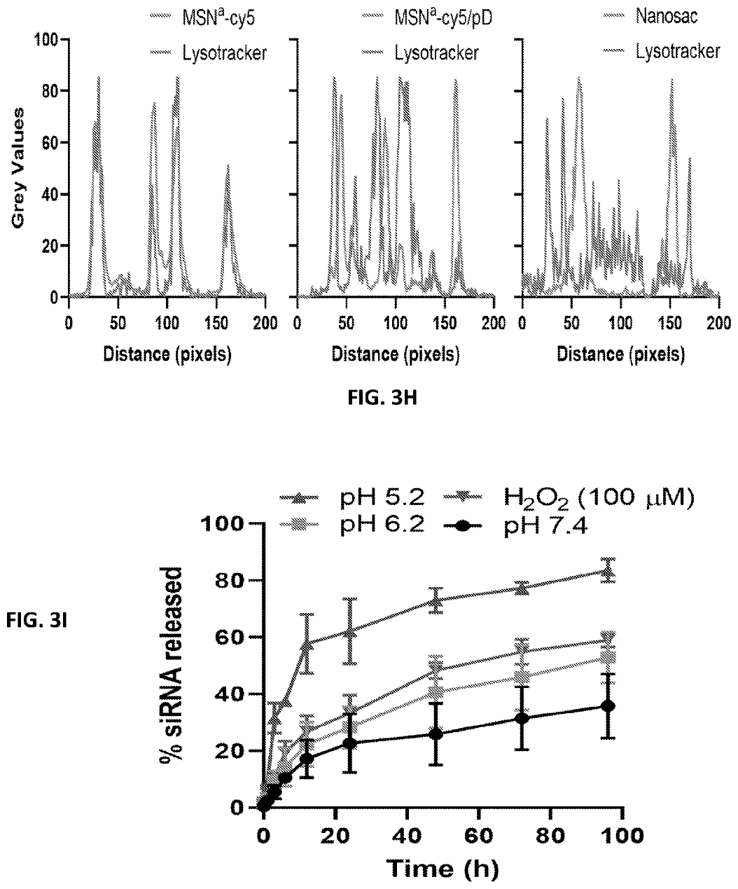

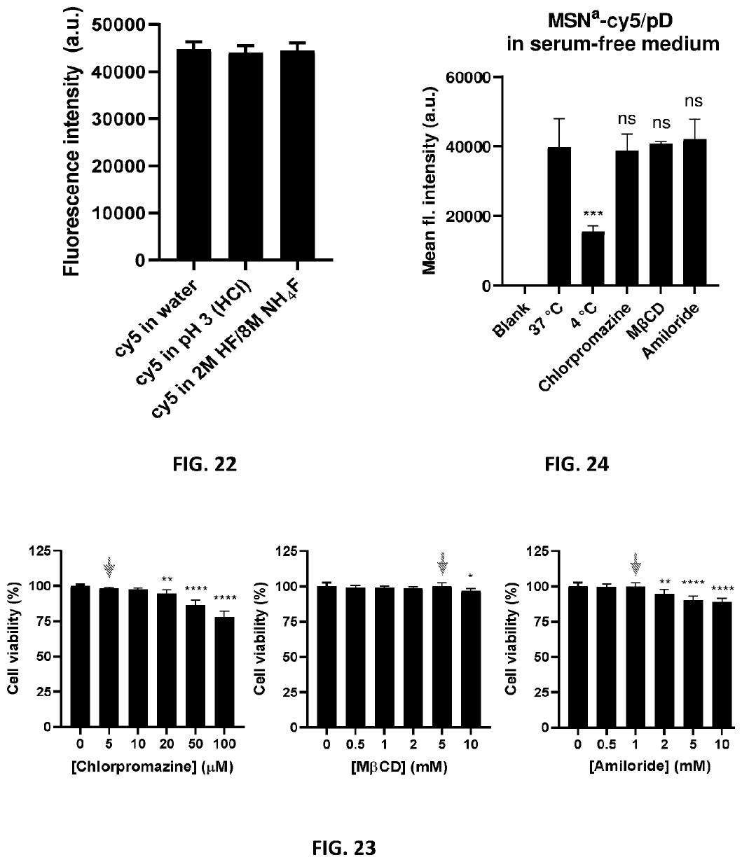

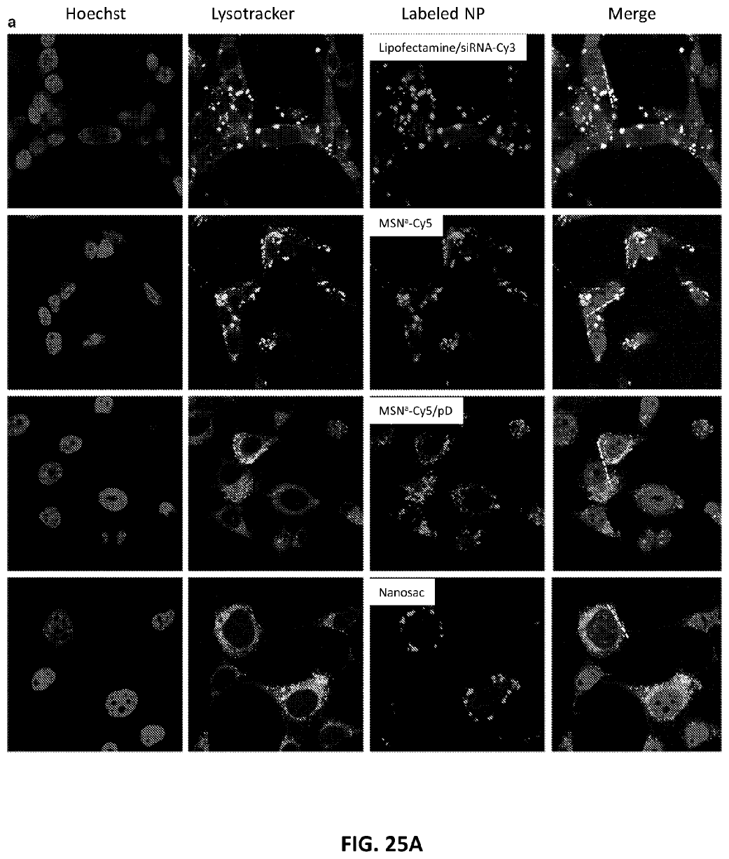

To test this, we incubated CT26 cells with the NPs under conditions blocking specific endocytosis pathways and analyzed by fluorospectrometry. MSN^{a} was first labeled with Cy5 (FIG. 21) for detection and quantification of uptake. The dye was confirmed to be stable through the dissolution of pD layer or the removal of MSN (FIG. 22). CT26 cells were incubated at 4° C. for 30 min to inhibit energy-dependent endocytosis or pretreated with inhibitors of endocytosis pathways, such as chlorpromazine (inhibitor of clathrin-mediated endocytosis), methyl-β-cyclodextrin (inhibitor of caveolae-mediated endocytosis), and amiloride hydrochloride (inhibitor of macropinocytosis) for 30 min at a subtoxic concentration for each compound (FIG. 23). The cellular uptake of all three NPs in serum-containing medium was blocked at 4° C. (FIG. 3a), which suggests that all NPs used energy-dependent internalization pathways. The uptake of MSN^{a}-cy5 (without pD) in serum-containing medium was significantly inhibited by chlorpromazine (CPZ), indicating that the naked MSN^{a} used a clathrin-mediated endocytosis pathway. In contrast, the uptake of pD-coated NPs (MSN^{a}-cy5/pD and Nanosac) was significantly inhibited by methyl-β-cyclodextrin, but not by chlorpromazine or amiloride hydrochloride, indicating that the pD-coated NPs were mainly internalized by the cells via caveolae-mediated endocytosis. These results suggest that pD layer recruits proteins in the serum-containing medium in a distinct manner than the naked MSN^{a} to enable caveolae-mediated endocytosis. In the serum-free medium, cellular uptake of MSN-cy5/pD was not affected by methyl-β-cyclodextrin (nor by other inhibitors) (FIG. 24), supporting the putative role of serum proteins in endocytosis of the pD-coated NPs. Of note, the NP uptake in serum-containing medium was not completely reduced by the tested endocytosis inhibitors to the same level as that in 4° C. (FIG. 3a), and MSN^{a}-cy5/pD uptake in serum-free medium was still affected by the low temperature (FIG. 24). This suggests that the NPs may also enter CT26 cells by additional pathways, such as clathrin-/caveolae-independent endocytosis (53), irrespective of the protein corona.

Albumin Binding to Nanosac Mediates its Cellular Uptake.

To identify the serum proteins responsible for differential cellular uptake profiles, we incubated MSN^{a} and pD-coated NPs (MSN^{a}/pD and Nanosac) with 50% FBS, rinsed twice, and analyzed by SDS-PAGE and LC-MS/MS. All NPs showed a reduction of zeta potential upon the incubation with 50% FBS (FIG. 3b), reflecting protein corona formation. MSN^{a} displayed the most change, suggesting a relatively high protein binding per NP, which was confirmed by SDS-PAGE band intensity (FIG. 3c) and MS/MS counts (FIG. 3d). Albumin (66 kDa) binding was prominent for all NPs, whereas MSN^{a} showed additional protein binding (100-250 kDa). LC-MS/MS analysis verified that the albumin was the major corona protein for all NPs. The additional proteins bound to MSN^{a} included complement c3, inter-α-trypsin, vimentin, angiotensinogen, SERPIN domain-containing protein, and GLOBIN domain-containing protein. The differential pattern of protein corona may be one explanation for the difference between MSN^{a} and the pD-coated NPs in cellular uptake profile. Since our previous study showed that the conformation of surface-bound albumin could also affect the endocytosis pathway (41), we examined the status of albumin bound on MSN^{a} and MSN^{a}/pD by the thermolysin proteolysis, which digests unfolded proteins (FIG. 3e). The band intensity of denatured albumin and the albumin bound to MSN^{a}-cy5 reduced by 81.5±4.1% and 50.4±3.0%, respectively, upon the pulse proteolysis, whereas native albumin underwent 12.1±5.8% of proteolysis. The albumin bound to MSN^{a}/pD showed a similar degree of digestion (15.2±8.6%) as native albumin. This result indicates that albumin conformation was better preserved on MSN^{a}/pD than on MSN^{a}.

Nanosac Traffics to Cytosol and Releases siRNA by ROS.

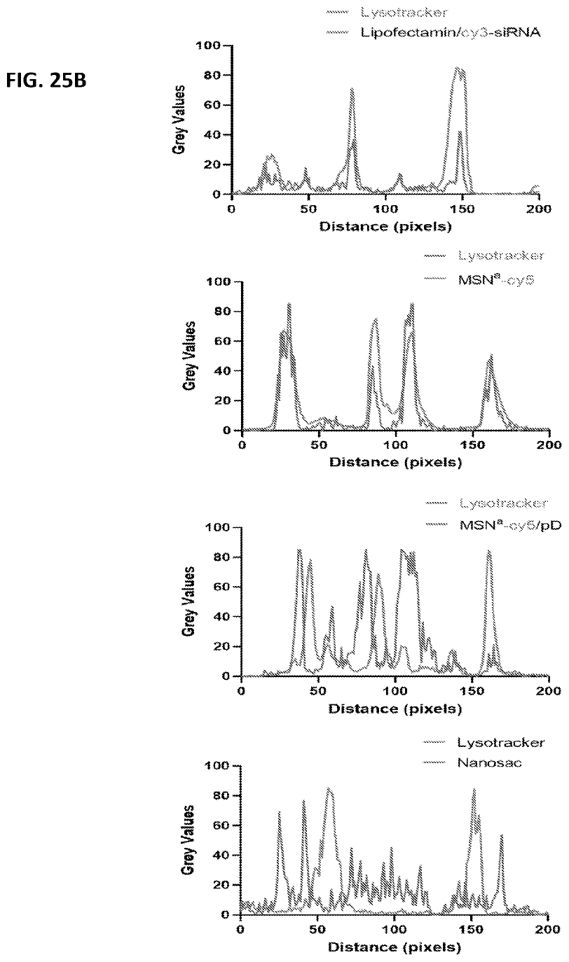

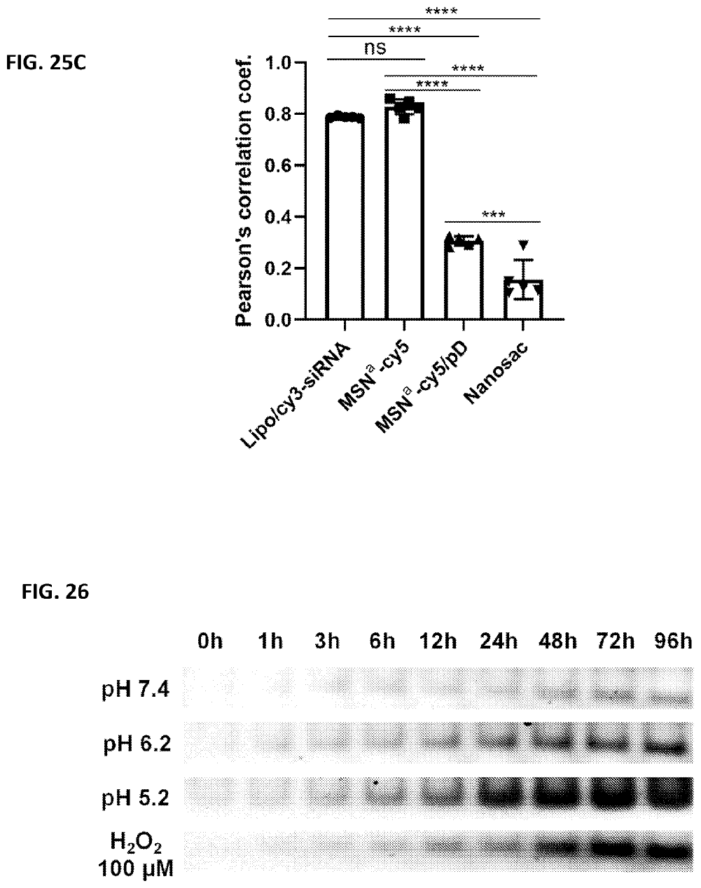

The results so far collectively suggest that the pD-coated NPs enter CT26 cells via the caveolae-mediated pathway due to the surface-bound albumin, at least partly. To compare intracellular trafficking, we located Nanosac and its precursors in the cells relative to a lysosome marker (LysoTracker Green) (FIG. 3f). MSN^{a}-cy5 was colocalized with LysoTracker Green (FIG. 3h) with a Pearson's correlation coefficient of 0.83 (FIG. 3g), indicating a high degree of colocalization of MSN^{a}-cy5 and lysosomes, i.e., that most of the internalized MSN^{a}-cy5 was transported to lysosomes. This was comparable to siRNA-cy3 complexed with Lipofectamine 2000 (Lipofectamine/siRNA-cy3), a common transfection reagent known to enter cells by the clathrin-mediated endocytosis (FIG. 25). In contrast, only a few MSN^{a}-cy5/pD and Nanosac were overlapped with lysosomes (FIG. 3h) with a Pearson's correlation coefficient of 0.15-0.3 (FIG. 3g), showing that the caveolae-mediated endocytosis facilitated cytosolic delivery of the pD-coated NPs avoiding destructive lysosomal sequestration.

The pD layer of Nanosac degrades in acidic conditions releasing siRNA, as shown in the determination of siRNA loading (pH 3, FIG. 3i, FIG. 26) and release studies performed at pH 5.2 and 6.2. However, when Nanosac directly traffics to cytosol, the release of siRNA from Nanosac may not be explained by the acidic pH of lysosomes. Instead, we suspect that reactive oxygen species (ROS), known to be elevated in malignant cells (54), may facilitate intracellular release of siRNA. To test this, we incubated Nanosac in 100 μM H₂O₂ (pH 7.4) solution (55) at 37° C. with agitation. Nanosac released more siRNA in the presence of H₂O₂ (FIG. 3i, FIG. 26), suggesting that cytosolic ROS may account, at least partly, for the release of siRNA from the cytosol-trafficked Nanosac and subsequent gene silencing.

Softness of Nanosac Helps Avoid Macrophage Uptake without Affecting the Interactions with Tumor Cells or Endothelial Cells.

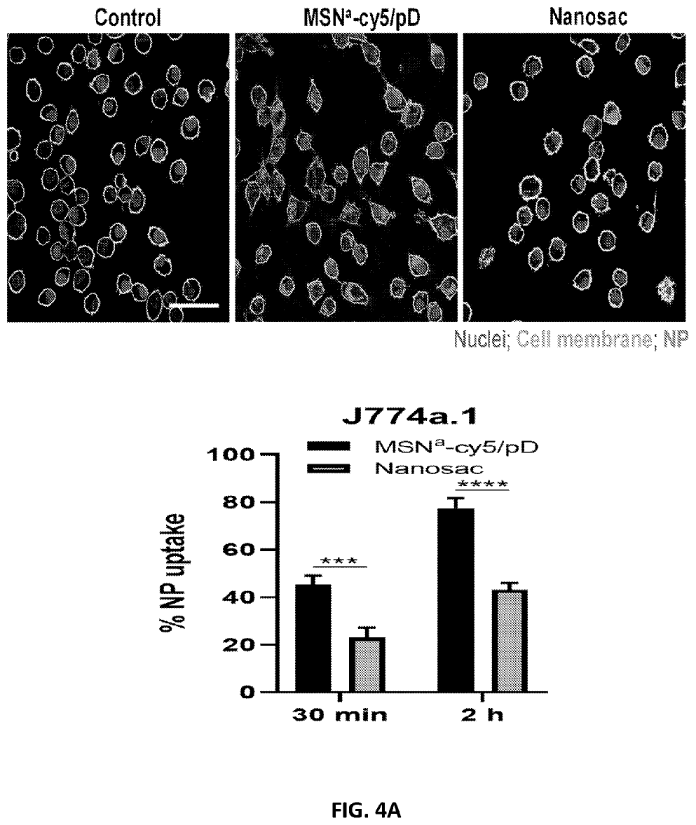

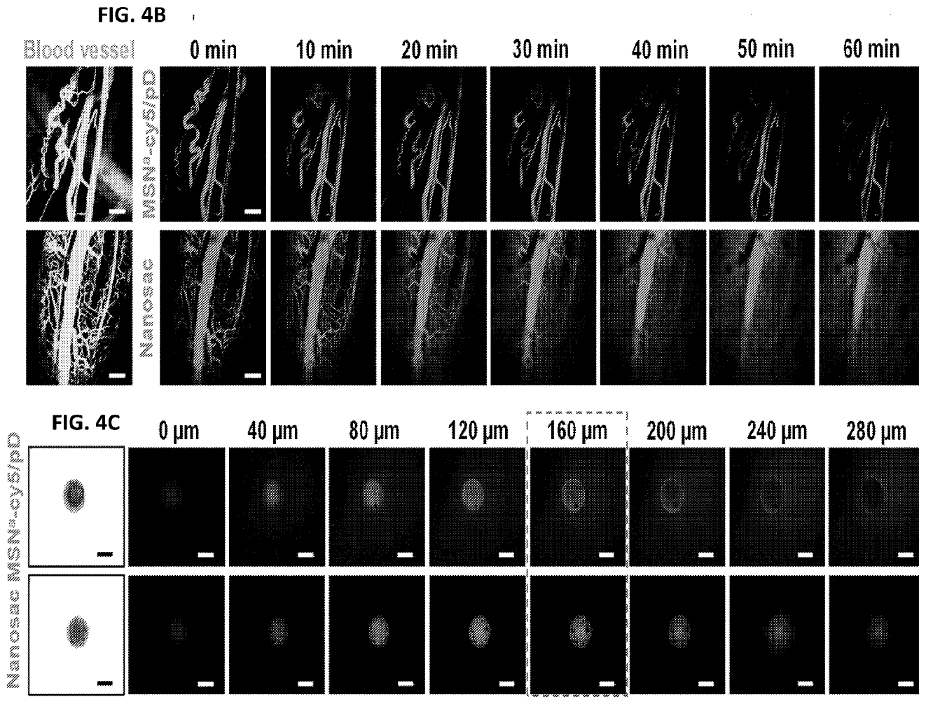

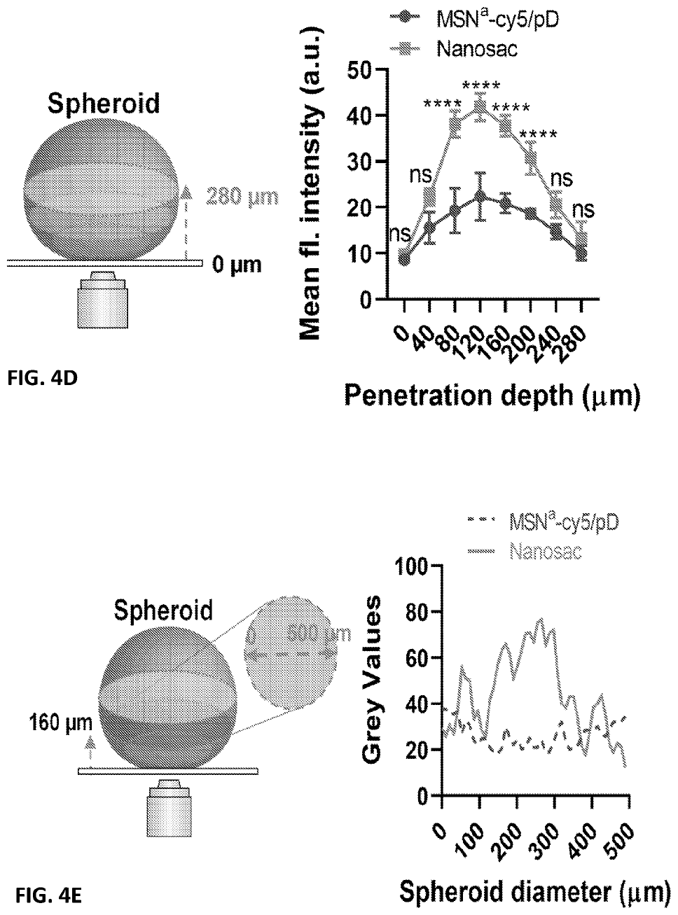

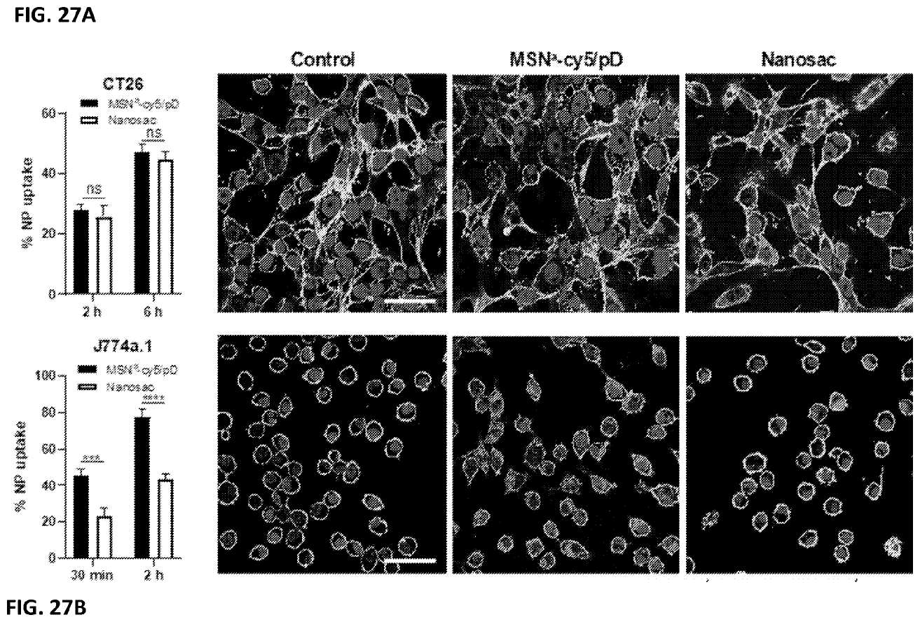

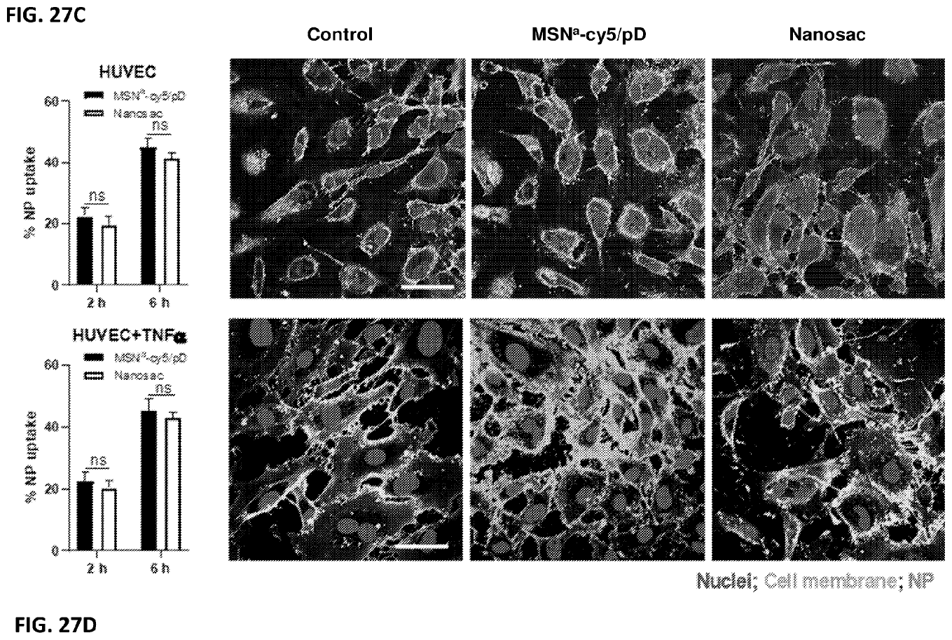

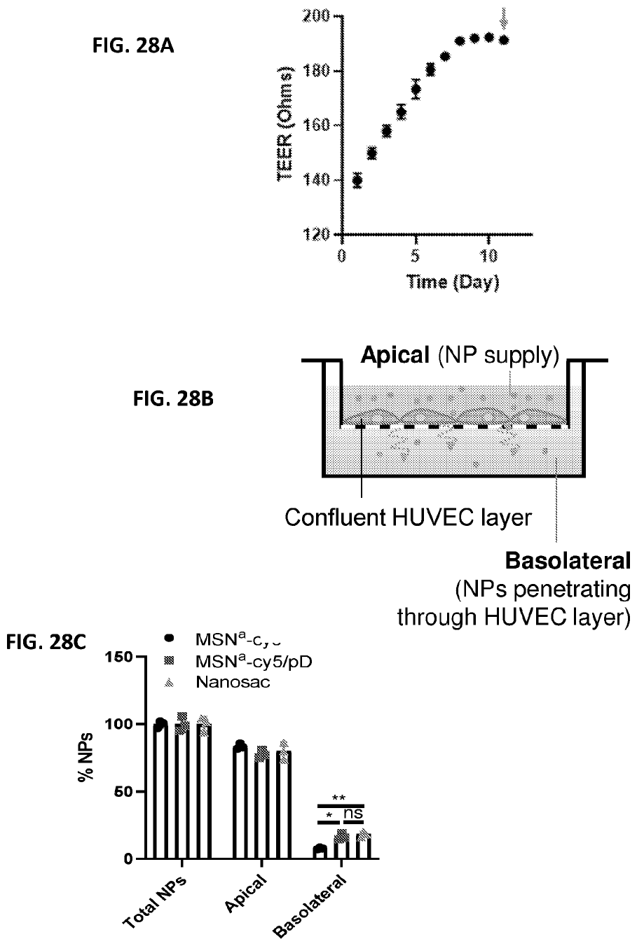

Systemically injected drug carriers first encounter immune cells or macrophages, which destroy therapeutic siRNA before reaching the target (56-60). Surviving carriers are translocated across the endothelial layer to enter tumors. Ideal drug carriers should avoid the recognition and internalization by macrophages but efficiently interact with peritumoral endothelium. We examined if MSN^{a}-cy5/pD and Nanosac, which entered CT26 cells similarly well (FIGS. 20, 27), show any difference in the uptake by macrophages and endothelial cells due to the differential flexibility (FIG. 1d). MSN^{a}-cy5/pD and Nanosac were incubated with J774A.1 macrophages and HUVECs. Unlike CT26 cells, macrophages took up Nanosac 2-fold less than MSN^{a}-cy5/pD in 30 min and 2 h (FIG. 4a), indicating that the softness of Nanosac helped avoid macrophage uptake. Confocal microscopy showed a consistent result. In contrast, MSN^{a}-cy5/pD and Nanosac entered HUVEC, both resting and activated by TNF-α (simulating peritumoral endothelium), with comparable efficiency (FIG. 27), as they did CT26 cells. We then examined if the NPs can translocate through the endothelial layer. NPs (MSN^{a}-cy5, MSN^{a}-cy5/pD and Nanosac) were added to the apical side of the Transwell insert, in which a confluent HUVEC layer was activated by TNF-α, incubated for 6 h, and quantified in the media of both apical and basolateral sides. MSN^{a}-cy5/pD and Nanosac were comparable in crossing the activated HUVEC layer (MSN^{a}-cy5/pD: 16.6±2.4% vs. Nanosac: 18.3±2.0%, FIG. 28). Interestingly, the extents of transendothelial transport of these two NPs were greater than that of MSN^{a}-cy5 (7.9±0.8%), suggesting that the albumin bound on the pD surface may contribute to the transport of MSN^{a}-cy5/pD and Nanosac across the HUVEC layer.

Nanosac Extravasates and Penetrates into Tumors Better than Hard Counterpart.

We examined the real-time intratumoral delivery of MSN^{a}-cy5/pD and Nanosac via intravital confocal microscopy using CT26 tumor-bearing mice with a dorsal window chamber. When tumors grew to 30 mm³, FITC-dextran (2000 kDa) was injected by intravenous (IV) injection to locate tumor vessels, followed by an IV injection of MSN^{a}-cy5/pD and Nanosac. The Nanosac was initially seen within vessels and then gradually extravasated and dissipated into the extravascular regions in 1 h (FIG. 4b). In contrast, MSN^{a}-cy5/pD continued to be retained in the vessel gradually losing the intensity (i.e., cleared from the system) over 1 h with little extravasation.

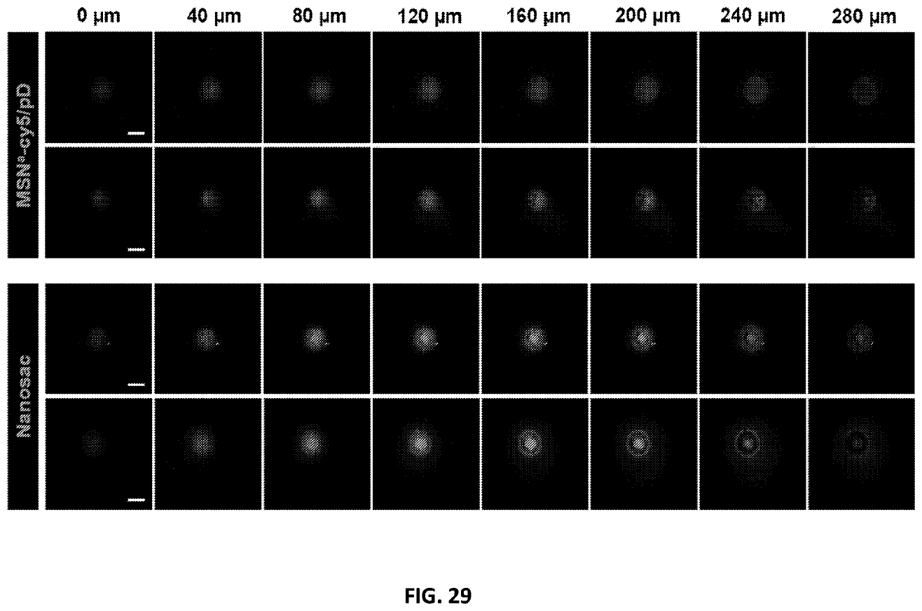

Intravital microscopy also shows that Nanosac traveled further into tumors than MSN^{a}-cy5/pD. To compare the ability of MSN^{a}-cy5/pD and Nanosac to travel in tumors quantitatively, we incubated the NPs (1 mg/mL) with CT26 tumor spheroids, 500 μm in diameter, for 4 h. Nanosac showed greater tumor penetration and accumulation than MSN^{a}-cy5/pD (FIG. 4c, FIG. 29). The mean fluorescence intensity (MFI) of Nanosac in the spheroid was 2-fold higher than that of MSN^{a}-cy5/pD (FIG. 4d). Of note, both NPs showed the peak MFI at 160 μm and decreasing MFI thereafter, which is likely due to the attenuation of fluorescence signal with the increasing depth. When compared at the same penetration depth (160 μm), the spheroid incubated with Nanosac showed stronger MFI inside than MSN^{a}-cy5/pD (FIG. 4e), indicating that the soft Nanosac penetrated into the spheroid better than the hard counterpart.

siPD-L1-Loaded Nanosac Attenuates CT26 Tumor Growth Via Immune Checkpoint Blockade.

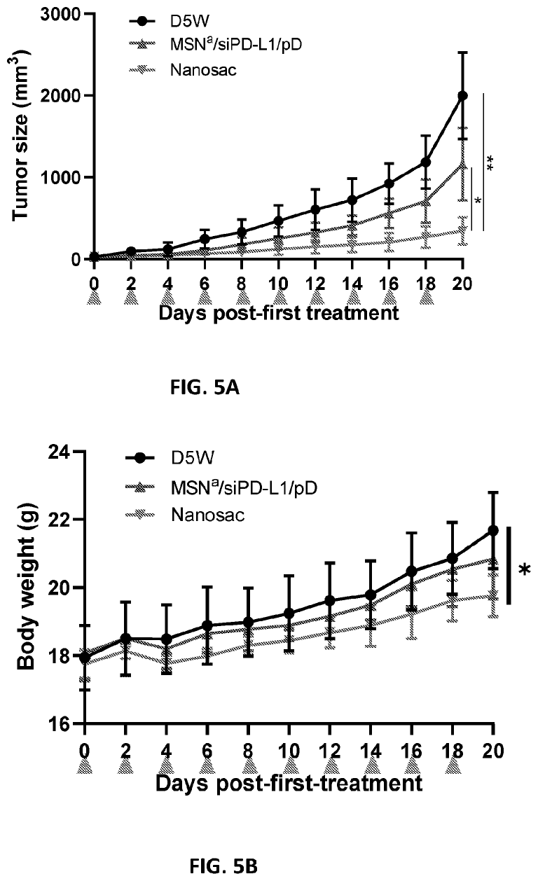

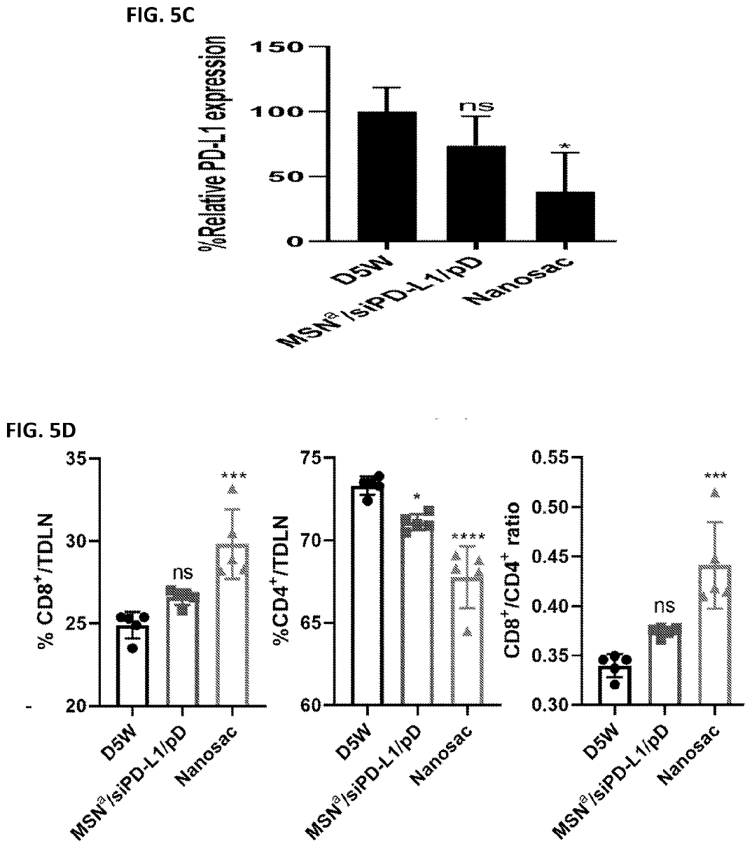

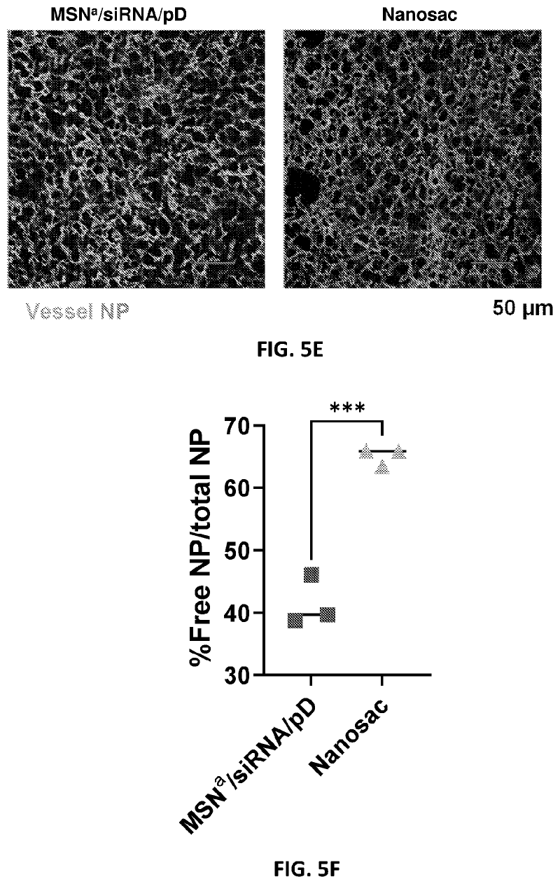

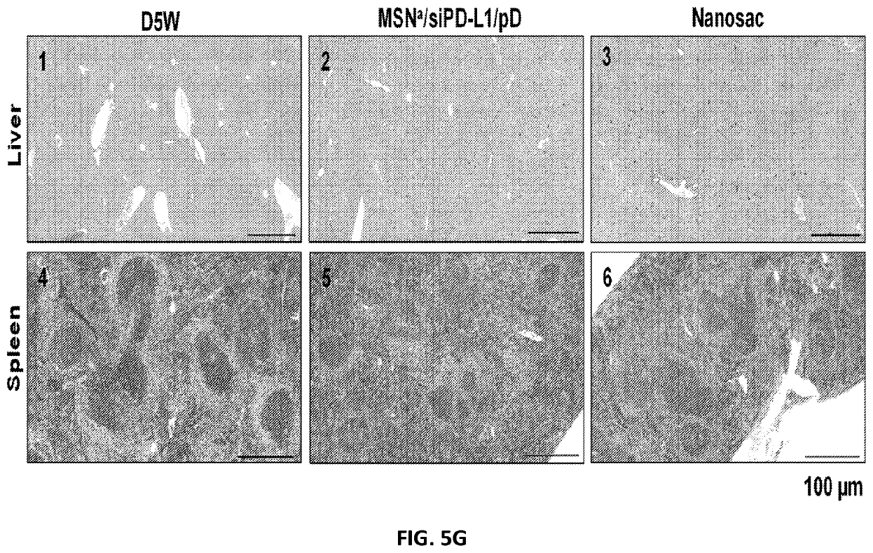

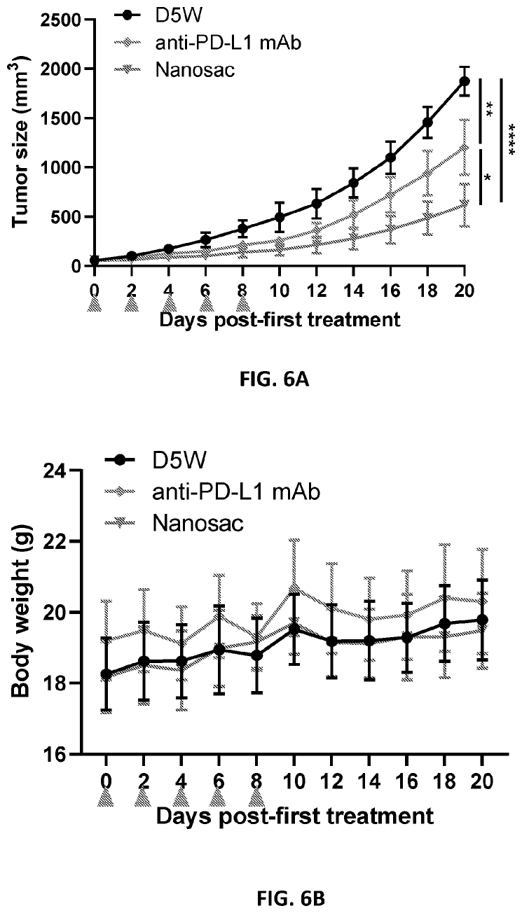

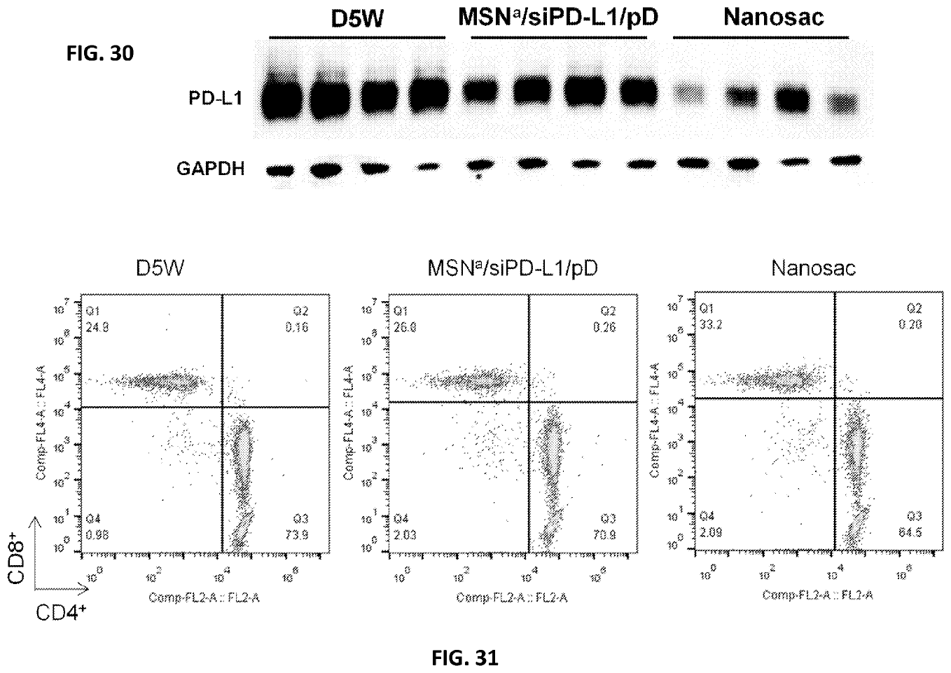

We used Nanosac to deliver siPD-L1 in CT26 colon tumor-bearing Balb/c mice for inhibiting PD-1/PD-L1 immune checkpoint interaction in tumors. The siPD-L1-loaded Nanosac or MSN^{a}/siPD-L1/pD were administered IV at a dose equivalent to siPD-L1 0.75 mg/kg/time 10 times every 2 days (q2d×10) via tail vein injection. The Nanosac-treated group showed a significant attenuation in tumor growth as compared with the 5% dextrose (D5W)-treated group (p<0.0001) and MSN^{a}/siPD-L1/pD-treated group (p<0.05) (FIG. 5a) with no weight loss (FIG. 5b). The PD-L1 expression in CT26 tumor of the Nanosac-treated group was significantly less than those of D5W- and MSN^{a}/siPD-L1/pD-treated groups (FIG. 5c, FIG. 30). To check if the silencing effect of siPD-L1 induced an immune checkpoint blockade, we examined the T-cell populations in tumor-draining lymph nodes (TDLNs). The Nanosac-treated group had more CD8⁺ T-cells and fewer CD4⁺ T-cells in TDLNs than the D5W-treated group (FIG. 5d, FIG. 31). MSN^{a}/siPD-L1/pD showed no effect on CD8⁺ T cell population and a slight reduction in CD4⁺ T cells compared to the D5W treatment. Consequently, the CD8⁺/CD4⁺ ratio in TDLNs of the Nanosac group was significantly higher than those of the D5W- and MSN^{a}/siPD-L1/pD-treated groups (FIG. 5d). This result indicates that siPD-L1-loaded Nanosac enhanced tumor infiltration of CD8⁺ T cells, while MSN^{a}/siPD-L1/pD with an equivalent dose of siPD-L1 was not as effective. The antitumor effect study was repeated with a higher unit dose of siPD-L1 (1.5 mg/kg/time, q2dx7). In this case, the growth of CT26 tumors was significantly delayed by both Nanosac and MSN^{a}/siPD-L1/pD compared to the D5W control, with the corresponding reduction in PD-L1 expression in CT26 tumor (FIG. 31). MSN^{a}/siCont/pD-treated group showed no significant difference from the D5W-treated group in both tumor growth and PD-L1 expression, indicating that the effect of Nanosac or MSN^{a}/siPD-L1/pD was not attributable to the carriers but to siPD-L1. In both regimens, none of the animals experienced significant weight loss throughout the study period (FIG. 5b, FIG. 31). Finally, we compared the anti-tumor efficacy of Nanosac (1.5 mg/kg/time, q2dx5) with the clinically approved checkpoint blockade agent, anti-PD-L1 antibody, administered in a typical preclinical regimen (intraperitoneal injection at a dose of 10 mg/kg/time, q2dx5)^{61,62} in another set of CT26 tumor-bearing Balb/c mice. The Nanosac-treated group attenuated tumor growth significantly as compared to the D5W-treated group and anti-PD-L1 antibody-treated group with no weight loss (FIG. 6).

Softness Benefits Tissue-Level Distribution of NPs.

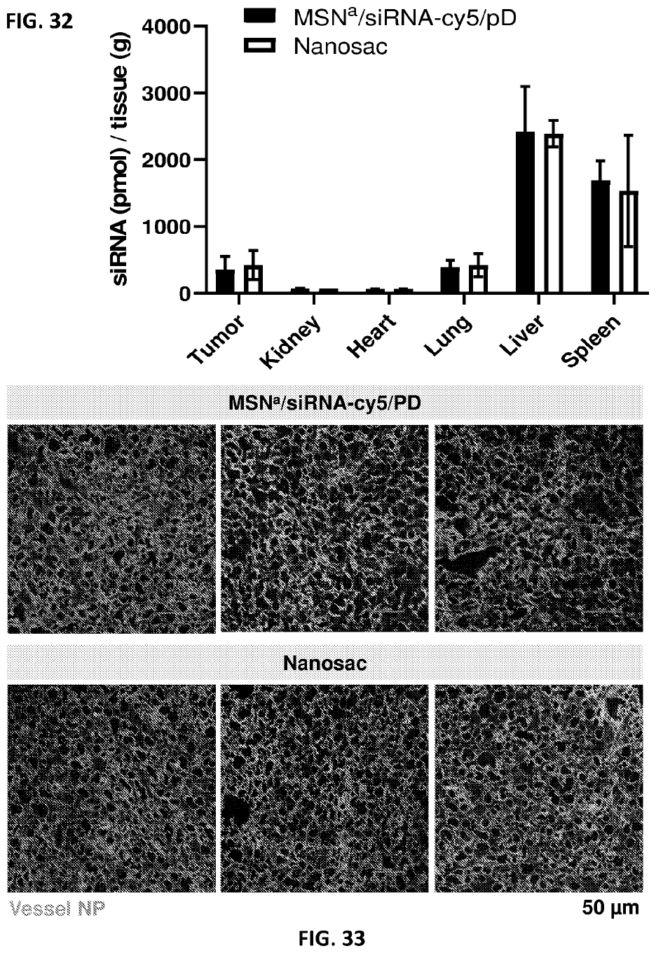

To understand the mechanism of superior antitumor effect of Nanosac, biodistribution of Nanosac and MSN^{a}/siRNA/pD was examined in Balb/c mice with CT26 tumors using siRNA-cy5 (siRNA 0.75 mg/kg). When examined 24 h after injection, the two NPs showed no significant difference in siRNA distribution in major organs (FIG. 32). The lack of difference in liver distribution between the two NPs is intriguing, given the significant difference in macrophage uptake in vitro (FIG. 4a). This may be partly explained by that the dose used in this study corresponds to less than one trillion particles, identified to be the threshold to overwhelm the Kupffer cells⁶³; thus, neither NPs may have reached a critical point to reflect their difference in macrophage uptake on the biodistribution in the liver. Likewise, tumor distribution of the two NPs was comparable. Therefore, we repeated the same experiment in another set of animals to observe tissue-level NP distribution in tumors 24 h, locating siRNA-cy5 signals (red) relative to FITC-lectin-stained blood vessels (green). The fraction of Nanosac departing from the vessels (i.e., entering tumor interstitium), quantified as free NP (red)/total NP (red+yellow), was greater than that of MSN^{a}/siRNA/pD (FIG. 5e, 5f, FIG. 33), which is consistent with intravital microscopy (FIG. 4b). These results suggest that the softness of Nanosac benefit the NP distribution on the tissue level rather than the organ level at the dose tested in this study.

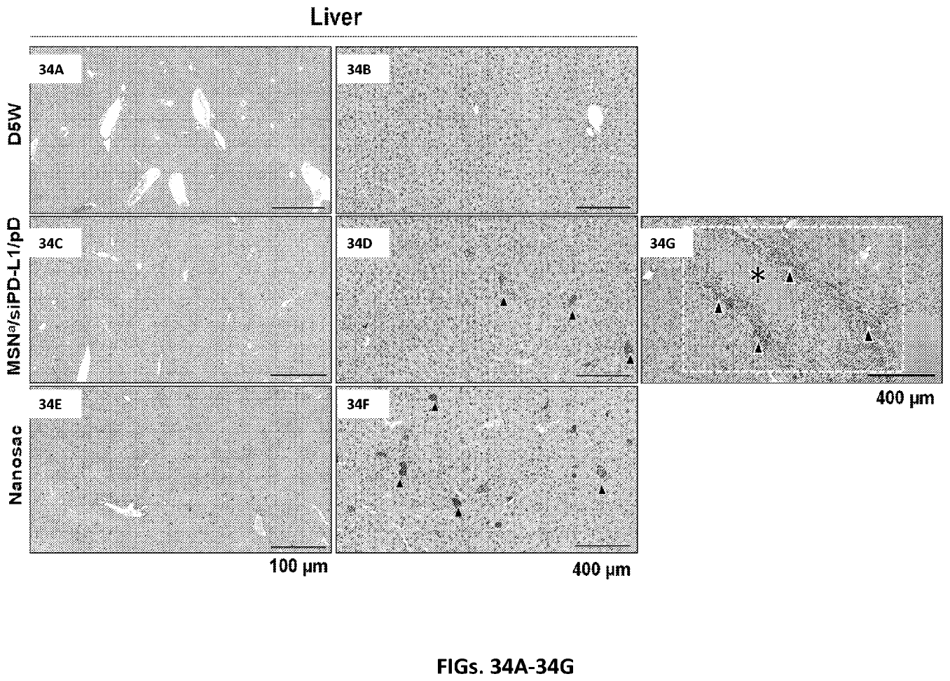

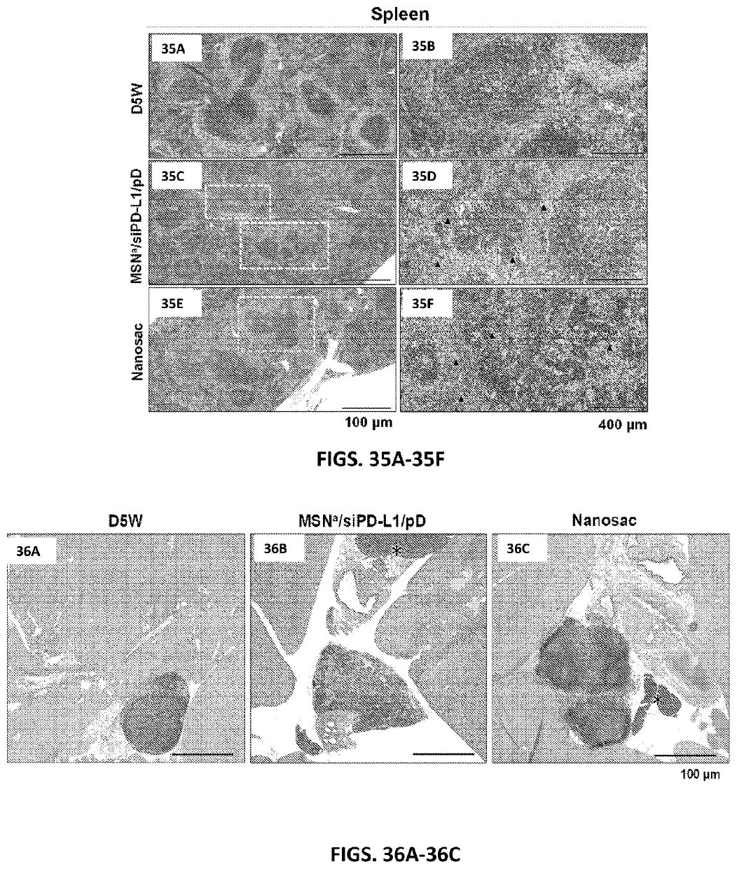

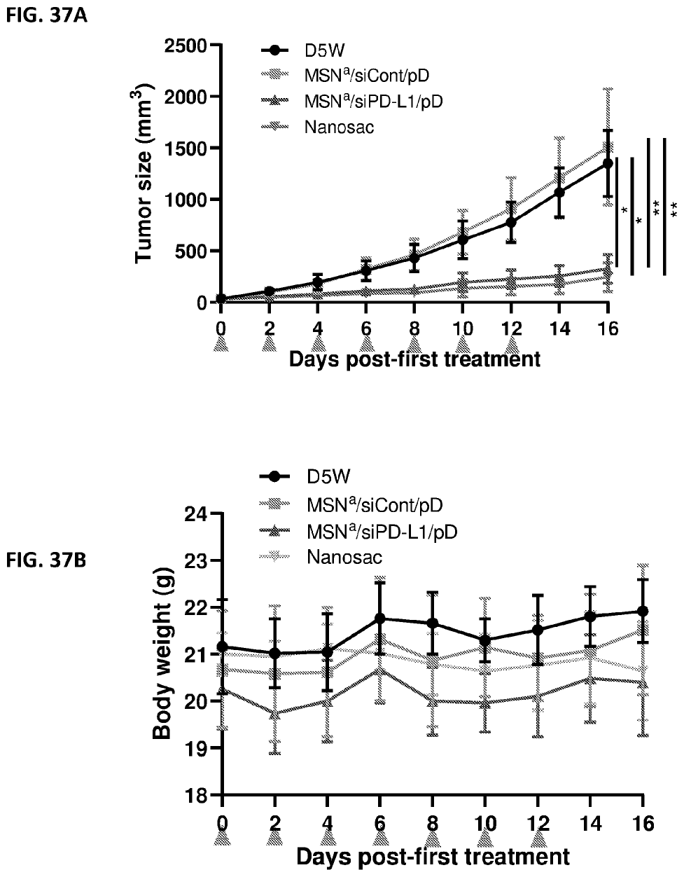

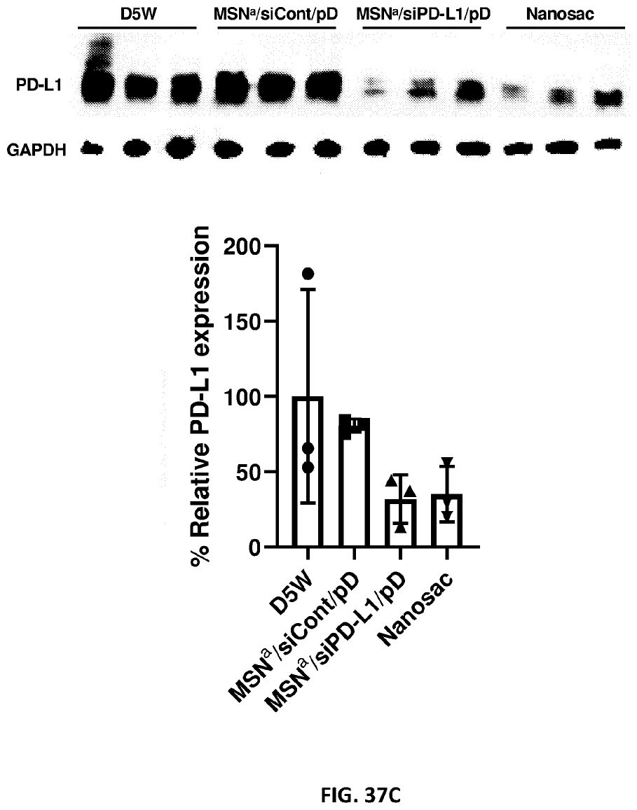

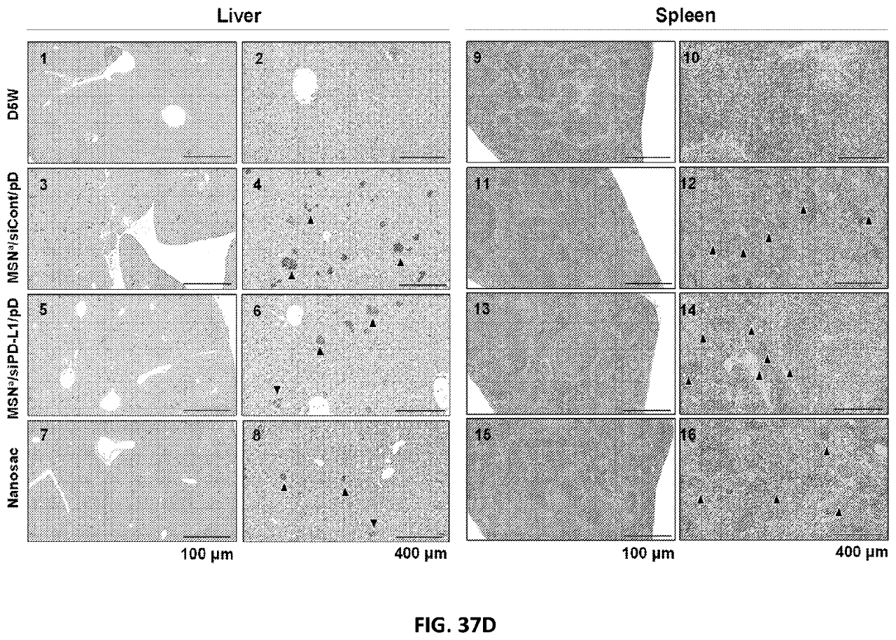

Since the biodistribution study indicated that NPs accumulated mainly in the liver and spleen (FIG. 32), we examined representative tissue samples from liver and spleen in animals treated in anti-tumor efficacy studies (FIG. 5a, 5b, FIG. 37a, 37b) to assess the effects of repeatedly administered Nanosac and MSN^{a}/siPD-L1/pD. None of the animals showed significant lesions in either organ microscopically examined (FIG. 5g). Nevertheless, MSN^{a}/siPD-L1/pD and Nanosac induced a subtle difference in tissue architecture that reflected the effect of softness on the NP transport. In the liver sections, MSN^{a}/siPD-L1/pD (FIG. 5g-2) and Nanosac (FIG. 5g-3) accumulated as multifocal randomly distributed brown pigment clusters, which were found to be NPs laden in Kupffer cells in high magnification photomicrographs (arrowheads, FIG. 34). In most animals, the NP-laden macrophages in the liver were accompanied by mild to moderate numbers of neutrophils and lymphocytes with no associated hepatocellular lesions. However, in one of the MSN^{a}/siPD-L1/pD treated animals, a portal region of liver demonstrated a severe lesion composed of abundant clusters of macrophages loaded with NPs admixed with neutrophils and other inflammatory cells with hepatocellular dropout and necrosis. The spleens of animals receiving MSN^{a}/siPD-L1/pD and Nanosac (FIG. 5e-5, e-6, FIG. 35c-f) had a less prominent zonal pattern within the white pulp (white boxes, FIG. 29c,e) due to the expansion of the sinusoidal spaces by the macrophages filled with intracytoplasmic NPs (arrowheads, FIG. 35d,f) and occasionally mildly-increased apoptosis of lymphocytes. Interestingly, the MSN^{a}/siPD-L1/pD treated animals had marked infiltration of macrophages filled with the NPs in perihepatic lymph nodes, whereas the Nanosac-treated animals showed no signs of NP accumulation (FIG. 36). This observation suggests that, even though Nanosac did not completely evade the macrophage uptake in the liver and spleen, it may have had greater agility in passing through these organs, consistent with the literature (64, 65). The contrast between the two NPs in liver or spleen distribution is even clearer in the animals treated with a higher dose. Here, MSN^{a}/siRNA/pD (MSN^{a}/siCont/pD and MSN^{a}/siPD-L1/pD) demonstrated noticeable intrahepatic accumulation within large clumps of macrophages (arrowheads, FIG. 37d-4, 37d-6), and less densely and more dispersed in Nanosac-treated livers (arrowheads, FIG. 37d-8). Consistently, NP-laden macrophages were often present in the spleen of the MSN^{a}/siRNA/pD groups but sparsely shown in the Nanosac-treated animals (arrowheads, FIG. 37d-12, 37d-14, 37d-16). The histological evaluation suggests that, even though the initial organ-level distribution appears similar between MSN^{a}/siPD-L1/pD and Nanosac, the cumulative effect of multiple injection is more favorable with Nanosac, due to the advantages in tissue level and cell level distribution of soft particles.

Discussion

Nanosac, a flexible polydopamine nanocapsule (FIG. 1, Table 1), was developed for systemic delivery of siRNA to solid tumors. Nanosac protected siRNA from nuclease challenge (FIG. 2), entered tumor cells via caveolae-mediated endocytosis, trafficked to the cytosol, and silenced target genes (FIGS. 2 and 3). Nanosac showed lower macrophage uptake and more efficient extravasation and intratumoral penetration than the hard counterpart (MSN^{a}/siPD-L1/pD) (FIG. 4). siPD-L1-loaded Nanosac, administered by intravenous injection, attenuated the growth of CT26 tumor with the evidence of immune checkpoint blockade (FIGS. 5 and 6).

TABLE 1
Description of nanoparticles (NPs)

| NP | Description |
|---|---|
| MSN | Mesoporous silica nanoparticles |
| MSN^{a} | MSN conjugated with APTES |
| MSN^{a}-cy5 | Cy5-labeled MSN^{a} |
| MSN^{a}/siRNA | MSN^{a} with siRNA loaded on the surface |
| MSN^{a}/pD | MSN^{a} covered with pD |
| MSN^{a}-cy5/pD | MSN^{a}-cy5 covered with pD |
| MSN^{a}/siRNA/pD | MSN^{a}/siRNA covered with pD |
| O/pD* | Nanocapsules after MSN core removal |
| O-cy5/pD* | Cy5-labeled nanocapsules after MSN core removal |
| O/siRNA/pD* | siRNA-loaded nanocapsules after MSN core removal |

*Collectively called Nanosac.

Here, Nanosac offered two unique features, non-cationic surface and softness, which are particularly beneficial for systemic delivery of siRNA to tumors. Nanosac did not carry positive charges to load nucleic acids, thus avoiding toxicity (66) and non-specific protein adsorption leading to NP aggregation and capillary entrapment (67). This enabled intravenous administration of siRNA-loaded Nanosac in a dose sufficient to achieve a therapeutic response. Softness enhanced transvascular and interstitial delivery of Nanosac to tumors, consistent with earlier studies (37, 39, 68). Given that Nanosac showed no difference from the hard counterpart in the endothelial interaction (FIG. 27) and transendothelial movement (FIG. 28) in a static in vitro condition, we speculate that Nanosac may have exploited transient vascular openings occurring in vivo (69) in extravasation at tumors, although it is unclear how softness may have benefited that process. The improved intratumoral penetration of Nanosac may be explained by the deformability in the tumor interstitium. Supporting this possibility, a study with polymeric NPs of varying rigidities has shown that soft NPs translocate a porous membrane and penetrate into collagen gel more easily than hard NPs (38). Softness also helped reduce macrophage uptake of Nanosac (FIG. 4a). A recent report of silica nanocapsules with controlled softness attributes the reduced phagocytosis of soft particles to their deformation induced by actin-rich cell protrusions, which slows down the particle uptake (70). For these reasons, softness has been one of the motivations to pursue cell-derived particles such as extracellular vesicles as a drug carrier (39). While sharing the advantage of soft NPs, Nanosac avoids challenges related to cell-derived vesicles, such as scalability, heterogeneity, control of payload loading, immunogenicity, and potential risk of disease transfer (71).

The pD surface of Nanosac has brought at least three additional benefits toward siRNA delivery to tumors based on its interaction with serum albumin (FIG. 3c, d). First, it allows Nanosac to interact with cells in distinct mechanisms. While Nanosac showed lower macrophage uptake than the hard counterpart in vitro, its uptake by CT26 cells and endothelial cells was unaffected by the softness (FIG. 27). We speculate that these cells may have interacted with Nanosac via the albumin on the pD surface. We previously reported that pD-coated polymeric NPs selectively bound to albumin preserving its native conformation (41). With the albumin-bound (albuminylated) surface, the pD-coated polymeric NPs took advantage of albumin-mediated interactions with endothelial cells and tumor cells, to achieve greater drug delivery to tumors than control NPs that had non-specific, denaturing interactions with serum proteins (41). The surface-bound albumin may have exploited increasing demand of cancer cells for albumin as a source of energy and nutrients (72, 73) to enhance cancer cell uptake of NPs and interacted with glycoproteins expressed on peritumoral endothelium (74-77). Nanosac, with the pD surface, was albuminylated in serum (FIG. 3d); therefore, it may have benefited from the surface-bound albumin in the interaction with tumor cells and endothelial cells in the same manner as the polymeric NPs (41).

The pD coated NPs (MSN^{a}-cy5/pD and Nanosac) were transported across the endothelial layer better than MSN^{a}-cy5 with no pD coating (FIG. 28). This suggests that albuminylation may also have contributed to the extravasation of Nanosac via the albumin-mediated interaction with endothelial cells, leveraging the effect of softness. In the same vein, a recent study with gold NPs has suggested that, in the absence of the enhanced permeability and retention (EPR) effect, an active transendothelial pathway, possibly the caveolae-mediated transcytosis via the albumin corona, may account for the NP extravasation into tumors (78). On the other hand, it is worth reminding that albumin has long been used as a dysopsonin to reduce the binding of other proteins on NPs that lead to the RES uptake (79-82), thereby extending the circulation time of NPs (79, 80). The multiple roles of albumin suggest that the surface-bound albumin may enhance both tumor accumulation and cellular interaction of NPs. This distinguishes the in-situ adoption of albumin from traditional PEGylation, which has shown limitations in pursuing both (23).

Moreover, the albuminylated Nanosac took caveolae-mediated endocytosis to enter CT26 cells (FIG. 3a), consistent with the uptake pathway of albumin (83) and albumin-bound drugs (84, 85). The uptake pathway has an important implication in the intracellular delivery of siRNA. While the ultimate fate of the internalized caveolae remains controversial (86) (fuse with endosomes and follow the degradative pathway (87) or take alternative cellular compartments to avoid degradative pathway like viruses do (88)), our microscopic observation indicates that Nanosac avoids the sequestration in acidic lysosomal compartments and traffics to the cytosol (FIG. 3f), where it unloads siRNA likely by intracellular ROS (FIG. 3i) to achieve efficient gene silencing (FIG. 2). This unique intracellular trafficking pathway allows Nanosac-encapsulated siRNA to avoid lysosomal degradation, a significant advantage for siRNA delivery. Of note, the uptake and trafficking of NPs appear to be influenced by the quality and purity than the quantity of the surface-bound albumin. As shown in SDS-PAGE and proteomics analysis of the protein corona, MSN^{a} captured more albumin than MSN^{a}/pD or Nanosac (FIGS. 3c, 3d) but trafficked predominantly to lysosomes (FIG. 3f), which may be attributable to the additional proteins interfering with albumin (FIG. 3d) and the denaturation of the surface-bound albumin (FIG. 3e). This observation underscores the advantage of pD as a non-denaturing surface for selective albumin adsorption.

### CONCLUSION

We have developed Nanosac, soft non-cationic nanocapsules, for systemic delivery of siRNA. Nanosac is produced by sequential attachment of siRNA and polydopamine on a sacrificial MSN core, followed by removal of the MSN. Encapsulating siRNA in the capsules, Nanosac avoids the issues common to cationic gene carriers, such as toxicity and non-specific protein binding while protecting siRNA from RNase. Nanosac entered tumor cells by caveolae-mediated endocytosis, likely via albumin recruited from serum, trafficked to the cytosol, and silenced target genes. Due to the softness, Nanosac showed lower macrophage uptake, greater extravasation and penetration into tumors better than the hard counterpart. As a carrier of siPD-L1, Nanosac facilitated CD8⁺ T cell recruitment to tumors and controlled tumor growth significantly better than the hard counterpart. These results support that Nanosac offers enabling features for systemic siRNA delivery to tumors.

Materials and Methods

Materials

All chemicals including tetraethyl orthosilicate (TEOS), cetyltrimethylammonium chloride (CTAC), triethanolamine, 3-aminopropyl)triethoxysilane (APTES), ammonium hydrogen difluoride, ammonium fluoride, 3-(4,5-dimethyl-2-thiazolyl)-2,5-diphenyl-2H-tetrazolium bromide (MTT), chlorpromazine, methyl-β-cyclodextrin, and amiloride hydrochloride were purchased from Sigma Aldrich (St. Louis, MO, USA), unless specified otherwise. Dopamine hydrochloride was purchased from Alfa Aesar (Ward Hill, MA, USA). Cy3-labeled GAPDH siRNA, luciferase siRNA, RNase A, wheat germ agglutinin-488, Hoechst 33342, Lysotracker green, and Lipofectamine 2000 were purchased from Invitrogen (Eugene, OR, USA). PD-L1 siRNA (sense, 5′-CCCACAUAAAAAACAGUUGTT-3′ (SEQ ID NO: 1); antisense, 5′-CAACUGUUUUUUAUGUGGGTT-3′ (SEQ ID NO: 2)); and negative control siRNA (sense, 5′-UGAAGUUGCACUUGAAGUCdTdT-3′ (SEQ ID NO: 3); antisense, 5′-GACUUCAAGUGCAACUUCAdTdT-3′ (SEQ ID NO: 4)) were purchased from Integrated DNA Technologies (IDT, Coralville, Iowa, USA). Sulfo-cyanine5 NHS ester was purchase from Lumiprobe (Hunt valley, MD, USA). Luciferase assay kit was purchased from Promega (San Luis Obispo, CA, USA). GAPDH ELISA kit was purchased from Abcam (Burlingame, CA, USA). PD-L1 ELISA kit was purchased from Biomatik (Wilmington, DE, USA). Gibco Dulbecco's Modified Eagle's medium (DMEM) and Gibco RPMI 1640 medium (RPMI) were purchased from ThermoFisher Scientific (Waltham, MA, USA). Vascular cell basal medium and endothelial cell growth kit-BBE were purchased from American Type Culture Collection (ATCC, Manassas, VA, USA). PE anti-mouse CD4, FITC anti-mouse CD3, and APC anti-mouse CD8a antibodies were purchased from BioLegend (San Diego, CA, USA). Anti-PD-L1 antibody (clone 10F.9G2) was purchased from Bio X Cell (Lebanon, NH, USA). FITC-Lectin was purchased from Vector Laboratories (Burlingame, CA).

Preparation of siRNA-Loaded Nanocapsules

siRNA-loaded nanocapsules (O/siRNA/pD, Nanosac) were prepared by adsorbing siRNA on amine-modified mesoporous silica nanoparticles (MSNs), coating the siRNA-bound MSNs with pD, and removing the sacrificial MSNs. First, MSNs were synthesized according to the sol-gel procedure (42) with slight modification. CTAC (25%, 5 mL), a cationic surfactant, was added to 15 mL of deionized water, stirred at 300 rpm for 15 min at 75° C. to form micelles serving as mesopore templates, and mixed with 0.8 mL of 10% triethanolamine at 75° C. for additional 15 min. To the CTAC/triethanolamine micelles, TEOS (1.5 mL) was added at a rate of ˜30 drops per minute and stirred for 1 h at 300 rpm at 80° C. to form silica layers around the micelle clusters. CTAC was then removed by refluxing the mixture with methanol and HCl (500:19, v/v) at room temperature for 24 h. The MSNs was centrifuged at 20,000 rcf for 20 min and washed three times with methanol. MSNs (50 mg/mL) were mixed with 25 μL of APTES in ethanol at room temperature for 24 h to modify the surface with amine groups. Thereby formed MSN-APTES (MSN^{a}) particles were centrifuged at 20,000 rcf for 20 min and washed three times with ethanol. The purified MSN-APTES were mixed with siRNA in HEPES-buffered saline (pH 7) at a weight ratio of 50/1 and incubated for 5 min. The siRNA-loaded MSNs (MSN^{a}/siRNA) were coated with polydopamine (pD) layer by incubation in 1 mL of 1 mg/mL dopamine hydrochloride solution in Tris buffer (10 mM, pH 8.5) for 6 h at room temperature with rotation. Finally, the pD-coated, siRNA-loaded MSNs (MSN^{a}/siRNA/pD) were dispersed in 50 μL of deionized water and added to 200 μL of buffered oxide etch solution (2M HF/8M NH₄F, pH 5) to remove the sacrificial MSNs. After 5 min, the resulting nanocapsules (O/siRNA/pD, Nanosac) were washed three times by centrifugation (4600 rpm for 5 min). For fluorescent labeling of MSNs, 25 mg of MSN^{a} was dispersed in anhydrous dimethylformamide (8 mL) containing sulfo-cy5-NHS (1 mg) and triethylamine (80 μL). The reaction solution was stirred in dark for 24 h. The labeled MSN^{a} (MSN^{a}-cy5) were washed with ethanol five times and dispersed in deionized water. To test the feasibility of lyophilization, Nanosac was lyophilized by the Labconco FreeZone 4.5 Liter −84 C Benchtop Freeze Dryer (Kansas City, MO, USA) with a varying amount of trehalose as a lyoprotectant. The dried Nanosac was reconstituted in deionized water and analyzed by the Malvern Zetasizer Nano ZS90 (Worcestershire, United Kingdom) and gel electrophoresis.

Characterization of NPs

Intermediate NPs (MSN, MSN^{a}, MSN^{a}/siRNA, and MSN^{a}/siRNA/pD) and siRNA-loaded nanocapsules (O/siRNA/pD, Nanosac), which were collectively called ‘NPs,’ were dispersed in phosphate buffer (10 mM, pH 7.4), and their sizes and zeta potentials were determined by the Malvern Zetasizer Nano ZS90 (Worcestershire, United Kingdom). Their morphology was examined by a FEI Tecnai T20 transmission electron microscope (Hillsboro, OR) after negative staining with 1% uranyl acetate or 1% phosphotungstic acid. For AFM analysis, samples were prepared by placing a droplet of NP suspension on a 300-mesh copper grid (Electron Microscopy Sciences, Hatfield, PA, USA). Excess samples were removed by blotting paper and the grid was air-dried prior to measurement. The images and Young's moduli of the NPs were obtained by an Asylum Cypher (Oxford instruments, Abingdon, United Kingdom). The Young's modulus of NPs was determined by fitting the force-distance curve by the Hertz equation (1) (47, 48).

$$F = \frac{E}{1 - {v}^{2}} \frac{tan β}{\sqrt{2}} {δ}^{2} \qquad ( 1 )$$

Gel Retardation Assay for Testing siRNA-Loading Capacity and siRNA Stability

siRNA-loading capacity of MSN^{a} was evaluated by the agarose gel retardation assay. siRNA-loaded MSNs (MSN^{a}/siRNA) complexes were prepared varying the MSN^{a}/siRNA weight ratio from 1/1 to 50/1. The complexes were loaded in 2% agarose gel and run in 0.5×TAE buffer at 80 V for 40 min. The gel was stained with ethidium bromide, and siRNA bands were detected at 302 nm using Azure C300 (Dublin, CA, USA). For stability testing, siRNA or NPs were challenged with 166 U/mL RNase for 15 min±8 mg/mL SDS for additional 2.5 h or 50% FBS for 1 h, both at 37° C., and analyzed by agarose gel electrophoresis. To quantify siRNA loaded in Nanosac (O/siRNA/pD), the NPs were dispersed in dilute HCl solution (pH 3) and incubated for 72 h. The samples were centrifuged at 16,000 rcf for 10 min, and the supernatant and pellet were separately analyzed by agarose gel electrophoresis.

Cell Culture

CT26 mouse colon carcinoma (ATCC), luciferase-expressing 4T1 mouse mammary carcinoma cells (4T1-luc, donation of Prof. Michael Wendt at Purdue University), and J774A.1 macrophages (ATCC) were cultured in DMEM medium, complemented with 10% fetal bovine serum (FBS), 100 U/mL penicillin and 100 μg/mL streptomycin. Human umbilical vein endothelial cells (HUVEC, ATCC) were cultured in vascular cell basal medium with endothelial cell growth kit-BBE (ATCC).

Cytotoxicity of NPs

CT26 cells were seeded in a 96 well plate at a density of 10,000 cells per well and cultured at 37° C. in 5% CO₂. After overnight incubation, the culture medium was replaced with fresh medium containing MSN^{a}, MSN^{a}/pD, and Nanosac at 5-500 μg/ml and incubated for 48 h. Cell proliferation was quantified by the MTT (3-(4,5-dimethylthiazol-2-yl)-2,5-diphenyltetrazolium bromide) assay, where the cells were treated with 75 μg of MTT and incubated for 4 h. The formazan crystals were dissolved in DMSO and quantified by a SpectraMax M3 microplate reader (Molecular Devices, CA, USA) at the wavelength of 562 nm. The cell viability was defined as the absorbance divided by that of control cells that did not receive any treatment.

Confocal Microscopy

Cells were seeded on a cover glass placed in a 12 well plate and incubated for 24 h. The medium was replaced with a suspension of Cy3 or Cy5-labeled NPs (MSN, MSN^{a}/pD, and Nanosac) and incubated for a specified time period at 37° C. The cells were then gently rinsed with PBS twice and fixed with 4% paraformaldehyde in PBS for 15 min. Cell membrane was stained with FITC-labeled wheat germ agglutinin (5 μg/ml) for 10 min. Hoechst 33342 nuclear stain (2 μM) was added 5 min prior to the imaging. Confocal microscopy was performed by the Nikon A1R confocal microscope (Melville, NT, USA).

Gene Silencing

Three model siRNAs targeting GAPDH, luciferase, and PD-L1 expression (siGAPDH, siLuc, and siPD-L1) were used to test siRNA delivery via MSN^{a}, MSN^{a}/pD, and Nanosac. Non-specific siRNA (siCont) was used as a negative control for each evaluation. siGAPDH was tested with CT26 cells. The cells were seeded in 12-well plates at a density of 2×10⁵ cells per well, grown for 24 h, and incubated with no treatment or MSN^{a}, MSN^{a}/pD, and Nanosac loaded with siGAPDH or siCont (at a concentration equivalent to 100 nM siRNA) in complete medium for 48 h. The cells were rinsed with PBS twice and treated with 100 μL of lysis buffer for 15 min. The GAPDH level in the cell lysate was quantified by a standard GAPDH assay kit (Abcam, GAPDH ELISA Kit). siLuc was tested with 4T1-luc cells. The cells were seeded in 12-well plates at a density of 10 cells per well, grown for 24 h, and incubated with no treatment or the NPs loaded with siLuc or siCont (at 150 nM siRNA) in complete medium for 48 h. The cells were lysed in passive lysis buffer for 10 min and analyzed for luciferase activity by the Luciferase Glow Assay Kit (Promega). siPD-L1 was tested with CT26 cells. The cells were plated in 6-well plates at a density of 10⁵ cells per well, incubated for 24 h, pretreated with 100 ng/mL of IFN-γ for 12 h to induce PD-L1 expression, and incubated with no treatment or the NPs loaded with siPD-L1 or siCont (at 200 nM siRNA) in complete medium for 48 h. In another set, the IFN-γ-pretreated CT26 cells were incubated with NPs loaded with siPD-L1 at varying concentrations (50-200 nM siPD-L1). The cells were lysed with a cell lysis buffer containing 1% protease inhibitor and analyzed for PD-L1 expression by the PD-L1 ELISA kit (Biomatik).

Western Blot

The siPD-L1 treated CT26 cells were lysed with lysis buffer containing 1% protease inhibitor. The protein content in the cell lysates was quantified by the BCA assay. The lysates were boiled in Laemmli buffer for 5 min, resolved by 10% SDS-polyacrylamide gel electrophoresis, and transferred to polyvinylidene fluoride membrane. The membrane was blocked by 5% nonfat dried milk in TBST buffer (pH 7.4, 20 mM Tris, 150 mM NaCl, and 0.05% Tween 20) for 1 h at room temperature. The membrane was incubated with anti-PD-L1 and anti-GAPDH antibodies for 24 h at 4° C. The membrane was washed three times and incubated with secondary IgG-HRP antibody for 1 h at room temperature. The membrane was washed three times, and protein bands were imaged by Azure C300 (Dublin, CA).

NP Uptake by J774A.1, HUVEC, and CT26 Cells

J774A.1 cells were seeded in 6-well plates at a density of 10⁶ cells per well. HUVEC and CT26 cells were seeded in 12-well plates at a density of 10⁵ cells per well. After overnight, J774A.1 cells were treated with MSN^{a}-cy5/pD or Nanosac for 30 min or 2 h. HUVEC and CT26 cells were incubated with MSN^{a}-cy5/pD or Nanosac for 2 h or 6 h. The cells were then rinsed with PBS twice and lysed in dilute HCl (pH 3) solution with 3 cycles of freezing and thawing. Cy5 was retrieved from the cell lysate by 10 sec probe sonication at 30% amplitude, followed by 72 h incubation, and quantified by Synergy Neo2 plate reader (Biotek, Chittenden County, VT, USA).

Transendotheial Transport of NPs

HUVEC cells were seeded at a density of 80,000 cells per well in a Transwell insert (3 μm pore) pre-coated with rat-tail collagen type I. Transendothelial electrical resistance (TEER) across the HUVEC layer was monitored daily by EVOM2™ epithelial voltohmmeter (World Precision Instruments, Sarasota, FL, USA). When the TEER value reached a plateau (indicating confluency), the HUVEC layer was incubated with TNF-α (10 ng/mL) for 4 h. After rinsing the cell layer twice with PBS to remove TNF-α, 0.1 mg of MSN^{a}-cy5, MSN^{a}-cy5/pD, or Nanosac were added to the apical side of the Transwell and incubated for 6 h. The media in apical and basolateral sides were collected, and the fluorescence intensity of the collected media were measured by Synergy Neo2 plate reader (Biotek, Chittenden County, VT, USA) to quantify the NPs in each side.

Mechanism of NP Uptake

First, inhibitors of endocytosis pathways (chlorpromazine, methyl-β-cyclodextrin, and amiloride hydrochloride) were tested on CT26 cells to identify safe concentration ranges. CT26 cells were seeded at a density of 10⁴ cells per well in a 96 well plate and incubated for overnight. At 70-80% confluency, the cells were incubated with different endocytosis inhibitors for 30 min and then rinsed three times with PBS. The cell viability was determined by the MTT assay. Next, CT26 cells were seeded at a density of 10⁵ cells in a 12 well plate, incubated to 70-80% confluency, treated with chlorpromazine (5 nM), methyl-β-cyclodextrin (5 mM), or amiloride hydrochloride (1 mM) for 30 min, and rinsed with PBS three times. The cells pre-treated with each inhibitor were incubated with MSN^{a}-cy5, MSN^{a}-cy5/pD, or Nanosac (0.5 mg/ml) for 6 h. Cy5 levels in the cells were quantified as described in the NP uptake section. To confirm the cellular uptake mechanism, the NPs and lysosomes were located by confocal microscopy. CT26 cells were incubated with MSN^{a}-cy5, MSN^{a}-cy5/pD, and Nanosac for 6 h. Lysosomes were labeled with LysoTracker Green (200 nM) for 30 min, and the nuclei were stained with Hoechst 33342 (2 μM) for 5 min. NPs, LysoTracker, and Hoechst were detected at X/km of 350 nm/461 nm, 488 nm/520 nm, 646 nm/662 nm, respectively.

siRNA Release

Nanosac equivalent to 10 μM of siPD-L1 was suspended in 100 μL of deionized water with pH 5.2, pH 6.2, pH 7.4, or H₂O₂ (100 μM). The Nanosac suspensions were incubated at 37° C. under constant agitation. Nanosac suspension was sampled at predetermined time points, loaded in 2% agarose gel, and run in 0.5×TAE buffer at 80 V for 40 min. The gel was stained with ethidium bromide, and the siRNA bands were detected at 302 nm using Azure C300 (Dublin, CA, USA). The percent siPD-L1 release was calculated as (band intensity of released siPD-L1/band intensity of 10 μM of siPD-L1)×100.

Protein Corona Analysis

The composition of protein corona forming on NPs was analyzed by SDS-PAGE. Four milligrams of MSN^{a}-cy5, MSN^{a}-cy5/pD, and Nanosac were incubated in 1 mL of 50% FBS for 2 h with rotation. The NPs were washed twice with PBS, boiled in Laemmli buffer for 5 min, and resolved by 12% SDS-polyacrylamide gel electrophoresis. The gel was stained by Coomassie staining and imaged by Azure C300 (Dublin, CA). Protein bands in gel were excised and analyzed by liquid-chromatography mass-spectrometry (LC-MS/MS) as described below.

The status of albumin bound to NP surface was determined by pulse proteolysis (89). Four milligrams of MSN^{a}-cy5 and MSN^{a}-cy5/pD were incubated in 1 mL of human serum albumin (10 mg/mL) for 2 h with rotation. The NPs were centrifuged at 16000 rcf for 10 min and washed with PBS twice. The albumin-bound NPs were treated with 0.2 mg/mL of thermolysin in HEPES buffer (pH 7.4, 20 mM) containing 100 mM NaCl and 10 mM CaCl₂. After 3 min incubation at room temperature, 5 μL of 50 mM EDTA was added to a 15 μL aliquot to quench proteolysis. For the control, 0.1 mg/mL of native albumin or denatured albumin (boiled at 95° C. for 10 min) were treated in the same manner. The samples were analyzed by SDS-PAGE. The protein bands were detected with Azure C300 to analyze the extent of proteolysis of surface-bound albumin. The percent digestion was calculated as (1−band intensity of albumin after proteolysis/band intensity of albumin prior to proteolysis)×100.

LC-MS/MS Analysis of Protein Corona

In-gel protein digestion: Gel bands were excised and de-stained 3 times with 25 mM ammonium bicarbonate (ABC)/50% acetonitrile (ACN) and once with 80% ACN. The gel pieces were dried in a vacuum centrifuge for 15 min. Reduction of disulfide bond was carried out using 10 mM dithiothreitol in 25 mM ABC at 55° C. for 1 h. Alkylation of cysteine was carried out using 55 mM iodoacetamide in 25 mM ABC at room temperature in the dark for 45 min. Gels were then washed twice with 25 mM ABC/50% ACN, dried and transferred to Barocycler tubes and digested in for 2 h in Barocycler at 50° C. and 20,000 psi (50 seconds at 20,000 psi, 10 seconds at atmospheric pressure for a total of 120 cycles or 2 h) using LysC/trypsin protease mix (Promega) at an enzyme-to-substrate ratio of 1:25. After digestion, supernatants were transferred to a new tube and remaining peptides were extracted using 60% ACN/5% trifluoroacetic acid (TFA). Pooled peptides were dried in a vacuum centrifuge to prepare for LC-MS/MS analysis.

Mass Spectrometry analysis: Samples were analyzed in the Dionex UltiMate 3000 RSLC nano System combined with the Q-Exactive High-Field (HF) Hybrid Quadrupole Orbitrap MS (Thermo Fisher Scientific). Peptides were re-suspended in 3% ACN/0.1% Formic Acid (FA)/96.9% MilliQ, and 5 μL was used for LC-MS/MS analysis. Peptides were separated using a trap (300 μm ID×5 mm packed with 5 μm 100 Å PepMap C18 medium) and the analytical columns (75 μm×50 cm packed with 2 μm of 100 Å PepMap C18 medium) (Thermo Fisher Scientific) using a 120 min method at a flow rate of 300 nL/min. Mobile phase A consisted of 0.1% FA in water and mobile phase B consisted of 0.1% FA in 80% ACN. The linear gradient started at 5% B and reached 30% B in 80 min, 45% B in 91 min, and 100% B in 93 min. Next, the column was held at 100% B for the next 5 min before bringing back to 5% B and held for 20 min to equilibrate the column. The column temperature was maintained at 37° C. MS data were acquired with a Top 20 data-dependent MS/MS scan method with a maximum injection time of 100 ms, a resolution of 120,000 at 200 m/z. Fragmentation of precursor ions was performed by high-energy C-trap dissociation (HCD) with the normalized collision energy of 27 eV. MS/MS scans were acquired at a resolution of 15,000 at m/z 200. The dynamic exclusion was set at 20 s to avoid repeated scanning of identical peptides.

Bioinformatics and data analysis: The raw MS/MS data were processed using MaxQuant (v1.6.3.3) (90) with the spectra matched against the bovine (Bos Taurus) protein database downloaded from Uniprot (http://www.uniprot.org) on 5/20/2020. Data were searches using trypsin/P and LysC enzyme digestion allowing for up to 2 missed cleavages. MaxQuant search was set to 1% FDR both at the peptide and protein levels. The minimum peptide length required for database search was set to seven amino acids. Precursor mass tolerance of t 10 ppm, MS/MS fragment ions tolerance of t 20 ppm, oxidation of methionine protein N-terminal acetylation (K) were set as the variable modifications and carbamidomethylation of cysteine (C) was set as a fixed modification. The “unique plus razor peptides” were used for peptide quantitation. Razor peptides are the non-redundant, non-unique peptides assigned to the protein group with most other peptides. LFQ intensity values were used for relative protein abundance measurement. Proteins detected with at least 1 unique peptide and at least 2 MS/MS counts were only included for the final analysis.

NP Penetration into Tumor Spheroids

3×10³ of CT26 cells were seeded in a round-bottom 96 well plate (Corning), briefly centrifuged at 3,000 rcf for 5 min to aggregate at the bottom of the plate, and incubated for 72 h to form spheroids. The spheroids were treated with MSN^{a}-cy5/pD or Nanosac (1 mg/mL as NPs) for 4 h, rinsed with PBS, and fixed with 4% paraformaldehyde for 30 min. Z-stack confocal images of the spheroid were obtained with 20 μm intervals from the bottom to the middle of the spheroids, by the Nikon A1R confocal microscope (Melville, NT, USA). Cy5 was detected at λ_{Ex}/λ_{Em} of 646 nm/662 nm.

Intravital Confocal Microscopy

5-6 weeks old female Balb/c mouse were purchased from Envigo (Indianapolis, IN) and acclimatized for 7 days prior to the procedure. All animal procedures were approved by Purdue Animal Care and Use Committee, in conformity with the NIH guidelines for the care and use of laboratory animals. For the real-time observation of NP transport in tumors, a dorsal window chamber was installed in the back of a mouse (91-93). A Balb/c mouse was anesthetized by 2.5% isoflurane in oxygen flow using an anesthesia machine (Matrx VMS, Midmark). A window chamber was surgically implanted onto the dorsal skinflap, where 10⁶ of CT26 cells suspended in 25 μL of PBS were subsequently injected. The tumor-inoculated skinflap was covered with a coverslip (1 cm diameter) and monitored every other day. When the tumor size reached ˜30 mm³, the mouse was given 100 μL of wheat germ agglutinin (WGA) 488 (1 mg/mL) for blood vessel staining, followed by MSN^{a}-cy5/pD or Nanosac (6 mg per mouse) injection, via a preinstalled mouse tail vein catheter. Intravital imaging was performed under isoflurane anesthesia using the Nikon Intravital MP confocal upright microscope equipped with a 10× objective. WGA 488 and cy5 signals were detected at λ_{Ex}/λ_{Em} of 488 nm/520 nm and 646 nm/662 nm, respectively.

Systemic siRNA Delivery to Subcutaneous Tumor

Tumor-bearing mice were prepared by subcutaneous injection of 3×10⁵ CT26 cells suspended in 100 μL of growth medium in the upper flank of the right hind leg of a 5-6 weeks old female Balb/c mouse. When the tumor size reached 50 mm³, animals received intravenous injection of PBS, MSN^{a}/siCont/pD, MSN^{a}/siPD-L1/pD, or Nanosac (all equivalent to siRNA 0.75 mg/kg/time, q2dx10 or 1.5 mg/kg/time, q2dx7) via tail vein. The treatment was repeated seven times with a 2-day interval. In another set of experiment, Nanosac (equivalent to siPD-L1 1.5 mg/kg/time, q2dx5) was compared with anti-PD-L1 antibody (10 mg/kg/time, q2dx5, intraperitoneal injection). Tumor size was monitored every 2 days. The length (L) and width (W) of each tumor were measured by a digital caliper, and the volume (V) was calculated by the modified ellipsoid formula: V=(L×W²)/2 (94). To evaluate PD-L1 silencing in CT26 tumors by siPD-L1, tumors were sampled at sacrifice and analyzed by Western blotting. Tumors were homogenized by the Omni Tissue Master 125 homogenizer (Kennesaw, GA) and lysed with a lysis buffer containing 1% protease inhibitor. The tumor lysates were analyzed by Western blot as described previously. For histological evaluation, the livers and spleens were harvested and fixed in 10% neutral buffered formalin. The fixed tissues were embedded in paraffin, sectioned, and stained with hematoxylin and eosin. For immunophenotyping, tumor draining lymph nodes were harvested, cut into pieces, and filtered through 100 μm and 40 μm cell strainers. The single cell suspension was incubated with anti-mouse CD16/32 antibody to block non-specific binding and identified by anti-CD3 (FITC), CD4 (PE) and CD8 (APC) antibodies. The stained cells were analyzed by the BD Accuri C6 Flow Cytometer (BD Bioscience, Bedford, MA).

Biodistribution

Tumor-bearing mice were prepared by subcutaneous injection of 3×10⁵ CT26 cells suspended in 100 μL of growth medium in the upper flank of the right hind leg of a 5-6 weeks old female Balb/c mouse. When the tumor size reached 100 mm³, animals received IV injection of MSN^{a}/siRNA-cy5/pD or Nanosac (all equivalent to siRNA 0.75 mg/kg). After 24 h, tumor, liver, heart, spleen, lung, and kidney were harvested, weighed, and frozen. The frozen tissues were homogenized in dilute HCl (pH 3) by a Qiagen TissueRuptor with disposable probes. Cy5 was retrieved from the tissue lysate by 10 sec probe sonication at 30% amplitude, followed by 72 h incubation, and quantified by Synergy Neo2 plate reader (Biotek, Chittenden County, VT, USA).

Tumor Distribution

CT26 tumor-bearing mice received a single IV injection of MSN^{a}/siRNA-cy5/pD or Nanosac (both equivalent to siRNA 0.75 mg/kg). After 24 h, the mice were injected with 100 μL of FITC-lectin (1 mg/mL in sterile saline) via tail vein. After 5 min, animals were sacrificed, and tumors were harvested, fixed in 10% neutral buffered formalin solution, infiltrated with 30% sucrose/PBS solution at 4° C., and embedded in optimal cutting temperature (OCT) compound (Fisher Scientific, Pittsburgh, PA). Cryostat sections of each tissue were obtained at a thickness of 16 μm and mounted on a glass slide. Images were taken with a Nikon A1R confocal microscope.

Statistical Analysis

All statistical analyses were performed with GraphPad Prism 8 (La Jolla, CA). All data were analyzed with one-way or two-way ANOVA test to determine the statistical difference of means among various groups, followed by the recommended multiple comparisons tests. A value of p<0.05 was considered statistically significant.

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected. It is intended that that the scope of the present methods and compositions be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### REFERENCES

1. G. J. Hannon, RNA interference. Nature 418, 244-251 (2002).
2. G. R. Devi, siRNA-based approaches in cancer therapy. Cancer Gene Therapy 13, 819-829 (2006).
3. F. L. Tan, J. Q. Yin, RNAi, a new therapeutic strategy against viral infection. Cell Research 14, 460-466 (2004).
4. M. K. Pauley, S. Cha, RNAi Therapeutics in Autoimmune Disease. Pharmaceuticals 6, (2013).
5. S. Choung, Y. J. Kim, S. Kim, H.-O. Park, Y.-C. Choi, Chemical modification of siRNAs to improve serum stability without loss of efficacy. Biochemical and Biophysical Research Communications 342, 919-927 (2006).
6. D. V. Morrissey, J. A. Lockridge, B. Polisky, Potent and persistent in vivo anti-HBV activity of chemically modified siRNAs. Nature Biotechnology 23, 1002-1007 (2005).
7. X. Zheng, C. Vladau, X. Zhang, M. Suzuki, T. E. Ichim, Z.-X. Zhang, M. Li, E. Carrier, B. Garcia, A. M. Jevnikar, W.-P. Min, A novel in vivo siRNA delivery system specifically targeting dendritic cells and silencing CD40 genes for immunomodulation. Blood 113, 2646-2654 (2009).
8. C. E. Ashley, B. Chackerian, W. Wharton, D. S. Peabody, C. J. Brinker, The targeted delivery of multicomponent cargos to cancer cells by nanoporous particle-supported lipid bilayers. Nature Materials 10, 389-397 (2011).
9. J. E. Dahlman, C. Barnes, A. Schroeder, V. Koteliansky, R. Langer, D. G. Anderson, In vivo endothelial siRNA delivery using polymeric nanoparticles with low molecular weight. Nature Nanotechnology 9, 648-655 (2014).
10. K. A. Whitehead, J. R. Dorkin, A. J. Vegas, P. H. Chang, O. Veiseh, J. Matthews, O. S. Fenton, Y. Zhang, K. T. Olejnik, V. Yesilyurt, D. Chen, S. Barros, B. Klebanov, T. Novobrantseva, R. Langer, D. G. Anderson, Degradable lipid nanoparticles with predictable in vivo siRNA delivery activity. Nature Communications 5, 4277 (2014).
11. S. M. Hoy, Patisiran: First Global Approval. Drugs 78, 1625-1631 (2018).
12. L. J. Scott, Givosiran: First Approval. Drugs 80, 335-339 (2020).
13. J. Wang, Z. Lu, M. G. Wientjes, J. L. S. Au, Delivery of siRNA Therapeutics: Barriers and Carriers. The AAPS Journal 12, 492-503 (2010).
14. K. A. Whitehead, R. Langer, D. G. Anderson, Knocking down barriers: advances in siRNA delivery. Nature Reviews Drug Discovery 8, 129-138 (2009).
15. I. Lostalé-Seijo, J. Montenegro, Synthetic materials at the forefront of gene delivery. Nature Reviews Chemistry 2, 258-277 (2018).
16. K. Singha, R. Namgung, W. J. Kim, Polymers in Small-Interfering RNA Delivery. Nucleic Acid Therapeutics 21, 133-147 (2011).
17. M. Ogris, S. Brunner, S. Schiiller, R. Kircheis, E. Wagner, PEGylated DNA/transferrin-PEI complexes: reduced interaction with blood components, extended circulation in blood and potential for systemic gene delivery. Gene Therapy 6, 595-605 (1999).
18. J. Park, J. Park, Y. Pei, J. Xu, Y. Yeo, Pharmacokinetics and biodistribution of recently-developed siRNA nanomedicines. Advanced Drug Delivery Reviews 104, 93-109 (2016).
19. J.-S. Zhang, F. Liu, L. Huang, Implications of pharmacokinetic behavior of lipoplex for its inflammatory toxicity. Advanced Drug Delivery Reviews 57, 689-698 (2005).
20. W. Yan, W. Chen, L. Huang, Mechanism of adjuvant activity of cationic liposome: Phosphorylation of a MAP kinase, ERK and induction of chemokines. Molecular Immunology 44, 3672-3681 (2007).
21. D. P. Vangasseri, Z. Cui, W. Chen, D. A. Hokey, L. D. Falo, L. Huang, Immunostimulation of dendritic cells by cationic liposomes. Molecular Membrane Biology 23, 385-395 (2006).
22. T. Tanaka, A. Legat, E. Adam, J. Steuve, J.-S. Gatot, M. Vandenbranden, L. Ulianov, C. Lonez, J.-M. Ruysschaert, E. Muraille, M. Tuynder, M. Goldman, A. Jacquet, DiC14-amidine cationic liposomes stimulate myeloid dendritic cells through Toll-like receptor 4. European Journal of Immunology 38, 1351-1357 (2008).
23. H. Hatakeyama, H. Akita, H. Harashima, The polyethyleneglycol dilemma: advantage and disadvantage of PEGylation of liposomes for systemic genes and nucleic acids delivery to tumors. Biol Pharm Bull 36, 892-899 (2013).
24. A. Lechanteur, T. Furst, B. Evrard, P. Delvenne, P. Hubert, G. Piel, PEGylation of lipoplexes: The right balance between cytotoxicity and siRNA effectiveness. Eur J Pharm Sci 93, 493-503 (2016).
25. I. M. S. Degors, C. Wang, Z. U. Rehman, I. S. Zuhorn, Carriers Break Barriers in Drug Delivery: Endocytosis and Endosomal Escape of Gene Delivery Vectors. Acc Chem Res 52, 1750-1760 (2019).
26. S. Wilhelm, A. J. Tavares, Q. Dai, S. Ohta, J. Audet, H. F. Dvorak, W. C. W. Chan, Analysis of nanoparticle delivery to tumours. Nature Reviews Materials 1, 16014 (2016).
27. A. I. Minchinton, I. F. Tannock, Drug penetration in solid tumours. Nature Reviews Cancer 6, 583-592 (2006).
28. C.-H. Heldin, K. Rubin, K. Pietras, A. Östman, High interstitial fluid pressure—an obstacle in cancer therapy. Nature Reviews Cancer 4, 806-813 (2004).
29. E. Brown, T. McKee, E. diTomaso, A. Pluen, B. Seed, Y. Boucher, R. K. Jain, Dynamic imaging of collagen and its modulation in tumors in vivo using second-harmonic generation. Nature Medicine 9, 796-800 (2003).
30. P. A. Netti, D. A. Berk, M. A. Swartz, A. J. Grodzinsky, R. K. Jain, Role of Extracellular Matrix Assembly in Interstitial Transport in Solid Tumors. Cancer Research 60, 2497 (2000).
31. Q. Sun, X. Sun, X. Ma, Z. Zhou, E. Jin, B. Zhang, Y. Shen, E. A. Van Kirk, W. J. Murdoch, J. R. Lott, T. P. Lodge, M. Radosz, Y. Zhao, Integration of Nanoassembly Functions for an Effective Delivery Cascade for Cancer Drugs. Adv Mater 26, 7615-7621 (2014).
32. Q. Zhou, S. Shao, J. Wang, C. Xu, J. Xiang, Y. Piao, Z. Zhou, Q. Yu, J. Tang, X. Liu, Z. Gan, R. Mo, Z. Gu, Y. Shen, Enzyme-activatable polymer-drug conjugate augments tumour penetration and treatment efficacy. Nature Nanotechnology 14, 799-809 (2019).
33. G. Wang, Z. Zhou, Z. Zhao, Q. Li, Y. Wu, S. Yan, Y. Shen, P. Huang, Enzyme-Triggered Transcytosis of Dendrimer-Drug Conjugate for Deep Penetration into Pancreatic Tumors. ACS Nano 14, 4890-4904 (2020).
34. Y. Hui, D. Wibowo, Y. Liu, R. Ran, H.-F. Wang, A. Seth, A. P. J. Middelberg, C.-X. Zhao, Understanding the Effects of Nanocapsular Mechanical Property on Passive and Active Tumor Targeting. ACS Nano 12, 2846-2857 (2018).
35. P. Guo, D. Liu, K. Subramanyam, B. Wang, J. Yang, J. Huang, D. T. Auguste, M. A. Moses, Nanoparticle elasticity directs tumor uptake. Nature Communications 9, 130 (2018).
36. Y. Hui, X. Yi, F. Hou, D. Wibowo, F. Zhang, D. Zhao, H. Gao, C.-X. Zhao, Role of Nanoparticle Mechanical Properties in Cancer Drug Delivery. ACS Nano 13, 7410-7424 (2019).
37. A. C. Anselmo, S. Mitragotri, Impact of particle elasticity on particle-based drug delivery systems. Advanced Drug Delivery Reviews 108, 51-67 (2017).
38. H. Deng, K. Song, J. Zhang, L. Deng, A. Dong, Z. Qin, Modulating the rigidity of nanoparticles for tumor penetration. Chem Commun 54, 3014-3017 (2018).
39. Q. Liang, N. Bie, T. Yong, K. Tang, X. Shi, Z. Wei, H. Jia, X. Zhang, H. Zhao, W. Huang, L. Gan, B. Huang, X. Yang, The softness of tumour-cell-derived microparticles regulates their drug-delivery efficiency. Nature Biomedical Engineering 3, 729-740 (2019).
40. S. Son, J. Nam, I. Zenkov, L. J. Ochyl, Y. Xu, L. Scheetz, J. Shi, O. C. Farokhzad, J. J. Moon, Sugar-Nanocapsules Imprinted with Microbial Molecular Patterns for mRNA Vaccination. Nano Letters 20, 1499-1509 (2020).
41. H. Hyun, J. Park, K. Willis, J. E. Park, L. T. Lyle, W. Lee, Y. Yeo, Surface modification of polymer nanoparticles with native albumin for enhancing drug delivery to solid tumors. Biomaterials 180, 206-224 (2018).
42. H. Meng, M. Wang, H. Liu, X. Liu, A. Situ, B. Wu, Z. Ji, C. H. Chang, A. E. Nel, Use of a Lipid-Coated Mesoporous Silica Nanoparticle Platform for Synergistic Gemcitabine and Paclitaxel Delivery to Human Pancreatic Cancer in Mice. ACS Nano 9, 3540-3557 (2015).
43. K. Grenda, J. Arnold, J. A. F. Gamelas, O. J. Cayre, M. G. Rasteiro, Flocculation of silica nanoparticles by natural, wood-based polyelectrolytes. Separation and Purification Technology 231, 115888 (2020).
44. H. Liimatainen, J. Sirviö, O. Sundman, M. Visanko, O. Hormi, J. Niinimäki, Flocculation performance of a cationic biopolymer derived from a cellulosic source in mild aqueous solution. Bioresource Technology 102, 9626-9632 (2011).
45. A.-K. Hellström, R. Bordes, Reversible flocculation of nanoparticles by a carbamate surfactant. Journal of Colloid and Interface Science 536, 722-727 (2019).
46. B. Cappella, G. Dietler, Force-distance curves by atomic force microscopy. Surface Science Reports 34, 1-104 (1999).
47. X. Liang, G. Mao, K. Y. S. Ng, Mechanical properties and stability measurement of cholesterol-containing liposome on mica by atomic force microscopy. Journal of Colloid and Interface Science 278, 53-62 (2004).
48. O. Shchepelina, M. O. Lisunova, I. Drachuk, V. V. Tsukruk, Morphology and Properties of Microcapsules with Different Core Releases. Chem Mater 24, 1245-1254 (2012).
49. Y. Xu, P. Claiden, Y. Zhu, H. Morita, N. Hanagata, Effect of amino groups of mesoporous silica nanoparticles on CpG oligodexynucleotide delivery. Science and Technology of Advanced Materials 16, 045006 (2015).
50. M. Schmid, T. K. Prinz, A. Stabler, S. Sangerlaub, Effect of Sodium Sulfite, Sodium Dodecyl Sulfate, and Urea on the Molecular Interactions and Properties of Whey Protein Isolate-Based Films. Frontiers in Chemistry 4, (2017).
51. J. C. Castle, M. Loewer, S. Boegel, J. de Graaf, C. Bender, A. D. Tadmor, V. Boisguerin, T. Bukur, P. Sorn, C. Paret, M. Diken, S. Kreiter, O. Tiireci, U. Sahin, Immunomic, genomic and transcriptomic characterization of CT26 colorectal carcinoma. BMC Genomics 15, 190 (2014).
52. M. G. Lechner, S. S. Karimi, K. Barry-Holson, T. E. Angell, K. A. Murphy, C. H. Church, J. R. Ohlfest, P. Hu, A. L. Epstein, Immunogenicity of Murine Solid Tumor Models as a Defining Feature of In Vivo Behavior and Response to Immunotherapy. Journal of Immunotherapy 36, (2013).
53. L. Ding, X. Zhu, Y. Wang, B. Shi, X. Ling, H. Chen, W. Nan, A. Barrett, Z. Guo, W. Tao, J. Wu, X. Shi, Intracellular Fate of Nanoparticles with Polydopamine Surface Engineering and a Novel Strategy for Exocytosis-Inhibiting, Lysosome Impairment-Based Cancer Therapy. Nano Letters 17, 6790-6801 (2017).
54. T. P. Szatrowski, C. F. Nathan, Production of Large Amounts of Hydrogen Peroxide by Human Tumor Cells. Cancer Research 51, 794 (1991).
55. B. Halliwell, M. V. Clement, L. H. Long, Hydrogen peroxide in the human body. FEBS Letters 486, 10-13 (2000).
56. R. Gref, A. Domb, P. Quellec, T. Blunk, R. H. Müller, J. M. Verbavatz, R. Langer, The controlled intravenous delivery of drugs using PEG-coated sterically stabilized nanospheres. Advanced Drug Delivery Reviews 16, 215-233 (1995).
57. G. Storm, S. O. Belliot, T. Daemen, D. D. Lasic, Surface modification of nanoparticles to oppose uptake by the mononuclear phagocyte system. Advanced Drug Delivery Reviews 17, 31-48 (1995).
58. D. E. Owens, N. A. Peppas, Opsonization, biodistribution, and pharmacokinetics of polymeric nanoparticles. International Journal of Pharmaceutics 307, 93-102 (2006).
59. M. A. Dobrovolskaia, P. Aggarwal, J. B. Hall, S. E. McNeil, Preclinical Studies To Understand Nanoparticle Interaction with the Immune System and Its Potential Effects on Nanoparticle Biodistribution. Molecular Pharmaceutics 5, 487-495 (2008).
60. P. Aggarwal, J. B. Hall, C. B. McLeland, M. A. Dobrovolskaia, S. E. McNeil, Nanoparticle interaction with plasma proteins as it relates to particle biodistribution, biocompatibility and therapeutic efficacy. Advanced Drug Delivery Reviews 61, 428-437 (2009).
61. Y. Jin-Wook, C. Elizabeth, M. Samir, Factors that Control the Circulation Time of Nanoparticles in Blood: Challenges, Solutions and Future Prospects. Current Pharmaceutical Design 16, 2298-2307 (2010).
62. K. Riehemann, S. W. Schneider, T. A. Luger, B. Godin, M. Ferrari, H. Fuchs, Nanomedicine—Challenge and Perspectives. Angewandte Chemie International Edition 48, 872-897 (2009).
63. W. R. Sanhai, J. H. Sakamoto, R. Canady, M. Ferrari, Seven challenges for nanomedicine. Nature Nanotechnology 3, 242-244 (2008).
64. J. Key, A. L. Palange, F. Gentile, S. Aryal, C. Stigliano, D. Di Mascolo, E. De Rosa, M. Cho, Y. Lee, J. Singh, P. Decuzzi, Soft Discoidal Polymeric Nanoconstructs Resist Macrophage Uptake and Enhance Vascular Targeting in Tumors. ACS Nano 9, 11628-11641 (2015).
65. L. Zhang, Z. Cao, Y. Li, J.-R. Ella-Menye, T. Bai, S. Jiang, Softer Zwitterionic Nanogels for Longer Circulation and Lower Splenic Accumulation. ACS Nano 6, 6681-6686 (2012).
66. N. F. Bouxsein, C. S. McAllister, K. K. Ewert, C. E. Samuel, C. R. Safinya, Structure and Gene Silencing Activities of Monovalent and Pentavalent Cationic Lipid Vectors Complexed with siRNA. Biochemistry 46, 4785-4792 (2007).
67. S. M. Moghimi, A. C. Hunter, T. L. Andresen, Factors controlling nanoparticle pharmacokinetics: an integrated analysis and perspective. Annu Rev Pharmacol Toxicol 52, 481-503 (2012).
68. A. C. Anselmo, M. Zhang, S. Kumar, D. R. Vogus, S. Menegatti, M. E. Helgeson, S. Mitragotri, Elasticity of Nanoparticles Influences Their Blood Circulation, Phagocytosis, Endocytosis, and Targeting. ACS Nano 9, 3169-3177 (2015).
69. Y. Matsumoto, J. W. Nichols, K. Toh, T. Nomoto, H. Cabral, Y. Miura, R. J. Christie, N. Yamada, T. Ogura, M. R. Kano, Y. Matsumura, N. Nishiyama, T. Yamasoba, Y. H. Bae, K. Kataoka, Vascular bursts enhance permeability of tumour blood vessels and improve nanoparticle delivery. Nature Nanotechnology 11, 533-538 (2016).
70. Y. Hui, X. Yi, D. Wibowo, G. Yang, A. P. J. Middelberg, H. Gao, C.-X. Zhao, Nanoparticle elasticity regulates phagocytosis and cancer cell uptake. Science Advances 6, eaaz4316 (2020).
71. P. Vader, E. A. Mol, G. Pasterkamp, R. M. Schiffelers, Extracellular vesicles for drug delivery. Advanced Drug Delivery Reviews 106, 148-156 (2016).
72. G. Stehle, H. Sinn, A. Wunder, H. H. Schrenk, J. C. M. Stewart, G. Hartung, W. Maier-Borst, D. L. Heene, Plasma protein (albumin) catabolism by the tumor itself-implications for tumor metabolism and the genesis of cachexia. Critical Reviews in Oncology/Hematology 26, 77-100 (1997).
73. C. Commisso, S. M. Davidson, R. G. Soydaner-Azeloglu, S. J. Parker, J. J. Kamphorst, S. Hackett, E. Grabocka, M. Nofal, J. A. Drebin, C. B. Thompson, J. D. Rabinowitz, C. M. Metallo, M. G. Vander Heiden, D. Bar-Sagi, Macropinocytosis of protein is an amino acid supply route in Ras-transformed cells. Nature 497, 633-637 (2013).
74. S. M. Vogel, R. D. Minshall, M. Pilipović, C. Tiruppathi, A. B. Malik, Albumin uptake and transcytosis in endothelial cells in vivo induced by albumin-binding protein. American Journal of Physiology-Lung Cellular and Molecular Physiology 281, L1512-L1522 (2001).
75. Y. Komarova, A. B. Malik, Regulation of Endothelial Permeability via Paracellular and Transcellular Transport Pathways. Annual Review of Physiology 72, 463-493 (2010).
76. C. Tiruppathi, W. Song, M. Bergenfeldt, P. Sass, A. B. Malik, Gp60 Activation Mediates Albumin Transcytosis in Endothelial Cells by Tyrosine Kinase-dependent Pathway. Journal of Biological Chemistry 272, 25968-25975 (1997).
77. J. E. Schnitzer, P. Oh, Albondin-mediated capillary permeability to albumin. Differential role of receptors in endothelial transcytosis and endocytosis of native and modified albumins. Journal of Biological Chemistry 269, 6072-6082 (1994).
78. S. Sindhwani, A. M. Syed, J. Ngai, B. R. Kingston, L. Maiorino, J. Rothschild, P. MacMillan, Y. Zhang, N. U. Rajesh, T. Hoang, J. L. Y. Wu, S. Wilhelm, A. Zilman, S. Gadde, A. Sulaiman, B. Ouyang, Z. Lin, L. Wang, M. Egeblad, W. C. W. Chan, The entry of nanoparticles into solid tumours. Nature Materials 19, 566-575 (2020).
79. K.-i. Ogawara, K. Furumoto, S. Nagayama, K. Minato, K. Higaki, T. Kai, T. Kimura, Pre-coating with serum albumin reduces receptor-mediated hepatic disposition of polystyrene nanosphere: implications for rational design of nanoparticles. Journal of Controlled Release 100, 451-455 (2004).
80. K. Furumoto, J.-I. Yokoe, K.-i. Ogawara, S. Amano, M. Takaguchi, K. Higaki, T. Kai, T. Kimura, Effect of coupling of albumin onto surface of PEG liposome on its in vivo disposition. International Journal of Pharmaceutics 329, 110-116 (2007).
81. H. Beukers, F. A. Deierkauf, C. P. Blom, M. Deierkauf, J. C. Riemersma, Effects of albumin on the phagocytosis of polysterene spherules by rabbit polymorphonuclear leucocytes. Journal of Cellular Physiology 97, 29-36 (1978).
82. Q. Peng, S. Zhang, Q. Yang, T. Zhang, X.-Q. Wei, L. Jiang, C.-L. Zhang, Q.-M. Chen, Z.-R. Zhang, Y.-F. Lin, Preformed albumin corona, a protective coating for nanoparticles based drug delivery system. Biomaterials 34, 8521-8530 (2013).
83. W. Schubert, P. G. Frank, B. Razani, D. S. Park, C.-W. Chow, M. P. Lisanti, Caveolae-deficient Endothelial Cells Show Defects in the Uptake and Transport of Albumin in Vivo. Journal of Biological Chemistry 276, 48619-48622 (2001).
84. M. Chatterjee, E. Ben-Josef, R. Robb, M. Vedaie, S. Seum, K. Thirumoorthy, K. Palanichamy, M. Harbrecht, A. Chakravarti, T. M. Williams, Caveolae-Mediated Endocytosis Is Critical for Albumin Cellular Uptake and Response to Albumin-Bound Chemotherapy. Cancer Research 77, 5925 (2017).
85. N. Desai, V. Trieu, B. Damascelli, P. Soon-Shiong, SPARC Expression Correlates with Tumor Response to Albumin-Bound Paclitaxel in Head and Neck Cancer Patients. Translational Oncology 2, 59-64 (2009).
86. A. L. Kiss, E. Botos, Endocytosis via caveolae: alternative pathway with distinct cellular compartments to avoid lysosomal degradation? Journal of Cellular and Molecular Medicine 13, 1228-1237 (2009).
87. A. Hayer, M. Stoeber, D. Ritz, S. Engel, H. H. Meyer, A. Helenius, Caveolin-1 is ubiquitinated and targeted to intralumenal vesicles in endolysosomes for degradation. Journal of Cell Biology 191, 615-629 (2010).
88. L. Pelkmans, J. Kartenbeck, A. Helenius, Caveolar endocytosis of simian virus 40 reveals a new two-step vesicular-transport pathway to the ER. Nature Cell Biology 3, 473-483 (2001).
89. C. Park, S. Marqusee, Pulse proteolysis: A simple method for quantitative determination of protein stability and ligand binding. Nature Methods 2, 207-212 (2005).
90. J. Cox, M. Mann, MaxQuant enables high peptide identification rates, individualized p.p.b.-range mass accuracies and proteome-wide protein quantification. Nature Biotechnology 26, 1367-1372 (2008).
91. A. Au-Belkacemi, M. W. Au-Laschke, M. D. Au-Menger, V. Au-Flockerzi, Scratch Migration Assay and Dorsal Skinfold Chamber for In Vitro and In Vivo Analysis of Wound Healing. JoVE, e59608 (2019).
92. A. L. B. Au-Seynhaeve, T. L. M. Au-ten Hagen, Intravital Microscopy of Tumor-associated Vasculature Using Advanced Dorsal Skinfold Window Chambers on Transgenic Fluorescent Mice. JoVE, e55115 (2018).
93. G. M. Palmer, A. N. Fontanella, S. Shan, G. Hanna, G. Zhang, C. L. Fraser, M. W. Dewhirst, In vivo optical molecular imaging and analysis in mice using dorsal window chamber models applied to hypoxia, vasculature and fluorescent reporters. Nature Protocols 6, 1355-1366 (2011).
94. M. M. Tomayko, C. P. Reynolds, Determination of subcutaneous tumor size in athymic (nude) mice. Cancer Chemother Pharmacol 24, 148-154 (1989).

What is claimed is:

1. A method of manufacturing soft, non-cationic nanocapsules (Nanosac) for in vivo delivery of a therapeutic compound (TC) comprising:
preparing the therapeutic compound (TC) to be delivered;
preparing a mesoporous silica nanoparticle (MSN);
coating the MSN with the TC to afford TC-MSN;
coating the TC-MSN with polydopamine (pD) to afford pD-TC-MSN; and
dispersing the pD-TC-MSN in a buffered oxide etch solution to remove MSN and affording the Nanosac with the therapeutic compound (TC).

2. The method of claim 1, wherein the TC is a small molecule drug or a biologic.

3. The method of claim 2, wherein the small molecule drug comprises paclitaxel, sorafenib, itraconazole, docetaxel, doxorubicin, bortezomib, carfilzomib, camptothecin, cisplatin, oxaliplatin, cytarabine, vincristine, irinotecan, amphotericin, and gemcitabine.

4. The method of claim 2, wherein the TC is a therapeutic molecule selected from the group consisting of antibody therapeutics, peptide therapeutics, protein therapeutics, therapeutic RNAs, therapeutic DNAs, and therapeutic enzymes.

5. The method of claim 2, wherein the biologic is a small interfering RNA (siRNA).

6. The method of claim 2, wherein the biologic is a cytokine selected from the group consisting of interleukin-2 (IL-2), interferon-α (IFN-α), IL-15, IL-21, and IL-12.

7. The method of claim 2, wherein the biologic is an antibody selected from the group consisting of rituximab, trastuzumab, gemtuzumab ozogamicin, alemtuzumab, tositumomab, cetuximab, ibritumomab tiuxetan, bevacizumab, panitumumab, catumaxomab, ofatumumab, ipilimumab, and brentuximab vedoitin.

8. The method of claim 4, wherein the therapeutic RNA is a messenger RNA (mRNA).

9. The method of claim 4, wherein the therapeutic RNAs are non-coding RNAs selected from the group consisting of small-interfering RNAs (siRNAs), microRNAs (miRNAs), piwi-interacting RNAs (piRNAs), enhancer RNAs (eRNAs), long non-coding RNAs (lncRNAs), and circular RNA (circRNAs).

10. The method of claim 1, wherein the Nanosac is useful for systemic delivery of a therapeutic molecule selected from the group consisting of small molecular drugs, antibody therapeutics, peptide therapeutics, protein therapeutics, therapeutic enzymes, and therapeutic nucleic acids (DNAs, RNAs).

11. The method of claim 1, wherein the Nanosac is useful as a cancer treatment.

12. The process of claim 1, wherein the Nanosac is useful as a treatment for diseases caused by viral and bacterial infections.

13. (canceled)

14. A composition of soft, non-cationic nanocapsules (Nanosac) useful for in vivo delivery of a therapeutic compound (TC) manufactured according to the steps of
preparing the TC to be delivered;
preparing an amine modified mesoporous silica nanoparticle (MSN);
coating the MSN with the TC to afford TC-MSN;
coating the TC-MSN with polydopamine (pD) to afford pD-TC-MSN; and
dispersing the pD-TC-MSN in a buffered oxide etch solution to remove MSN and affording the Nanosac with the TC.

15. The composition of claim 14, wherein the TC is a small molecule drug or a biologic.

16. The composition of claim 15, wherein the small molecule drug comprises paclitaxel, sorafenib, itraconazole, docetaxel, doxorubicin, bortezomib, carfilzomib, camptothecin, cisplatin, oxaliplatin, cytarabine, vincristine, irinotecan, amphotericin, and gemcitabine.

17. The composition of claim 15, wherein the TC is a therapeutic molecule selected from the group consisting of antibody therapeutics, peptide therapeutics, protein therapeutics, therapeutic RNAs, therapeutic DNAs, therapeutic interfering RNAs, and therapeutic enzymes.

18. The composition of claim 15, wherein the biologic is an interfering RNA.

19. The composition of claim 15, wherein the biologic is a small interfering RNA (siRNA).

20. The composition of claim 14, wherein the Nanosac is useful for systemic delivery of a therapeutic treatment selected from the group consisting of small molecular drugs, antibody therapeutics, peptide therapeutics, protein therapeutics, therapeutic RNAs, therapeutic DNAs, therapeutic interfering RNAs, and therapeutic enzymes.

21. The composition of claim 14, wherein the composition is useful as a cancer treatment.

22. (canceled)

23. The composition of claim 14, wherein the composition is a pharmaceutical composition useful as a cancer treatment.

24. The composition of claim 14, wherein the Nanosac is useful as a treatment for diseases caused by viral and bacterial infections.

25. A method of intratumoral delivery of a therapeutic compound (TC) to a patient comprising preparing a dosage form of a composition of soft, non-cationic nanocapsules (Nanosac), wherein the Nanosac is manufactured according to a process of:
preparing the TC to be delivered;
preparing an amine modified mesoporous silica nanoparticle (MSN);
coating the MSN with an amount of the TC to afford TC-MSN;
coating the TC-MSN with polydopamine (pD) to afford pD-TC-MSN; and
dispersing the pD-TC-MSN in a buffered oxide etch solution to remove MSN and affording the Nanosac with the TC; and
systemically delivering the Nanosac composition to a tumor of the patient in a dose sufficient to achieve a therapeutic response.

26. The method of claim 25, wherein the TC is a small molecule drug or a biologic.

27. The method of claim 26, wherein the small molecule drug comprises paclitaxel, sorafenib, itraconazole, docetaxel, doxorubicin, bortezomib, carfilzomib, camptothecin, cisplatin, oxaliplatin, cytarabine, vincristine, irinotecan, amphotericin, and gemcitabine.

28. The method of claim 26, wherein the TC is a therapeutic molecule selected from the group consisting of antibody therapeutics, peptide therapeutics, protein therapeutics, therapeutic RNAs, therapeutic DNAs, therapeutic interfering RNAs, and therapeutic enzymes.

29. The method of claim 26, wherein the biologic is a small interfering RNA (siRNA).

30. (canceled)

31. (canceled)

32. (canceled)

33. The method of claim 1, wherein the MSN is an amine-modified mesoporous silica nanoparticle.


SEQUENCE LISTING (4 sequences)

SEQ ID NO: 1 - DNA, 21 bp, Artificial sequence
  PD-L1 siRNA, sense
cccacauaaaaaacaguugtt

SEQ ID NO: 2 - DNA, 21 bp, Artificial sequence
  PD-L1 siRNA, antisense
caacuguuuuuuaugugggtt

SEQ ID NO: 3 - DNA, 21 bp, Artificial sequence
  negative control siRNA, sense
  n (20)..(20): deoxythymidine
  misc_feature (20)..(21): n is a, c, g, t or u
  n (21)..(21): deoxythymidine
ugaaguugcacuugaagucnn

SEQ ID NO: 4 - DNA, 21 bp, Artificial sequence
  Negative control siRNA, antisense
  n (20)..(20): deoxythymidine
  misc_feature (20)..(21): n is a, c, g, t or u
  n (21)..(21): deoxythymidine
gacuucaagugcaacuucann